(19) United States
(12) Patent Application Publication
(10) Pub. No.: US 2025/0382628 A1
(43) Pub. Date: Dec. 18, 2025

(54) RUST DISEASE RESISTANCE GENES AND USE THEREOF

(71) Applicant: Ramot at Tel-Aviv University Ltd., Tel Aviv (IL)

(72) Inventors: Amir SHARON, Tel Aviv (IL); Raz AVNI, Tel Aviv (IL); Anna MINZ-DUB, Tel Aviv (IL); Eitan MILLET, Tel Aviv (IL); Davinder SHARMA, Tel Aviv (IL); Rakesh KUMAR, Tel Aviv (IL)

(21) Appl. No.: 18/876,444

(22) PCT Filed: Jun. 7, 2023
(86) PCT No.: PCT/IL2023/050584

§ 371 Date: Dec. 18, 2024

Related U.S. Application Data
(60) Provisional application No. 63/354,703, filed on Jun. 23, 2022.

(51) Int. Cl. C12N 15/82; C07K 14/415; C12Q 1/6895

(52) U.S. Cl. CPC C12N 15/8282; C07K 14/415; C12N 15/8209; C12Q 1/6895; C12Q 2600/13

(57) ABSTRACT

The present invention relates to polynucleotides which, when expressed in a Triticeae plant cell, particularly wheat, barley or rye plant cells confer or enhance resistance or tolerance towards leaf rust disease and/or stripe rust disease, and to Triticeae plants comprising the polynucleotides that are tolerant or resistant to the rust diseases, as well as to methods of producing same and of identifying resistant plants.

### FIELD OF THE INVENTION

The present invention relates to polynucleotides encoding proteins which, when expressed in a Triticeae plant cell, particularly wheat and barley plant cells, enhance tolerance and/or confer resistance towards the rust diseases leaf rust and stripe rust, and to Triticeae plants comprising the polynucleotides that are tolerant or resistant to the rust diseases, as well as to methods of producing same and of identifying resistant plants.

### BACKGROUND OF THE INVENTION

Bread wheat (Triticum aestivum L.) is one of the most important food crops in the world, providing almost one fifth of the calories consumed by humans. However, wheat is susceptible to many diseases that cause huge global yield losses. Leaf rust (Lr), caused by the fungus Puccinia triticina Erikss. (Pt) and stripe (yellow) rust, caused by Puccinia striiformis Westend. f. sp. tritici Erikss., (Pst), are two of the most widespread and devastating wheat diseases, each causing tremendous yield losses annually. The severity and spreading of stripe rust have been intensified in the past years, and was accompanied by appearance of new and highly virulent races of the pathogen. Outbreaks and local epidemics of Stripe rust took place all over the globe with severe outbreaks in Australia,

China, Pakistan, Central and West Asia, the Middle East (Syria and Turkey), India and U.S.A., indicating virulence changes of the pathogen (Wellings C R et al., 2012, In: Disease resistance in wheat. Ed. Sharma I. CABI, Wallingford, 63-83). It was shown that the new stripe rust strains became adapted to higher inoculation temperatures that might have facilitated spreading of the disease to new areas (Milus E A et al., 2006, Plant Disease, 90 (7): 847-852).

Breeding for rust resistance is the most economical and environmentally safe way to control rust diseases. Genetic diversity for disease resistance can be increased by sourcing new genes from wheat wild relatives. While the primary wheat gene pool (species with identical genome(s) to wheat) has been extensively exploited in breeding programs, the secondary gene pool remains a rich source of novel disease resistance genes (Vikas et al., 2014, Genetic resources and crop evolution, 61(4):861-874). One of the most attractive sources within the secondary gene pool are species of the Sitopsis section of Aegilops that contain many useful traits, in particular for disease resistance and abiotic stress tolerance. The Sitopsis species have diploid homoeologous genomes (S or S*), which complicates gene transfer to wheat and therefore only part of the potential rust resistance genes has been exploited (Feuillet et al., 2007 Israel Journal of Plant Sciences, 55(3-4):307-313; Friebe et al., 1996, Euphytica, 91(1), 59-87; Millet E. 2007, Israel Journal of Plant Sciences, 55(3-4), 277-287). Rust resistance genes that have been transferred to wheat from these species include the stem rust resistance (Sr) genes Sr32, Sr39, Sr47 and Sr51; the leaf rust resistance (Lr) genes Lr28, Lr35, Lr36, Lr47, Lr51 and Lr56; the stripe rust resistance (Yr) gene Yr38, and the powdery mildew resistance (Pm) genes Pm12, Pm32 and Pm 13 (Klindworth et al. 2012, G3: Gene Genomes Genetics, 2(6), 665-673; Liu et al. 2011, Theoretical and Applied Genetics, 122(8), 1537-1545; Millet ibid; Schneider et al. 2008, Euphytica 163(1): 1-19).

Elongated goatgrass (Aegilops longissima Schweinf. & Muschl.) (AEL) is one of the five species in section Sitopsis. It has a wide ecological preference, which includes the coastal plains of Egypt, Israel and Lebanon, and sandstone and limestone soils of Jordan (van Slageren M W., 1994, Wild wheats: A monograph of Aegilops L. and Amblyopyrum (Jaub. & Spach) Eig (Poaceae). Wageningen Agric. Univ. Papers, Wageningen, Netherlands). Recent work by Huang S et al (2018, Plant disease, 102(6):1124-1135) on a diverse collection of Ae. longissima lines revealed that many accessions are highly resistant to inoculation with leaf, stripe or stem rust pathogens. This finding was in line with previous reports of resistance found in Ae. longissima against stem rust (Anikster et al 2005, Plant disease, 89(3):303-308; Scott et al., 2014, Plant disease, 98(10):1309-1320); leaf rust (Anikster et al., 2005 ibid); stripe rust (Anikster et al., 2005, ibid); powdery mildew (Ceoloni et al., 1992, Hereditas, 116:239-245); Septoria blotch (Ecker et al., 1990; Plant Breeding, 104 (3): 224-230; McKendry and Henke 1994, Crop Science, 34(4):1080-1084); and eye spot (Sheng and Murray 2013, Plant disease, 97(3):346-353; Sheng et al 2012, Theoretical and Applied Genetics, 125(2):355-366; Sheng et al 2014, Theoretical and applied genetics, 127(10):2085-2093). Moreover, genetic analysis of the closely related species Aegilops sharonensis demonstrated monogenic inheritance of rust resistance genes (Olivera et al., 2008, Phytopathology 98:353-358).

The dynamic nature of the pathogenic fungi causing rust diseases requires active efforts for developing new control means and there is an ongoing research in this direction. For Example, U.S. Pat. No. 10,760,093 and International (PCT) Application Publication No. WO 2021/001832 to inventors of the present invention disclose chromosome segment and polynucleotides derived therefrom of Ae. sharonensis, that confers, enhances, or otherwise facilitates resistance of wheat plants to leaf rust and/or stripe rust disease.

U.S. Patent Application Publication No. 2018/0320195 discloses compositions and methods for enhancing the resistance of wheat and barley plants to wheat stripe rust caused by Puccinia striiformis f. sp. tritici. The compositions comprise nucleic acid molecules encoding resistance (R) gene products and variants thereof and plants, seeds, and plant cells comprising such nucleic acid molecules. The methods for enhancing the resistance of wheat and barley plants to wheat stripe rust comprise introducing a nucleic acid molecule encoding an R gene product into a wheat or barley plant cell. Methods for using the wheat and barley plants in agriculture to limit wheat stripe rust are also provided.

U.S. Patent Application Publication No. 2020/0362367 and U.S. Pat. No. 11,236,356 disclose compositions and methods for enhancing the resistance of wheat plants to wheat stem rust caused by Puccinia graminis f. sp. tritici.

There is a great need for and would be highly advantageous to have new genes which enhances the tolerance or confer resistance to plants, especially wheat plants, to rust diseases.

### SUMMARY OF THE INVENTION

The present invention provides isolated polynucleotide molecules encoding products that confer, enhance, or otherwise facilitate the tolerance and/or resistance of Triticeae plants and cultivars comprising these polynucleotides in at least part of the plant cells to rust diseases, particularly to leaf rust and stripe rust diseases. The present invention further provides Triticeae plants that are tolerant and/or resistant to virulent forms of Puccinia fungi inducing leaf rust and/or stripe rust diseases, including resistant elite Triticeae cultivars. The conferred tolerance/resistance is manifested throughout the plant growth period—from seedlings to mature plants. In certain aspects, the present invention further provides methods of producing the rust-disease tolerant/resistant Triticeae plant and methods of selecting same. In certain embodiments, the Triticeae plant is wheat (family Triticum), barely plant (family Horedum) or rye (Secale cereale). In certain currently exemplary embodiments, the Triticeae plant is wheat.

The present invention is based in part on the discovery of a gene within the genome of Aegilops longissima which enhances the tolerance of wheat plants to several races of each of Puccinia triticina and Puccinia striiformis which cause wheat leaf rust disease and stripe rust disease, respectively, designated herein Lr/Yr548. Unexpectedly, the gene, belonging to the nucleotide-binding site leucine-rich repeat (NLR) gene family, known to confer resistance against races of a single pathogen, enhances the tolerance to both leaf rust and stripe rust diseases.

According to certain aspects, the present invention provides a Triticeae plant comprising at least one cell comprising a heterologous polynucleotide encoding a polypeptide having at least 80% identity to the amino acid sequence set forth in SEQ ID NO: 1, wherein the heterologous polynucleotide is capable of conferring or enhancing tolerance and/or resistance of the plant to at least one rust disease.

According to some embodiments, the encoded polypeptide comprises an amino acid sequence having at least 85%, at least 90% or at least 95% identity to the amino acids sequence set forth in SEQ ID NO:1.

According to certain exemplary embodiments, the encoded polypeptide comprises the amino acid sequence set forth in SEQ ID NO:1. According to further certain exemplary embodiments, the encoded polypeptide consists of the amino acid sequence set forth in SEQ ID NO:1.

According to certain embodiments, the heterologous polynucleotide comprises a nucleic acid sequence having at least 70% identity to the nucleic acid sequence set forth in SEQ ID NO:6 over the entire length of the polynucleotide.

According to certain embodiments, the heterologous polynucleotide comprises a nucleic acid sequence having at least 70% identity to the nucleic acid sequence set forth in SEQ ID NO:6 over introns comprised within the sequence having SEQ ID NO:7, SEQ

ID NO: 8 and SEQ ID NO:9; and at least 88% identity over exons comprised within said sequence having SEQ ID NO:10, SEQ ID NO:11, SEQ ID NO: 12 and SEQ ID NO:13.

According to certain embodiments, the intron having SEQ ID NO:7 is located at positions 295-5,327 of SEQ ID NO:6. According to certain embodiments, the intron having SEQ ID NO:8 is located at positions 6,484-13,006 of SEQ ID NO:6. According to certain embodiments, the intron having SEQ ID NO:9 is located at positions 16,008-16,124 of SEQ ID NO:6.

According to certain embodiments, the exon having SEQ ID NO: 10 is located at positions 1-294 of SEQ ID NO:6. According to certain embodiments, the exon having SEQ ID NO: 11 is located at positions 5,328-6,483 of SEQ ID NO:6. According to certain embodiments, the exon having SEQ ID NO: 12 is located at positions 13,007-16,007 of SEQ ID NO:6. According to certain embodiments, the exon having SEQ ID NO:13 is located at positions 16,125-16,207 of SEQ ID NO:6.

According to some embodiments, the heterologous polynucleotide comprises a nucleic acid sequence having at least 89%, at least 90%, at least 91%, at least 92%, at least 93%, at least 94%, at least 95%, at least 96%, at least 97%, at least 98%, or at least 99% identity to the exons comprised within the nucleic acid sequence set forth in SEQ ID NO: 6. Each possibility represents a separate embodiment of the present invention.

According to certain embodiments, the heterologous polynucleotide comprises the nucleic acid sequence set forth in SEQ ID NO:6. According to certain embodiments, the heterologous polynucleotide consists of the nucleic acid sequence set forth in SEQ ID NO: 6.

According to some embodiments, the heterologous polynucleotide comprises a nucleic acid sequence having at least 88% identity to the nucleic acid sequence set forth in SEQ ID NO:3 over its entire length.

According to some embodiments, the heterologous polynucleotide comprises a nucleic acid sequence having at least 89%, at least 90%, at least 91%, at least 92%, at least 93%, at least 94%, at least 95%, at least 96%, at least 97%, at least 98%, or at least 99% identity to the nucleic acid sequence set forth in SEQ ID NO:3 over its entire length. Each possibility represents a separate embodiment of the present invention.

According to certain embodiments, the heterologous polynucleotide comprises the nucleic acid sequence set forth in SEQ ID NO:3. According to certain embodiments, the heterologous polynucleotide consists of the nucleic acid sequence set forth in SEQ ID NO: 3.

Introducing the heterologous polynucleotide capable of enhancing or conferring tolerance and/or resistance to a Triticeae plant towards at least one rust disease may be achieved by various means, all of which are explicitly encompassed within the scope of the present invention.

According to certain embodiments, the heterologous polynucleotide is introduced into the Triticeae plant by means of transformation of said heterologous polynucleotide into at least one cell of the plant. According to these embodiments, the heterologous polynucleotide is operably linked to at least one regulatory element capable of controlling expression of said polynucleotide in a cell of the plant, thereby forming a DNA construct or an expression vector. The at least one regulatory element can be endogenous or heterologous to the plant cell. According to certain embodiments, the at least one regulatory element is a promoter. According to certain embodiments, the promoter is derived from Aegilops longissima. According to certain exemplary embodiments, the promoter is the natural promoter deriving the expression of the tolerance/resistance conferring gene in Ae. longissima. According to these embodiments, the promoter comprises a nucleic acid sequence having at least 90% identity to the nucleic acid sequence set forth in SEQ ID NO:4. According to certain currently exemplary embodiments, the promoter comprises the nucleic acid sequence set forth in SEQ ID NO: 4. According to certain currently exemplary embodiments, the promoter consists of the nucleic acid sequence set forth in SEQ ID NO:4.

According to certain embodiments, the at least one regulatory element is a transcription terminator. According to certain embodiments, the terminator is derived from Aegilops longissima. According to certain exemplary embodiments, the terminator is the natural terminator of the tolerance/resistance conferring gene in Ae. longissima. According to these embodiments, the terminator comprises a nucleic acid sequence having at least 90% identity to the nucleic acid sequence set forth in SEQ ID NO:5. According to certain currently exemplary embodiments, the terminator comprises the nucleic acid sequence set forth in SEQ ID NO:5. According to further certain currently exemplary embodiments, the terminator consists of the nucleic acid sequence set forth in SEQ ID NO:5.

According to these embodiments, the Triticeae plant is a transgenic plant comprising at least one cell transformed with a heterologous polynucleotide encoding a polypeptide having at least 80% identity to the amino acid sequence set forth in SEQ ID NO: 1, a construct or an expression vector comprising same.

The heterologous polynucleotide is as described hereinabove.

According to certain embodiments, the heterologous polynucleotide is introduced into the at least one cell of the Triticeae plant by means of genome editing using artificially engineered nucleases as is known in the art. According to certain embodiments, the artificially engineered nucleases are selected from the group consisting of meganucleases, Zinc finger nucleases (ZFNs), transcription-activator like effector nucleases (TALENs), and Clustered Regularly Interspaced Short Palindromic Repeats (CRISPR)/Cas, CRISPR/Cas homologous and CRISPR/Cas modified systems. Each possibility represents a separate embodiment of the present invention. Since most genome-editing techniques can leave behind minimal traces of DNA alterations evident in a small number of nucleotides as compared to transgenic plants, crop plants created through gene editing for expression of the heterologous polynucleotides of the invention could avoid the stringent regulation procedures commonly associated with genetically modified (GM) crop development, and are typically defined as non-transgenic crop plants, particularly non-transgenic crop wheat plants.

According to certain embodiments, the Triticeae plant is homozygous for the heterologous polynucleotide capable of enhancing or conferring tolerance and/or resistance to the at least one rust disease.

According to certain embodiments, the Triticeae plant is heterozygous for the heterologous polynucleotide capable of enhancing or conferring tolerance and/or resistance to the at least one rust disease.

According to certain embodiments, the Triticeae plant comprising within at least one of its cells a heterologous polynucleotide of the invention has enhanced tolerance and/or resistance to at least one rust disease compared to a corresponding Triticeae plant devoid of the heterologous polynucleotide. According to certain exemplary embodiments, the Triticeae plant comprising within at least one of its cells a heterologous polynucleotide of the invention has enhanced tolerance and/or resistance to leaf rust disease and stripe rust disease compared to a corresponding Triticeae plant devoid of the heterologous polynucleotide.

According to certain embodiments, the corresponding plant devoid of the heterologous polynucleotide is of the same species. According to certain embodiments, the corresponding plant devoid of the heterologous polynucleotide has the same genetic background.

According to certain embodiments, the Triticeae plant is selected from the group consisting of wheat (Triticum), barely (Hordeum), and rye (Secale cereale). Each possibility represents a separate embodiment of the present invention.

According to certain exemplary embodiments, the Triticeae plant of the present invention is a wheat cultivar suitable for commercial agricultural growth, but it is not restricted to a specific plant species, strain, or variety. According to certain exemplary embodiments, the wheat cultivar comprising the heterologous polynucleotide is of a species selected from the group consisting of Triticum turgidum and Triticum aestivum.

Each possibility represents a separate embodiment of the present invention. According to certain embodiments, the wheat plant is an elite agricultural cultivar.

According to yet further exemplary embodiment, the Triticeae plant of the present invention is a barley cultivar suitable for commercial agricultural growth, but it is not restricted to a specific plant species, strain, or variety. According to certain exemplary embodiments, the barely cultivar is of the species Hordeum vulgare.

According to certain embodiments, the at least one rust disease is selected from the group consisting of leaf rust disease, stripe rust disease and a combination thereof. Each possibility represents a separate embodiment of the present invention.

According to certain exemplary embodiments, the rust disease is leaf rust disease. According to certain embodiments, the leaf rust disease is caused by the fungus Puccinia triticina (Pt). According to certain currently exemplary embodiments, the Puccinia triticina is of a race selected from the group consisting of race #12460 (MCDTB), race #12337 (MFPPB), race #526-24 (MFBKG) and any combination thereof. Each possibility represents a separate embodiment of the present invention.

According to certain embodiments, the rust disease is stripe rust disease. According to certain embodiments, stripe rust disease is caused by the fungus Puccinia striiformis (Pst). According to certain currently exemplary embodiments, the Puccinia striiformis is of the race #5006.

According to certain exemplary embodiments, the Triticeae plant of the present invention have enhanced tolerance/resistance to leaf rust disease and stripe rust disease.

The Triticeae plants and cultivars of the present invention are fertile, or male sterile that will produce seeds upon pollination. Seeds and any other plant part that can be used for propagation, including isolated cells and tissue cultures are also encompassed within the scope of the present invention. It is to be understood that a plant produced from said seeds or other propagating material comprises the heterologous polynucleotide that is capable to confers or enhances tolerance and/or resistance to at least one rust disease as described herein is encompassed within the teachings of the present invention.

According to additional aspects, the present invention provides a method for producing a Triticeae plant having enhanced resistance to at least one rust diseases, the method comprises introducing into at least one cell of a Triticeae plant susceptible to the at least one rust diseases a heterologous polynucleotide encoding a polypeptide having at least 80% identity to the amino acid sequence set forth in SEQ ID NO: 1, a DNA construct or a vector comprising same, thereby producing a Triticeae plant having enhanced tolerance and/or resistance to said at least one rust disease compared to a corresponding control plant.

According to certain exemplary embodiments, the produced Triticeae plant has enhanced tolerance and/or resistance to leaf rust disease and stripe rust disease.

According to certain currently exemplary embodiments, the heterologous polynucleotide encodes a polypeptide having the amino acid sequence set forth in SEQ ID NO: 1.

The polynucleotides and the Triticeae plants are as described hereinabove.

According to certain embodiments, the Triticeae plant is selected from wheat and barley. Each possibility represents a separate embodiment of the present invention.

According to certain embodiments, the wheat plant and/or the barley is an agricultural cultivar as described hereinabove.

According to certain embodiments, the control plant is a Triticeae plant or cultivar susceptible to the at least one rust disease. According to some embodiments, the control plant is lacking the heterologous polynucleotide. According to certain embodiments, the control plant is lacking the heterologous polynucleotide while having the same genetic background.

Any method as is known to a person skilled in the art can be used to introduce the heterologous polynucleotide of the present invention into a susceptible Triticeae plant.

According to certain embodiments, the heterologous polynucleotide is comprised within a DNA construct and/or an expression vector. According to certain embodiments, the heterologous polynucleotide is introduced by transforming said isolated polynucleotide, DNA construct or expression vector into at least one cell of the susceptible Triticeae plant. According to certain embodiments, the isolated polynucleotide is introduced by subjecting at least one cell of the susceptible Triticeae plant to genome editing using artificially engineered nucleases.

According to certain additional aspects, the present invention provides a method for selecting a Triticeae plant having an enhanced tolerance and/or resistance to at least one rust diseases, comprising the steps of:
a. providing a plurality of Triticeae plants each comprising at least one cell comprising a heterologous polynucleotide encoding a polypeptide having at least 80% identity to the amino acid sequence set forth in SEQ ID NO:1, wherein the polynucleotide is capable of conferring or enhancing tolerance and/or resistance to the Triticeae plants towards at least one rust disease,; and
b. selecting plants showing an enhanced resistance to said at least one rust disease compared to a control Triticeae plant or to a pre-determined resistance score value;
thereby selecting a Triticeae plant having enhanced resistance to said at least one rust disease.

According to certain embodiments, the control plant is a Triticeae plant susceptible to the at least one rust disease. According to some embodiments, the susceptible control Triticeae plant is lacking the heterologous polynucleotide while having the same genetic background.

The heterologous polynucleotide and the Triticeae plants are as described hereinabove.

According to certain embodiments, selecting plants resistant to a rust disease is performed by inoculating the plants with the respective fungus and selecting phenotypically resistant plants. According to certain exemplary embodiments, the inoculation and selection is performed at the seedling stage of the plants.

The respective fungus is as described hereinabove. According to certain embodiments, the method comprises selecting a Triticeae plant having an enhanced tolerance and/or resistance to leaf rust disease and stripe rust disease. According to these embodiments, the plants are inoculated with Pt and Pst.

According to additional or alternative embodiments, selecting plants resistant to a rust disease is performed by detecting the presence of the heterologous polynucleotide within the genetic material of the at least one cell of the Triticeae plant. Any method as is known in the art can be used to detect the heterologous polynucleotide. According to certain embodiments, detection is performed by identifying at least one sequence-specific probe that specifically hybridizes under stringent conditions to a nucleic acid sequence having at least 88% identity to the nucleic acid sequence set forth in SEQ ID NO:3 over its entire length. According to certain exemplary embodiments, the at least one sequence-specific probe specifically hybridizes under stringent conditions to the nucleic acid sequence set forth in SEQ ID NO:3.

According to certain embodiments, detection of the resistance-conferring polynucleotide is performed by detecting, in a genetic material obtained from the plant the presence of at least one nucleic acid marker amplified by a pair of primers. According to certain exemplary embodiments, the pair of primers is selected from the group consisting of a pair comprising SEQ ID NO:14 and SEQ ID NO:15; a pair comprising SEQ ID NO:16 and SEQ ID NO:17; a pair comprising SEQ ID NO:42 and SEQ ID NO: 43; a pair comprising SEQ ID NO:44 and SEQ ID NO:45; a pair comprising SEQ ID

NO: 46 and SEQ ID NO:47; a pair comprising SEQ ID NO:48 and SEQ ID NO:49; a pair comprising SEQ ID NO:50 and SEQ ID NO:51; a pair comprising SEQ ID NO:52 and

SEQ ID NO:53; a pair comprising SEQ ID NO:54 and SEQ ID NO:55; a pair comprising SEQ ID NO:56 and SEQ ID NO:57; a pair comprising SEQ ID NO:58 and SEQ ID NO: 59; and a pair comprising SEQ ID NO:60 and SEQ ID NO:61. Each possibility represents a separate embodiment of the present invention.

The rust disease and the pathogenic fungi causing same are as described hereinabove.

According to yet further aspects, the present invention provides an isolated polynucleotide encoding a polypeptide having at least 80% identity to the nucleic acid sequence set forth in SEQ ID NO:1, wherein the polynucleotide, when expressed in a Triticeae plant cell is capable of conferring or enhancing tolerance and/or resistance of the plant to at least one rust disease.

According to certain embodiments, the isolated polynucleotide comprises a nucleic acid sequence having at least 70% identity to the nucleic acid sequence set forth in SEQ ID NO: 6 over the entire length of the polynucleotide.

According to certain embodiments, the isolated polynucleotide comprises a nucleic acid sequence having at least 70% identity to the nucleic acid sequence set forth in SEQ ID NO: 6 over introns comprised within the sequence at positions having SEQ ID NO:7, SEQ ID NO:8 and SEQ ID NO:9; and at least 88% identity over exons comprised within said sequence having SEQ ID NO:10, SEQ ID NO:11, SEQ ID NO:12 and SEQ ID NO: 13.

According to certain embodiments, the isolated polynucleotide comprises a nucleic acid sequence having at least 89%, at least 90%, at least 91%, at least 92%, at least 93%, at least 94%, at least 95%, at least 96%, at least 97%, at least 98%, or at least 99% identity to exons comprised within the nucleic acid sequence set forth in SEQ ID NO:6. According to certain exemplary embodiments, the isolated polynucleotide comprises the nucleic acid sequence set forth in SEQ ID NO:6.

According to some embodiments, the isolated polynucleotide comprises a nucleic acid sequence having at least 88% identity to the nucleic acid sequence set forth in SEQ ID NO: 3 over its entire length.

According to some embodiments, the isolated polynucleotide comprises a nucleic acid sequence having at least at least 89%, at least 90%, at least 91%, at least 92%, at least 93%, at least 94%, at least 95%, at least 96%, at least 97%, at least 98%, or at least 99% identity to the nucleic acid sequence set forth in SEQ ID NO:3 over its entire length. According to certain additional exemplary embodiments, the isolated polynucleotide comprises the nucleic acid sequence set forth in SEQ ID NO:3.

According to certain embodiments, the rust disease is a leaf rust disease. According to certain exemplary embodiments, the leaf rust disease is caused by the fungus Puccinia triticina (Pt). According to certain embodiments, the rust disease is stripe rust disease. According to certain embodiments, the stripe rust disease is caused by the fungus Puccinia striiformis (Pst).

According to additional aspects, the present invention provides a nucleic acid construct comprising the isolated polynucleotides according to some embodiments of the invention, further comprising at least one operably linked regulatory element. According to certain embodiment, the regulatory element is selected from the group consisting of a promoter, an enhancer and a transcription termination sequence. The regulatory element, particularly the promoter and/or the terminator, can be endogenous or heterologous to the plant comprising the nucleic acid construct. According to certain embodiments, the promoter is heterologous to the wheat plant. According to some embodiment, the heterologous promoter is derived from Ae. longissima. According to certain exemplary embodiments, the promoter is the native promoter deriving the expression of the rust-disease tolerance/resistance conferring gene in Ae. longissima, comprising a nucleic acid sequence having at least 90% identity to the nucleic acid sequence set forth in SEQ ID NO: 4. According to some embodiment, the heterologous transcription termination sequence is derived from Ae. longissima. According to certain exemplary embodiments, the transcription termination sequence is the native sequence terminating the transcription of the rust-disease tolerance/resistance conferring gene in Ae. longissima, comprising a nucleic acid sequence having at least 90% identity to the nucleic acid sequence set forth in SEQ ID NO:5. According to certain embodiments, the DNA construct is a plant expression vector. According to some embodiments, the DNA construct or expression vector further comprises at least one selection marker.

According to certain exemplary embodiments, the Triticeae plant is a wheat plant or a barely plant.

The at least one rust disease and the fungi causing same are as described hereinabove.

According to yet further aspect, the present invention provides an isolated promoter sequence capable of deriving the expression of a polynucleotide within a cell of a Triticeae plant, the isolated promoter comprising a nucleic acid sequence having at least 90% identity to the nucleic acid sequence set forth in SEQ ID NO:4. According to certain currently exemplary embodiments, the isolated promoter comprising the nucleic acid sequence set forth in SEQ ID NO:4. According to some embodiments, the promoter is capable of driving the expression of a polynucleotide capable of conferring and/or enhancing the resistance and/or tolerance of a Triticeae plant to at least one rust disease.

The Triticeae plant and the rust disease are as described hereinabove.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows distribution of leaf rust resistance across the 138 Ae. longissima accessions.

Infection types (IT) are: 1=complete resistance, 9=fully susceptible. IT of the two isolates were correlated with an r=0.9, p<0.001

FIG. 2 shows the association between the LR phenotype (resistance to Lr strains, using Puccinia triticina race #12460) and the nucleotide-binding and leucine-rich repeat (NLR) contigs. Each dot column on the x axis represents an NLR contig from the RenSeq assembly of AEG-6782. Each dot on the y axis represents one or more RenSeq k-mers associated with resistance across the diversity panel. The association score is defined as the negative log of P value obtained using general model line (GLM). Dot size is proportional to the number of k-mers associated with resistance.

FIG. 3 presents a schematic representation of the gene constructs used in transformation of wheat cv. Fielder. Grey rectangles denote UTR regions, white rectangles denote exons, black lines (bottom scheme) denote the partial or full intronic sequences that were used.

FIG. 4 is a schematic presentation of the pRC3646 Lr_NLP: : Lr_gDNA: : Lr_NLT binary vector used for wheat transformation. The vector is based on the pRC3646 backbone and includes sequences of endogenous promoter, genomic sequence with short introns and endogenous terminator sequence of the Lr/Yr548 gene (SEQ ID NO:67).

FIG. 5 is a schematic presentation of the pRC3646 Lr_NLP: : Lr_cDNA: : Lr_NLT binary vector used for wheat transformation. The vector is based on the pRC3646 backbone and includes sequences of endogenous promoter, coding sequence and endogenous terminator sequence of the Lr/Yr548 gene (SEQ ID NO:68).

FIG. 6 is a schematic presentation of the pVS1-VIR helper plasmid.

FIG. 7 shows position comparison in the two versions of Ae. longissima genome assemblies.

FIG. 8 shows Lr/Yr548 copy number analysis in the T0 transgenic lines by ddPCR.

FIG. 9 shows reaction of transgenic lines to infection with Pt and Pst isolates, scale bar—2 mm.

FIG. 10 shows relative transcript levels at seedling stage of the Ae. longissima Lr/Yr548 gene in cDNA (T-c) and gDNA (T-g) transgenic wheat vs cv. Fielder and in Ae. longissima #AEG-6782-2. The D6PK-like gene (XM_044524929) was used as an internal control.

FIG. 11 demonstrates relative transcript levels of the Lr/Yr548 gene in cDNA (T-c) transgenic wheat vs cv. Fielder at the seedling stage. The D6PK-like gene (XM_044524929) was used as an internal control.

FIG. 12 demonstrates Relative transcript levels of the Lr/Yr548 gene in Pt infected vs non-infected (mock) AEG-548-4 plants.

FIG. 13 demonstrates Relative transcript levels of the Lr/Yr548 gene in seedling vs adult plants of line transgenic T-g event A.

FIG. 14 demonstrates the reaction of adult transgenic line T-g event A to infection with Pt isolate #12460. Scale-1 mm.

FIG. 15A shows pustules development caused by Puccinia on seedling (Pt, 7 dpi, Pst, 14 dpi) and adult (Pt, 18-20 dpi, Pst, 17-20 dpi) leaves of wheat cv. Fielder and derived transgenic plants. Scale bar=2 mm.

FIG. 15B shows light (upper panel) and fluorescent (lower panel) micrographs of leaf segments of wheat seedling inoculated with Pt and Pst. Upper level shows that the Lr/Yr548 transgenic seedlings developed chlorotic halos, that occasionally produce micro pustules (Pt, 7 dpi, Pst, 14 dpi). Bars=200 μm. Lower level shows leaves that were stained with WGA-FITC. Spores of both Pt and Pst germinated and penetrated the epidermis layer of the Lr/Yr548 transgenic plants but progressed slower and developed smaller colonies within the leaf compared with infection of cv. Fielder plants. Scale bar=200 μm.

FIG. 16 shows seedling reaction to infection with Pt isolate #526-24, 7dpi, after staining with WGA-FITC. Left panel-susceptible Ae. longissima AEG-1513-5. Right panel—resistant Ae. longissima AEG-678-2.

FIG. 17 shows the reaction of Ae. tauschii accessions to Pt #526-24, and PCR test for presence of RGA1 homologue. Scale—1 mm.

FIG. 18 shows phylogenetic relationship of the Lr/Yr548 gene and cloned NLR disease resistance genes from cereal crops and wild grasses based on full length Lr/Yr548 protein sequence (FIG. 18A) or CC domain sequence (FIG. 18B).

FIG. 19 shows phylogenetic relationship of Lr/Yr 548 and other disease resistance NLR genes from cereal crops and wild grasses based on: LRR domain (FIG. 19A) and NBS domain (FIG. 19B).

### DETAILED DESCRIPTION OF THE INVENTION

The present invention discloses novel genes that confer or enhance tolerance and/or resistance of Triticeae plants towards at least one rust disease, particularly wheat leaf rust disease and/or wheat stripe rust disease, (the latter also known as yellow rust disease). According to certain exemplary embodiments, the gene enhance tolerance and/or resistance towards both leaf rust and stripe rust diseases. The wheat rust diseases are caused by races of the fungi genus Puccinia. In an independent work of the inventors of the present invention and co-workers, a homologous gene has been identified in Aegilops sharonensis. The invention further provides Triticeae plants comprising within at least part of its cells heterologous polynucleotide comprising the tolerance and/or resistance-conferring nucleic acid sequence that show enhanced tolerance and/or resistance to the fungi. The invention further provides methods of producing and selecting the wheat plants having enhanced tolerance and/or resistance to the at least one rust disease. The present invention further provides methods for controlling a rust disease in agricultural production of Triticeae plant crop.

### Definitions

The term “plant” is used herein in its broadest sense. It also refers to a plurality of plant cells that are largely differentiated into a structure that is present at any stage of a plant's development. Such structures include, but are not limited to, a root, stem, shoot, leaf, flower, petal, fruit, etc. According to certain exemplary embodiments, the term “wheat plant” refers to Triticum turgidum subsp. durum (tetraploid wheat=macaroni wheat) and T. aestivum subsp. aestivum (hexaploid wheat=bread wheat=common wheat) of the tribe Triticeae, family Poaceae (Gramineae).

The term “cultivar” (abbreviation cv.) is used herein to denote a plant having a biological status other than a “wild” status, which “wild” status indicates the original non-cultivated or natural state of a plant or accession. The term “cultivar” (for cultivated plants) includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, and advanced/improved cultivar. The term as used herein includes registered as well as non-registered lines. Examples of cultivars include such cultivated varieties that belong to the species Triticum turgidum including, but not limited to cultivar Svevo, and Triticum aestivum, including, but not limited to cultivars “Fielder”, Chinese Spring” (CS) and “Galil”.

The terms Aegilops longissima and Ae. longissima are used herein interchangeable and refer to a wild plant of the genus Aegilops, belonging to the plant family Poaceae (sub-family Pooideae). According to certain embodiments, certain accession within Ae. longissima populations are resistant to at least one rust disease, particularly to leaf rust disease and/or stipe rust disease.

The terms “resistant” and “resistance” as used herein refer to the ability of a plant to restrict the growth and development of a specified pest or pathogen and/or the damage they cause when compared to susceptible plant grown under similar environmental conditions and pest or pathogen pressure. The terms encompass both partial and full resistance to infection. A rust-resistant plant may either be fully resistant or have low levels of susceptibility to infection by the fungus Puccinia, particularly by Puccinia triticina, and/or Puccinia striiformis.

The terms “tolerant” and “tolerance” are used herein to indicate a phenotype of a plant wherein at least some of the disease-symptoms remain absent upon exposure of said plant to an infective dose of a pathogen, particularly fungi, whereby the presence of the pathogen can be established, at least under some culture conditions. Tolerant plants are therefore free of the pathogen or are symptomless carriers of the pathogen, particularly the fungi.

The terms “susceptible” and “susceptibility” as used herein refer to the inability of a plant to restrict the growth and development of a specified pest or pathogen; a susceptible plant displays the detrimental symptoms linked to the pathogen infection, particularly fungi infection. A rust-susceptible wheat plant may be either non-resistant, have low levels of resistance or non-tolerant to these fungi. Stripe rust (also designated yellow rust, caused by the fungus Puccinia striiformis) and leaf rust (caused by Puccinia triticina) are two devastating wheat diseases causing enormous annual yield losses. The fungal pathogens are changing frequently, giving rise to new virulent types (races) and thus overcoming resistance genes that have been developed. Consequently, the primary wheat gene pool is becoming exhausted and new resistance genes are required. Wild relatives of wheat are yet a relatively untapped resistance gene pool.

Accordingly, “conferred tolerance and/or resistance to rust disease(s)” or “enhanced tolerance and/or resistance to a rust disease(s)” refer to a phenotype in which a plant, a wheat plant according to the present invention, has greater health, growth, multiplication, fertility, vigor, strength (e.g., stem strength and resistance), yield, or less severe symptoms associated with infection of the pathogenic fungus causing the rust disease compared to a wheat plant that does not have enhanced tolerance and/or resistance to the pathogen. According to certain embodiments, of the invention, the wheat plant that does not have enhanced tolerance and/or resistance to the pathogen is lacking the heterologous polynucleotide(s) of the invention. Where a plant is tested for resistance, a control plant is used to assess the degree of the plant resistance. According to certain embodiments of the present invention, the control plant is a plant not manipulated to comprise within its cells the resistance-conferring or enhancing polynucleotide of the invention. The control plant typically, but not necessarily, has the same genetic background as the examined plant. The enhancement can be manifested as an increase of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more in health, growth, multiplication, fertility, vigor, strength, or yield, as compared to a control plant. The enhancement can be a decrease of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, or 100% in the symptoms associated with the fungi infection as compared to the control plant. According to certain exemplary embodiments, the examined plant and the control plant are grown under the same conditions.

As used herein the term “polynucleotide” refers to a single or double stranded nucleic acid sequence which is isolated and provided in the form of an RNA sequence, a complementary polynucleotide sequence (cDNA), a DNA sequence and/or composite polynucleotide sequences (e.g., a combination of the above).

As used herein, the term “contig” refers to a set of overlapping DNA segments that together represent a consensus region of DNA.

The terms “protein” and “polypeptide” are used interchangeably herein to designate a series of amino acid residues, connected to each other by peptide bonds between the alpha-amino and carboxyl groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “isolated” refers to at least partially separated from the natural environment e.g., from a plant cell. According to certain embodiments, the polynucleotides of the invention are isolated from Ae. longissima plant cells.

The term “heterologous” with reference to a polynucleotide as is used herein refers to a sequence that is not naturally found in the plant, specifically the wheat plant, and has been artificially introduced into the plant.

The term “heterozygous” as is used herein means a genetic condition existing when different alleles (forms of a given gene, genetic determinant, or sequences) reside at corresponding loci on homologous chromosomes.

The term “homozygous” as is used herein, means a genetic condition existing when identical alleles (forms of a given gene, genetic determinant, or sequences) reside at corresponding loci on homologous chromosomes.

The terms “genetic engineering”, “transformation” and “genetic modification” are all used herein for the transfer of isolated and cloned genes, genetic determinants or polynucleotides into the DNA, usually the chromosomal DNA or genome, of another plant, or to the modification of a gene within the plant genome.

As used herein, the term “plant part” typically refers to a part of the wheat plant. Examples of plant parts include, but are not limited to, pollen, ovules, leaves, embryos, roots, root tips, anthers, flowers, fruits, stems shoots, and seeds. The term further refers to single cells and cell tissues such as plant cells that are intact in plants, protoplasts, cell clumps, tissue cultures and calli from which wheat plants can be regenerated, that are derived from pollen, ovules, leaves, embryos, roots, root tips, anthers, flowers, fruits, stems shoots, and seeds.

As used herein, the term “population” refers to a genetically heterogeneous collection of plants sharing a common genetic derivation.

The present invention is based in part of the discovery of a novel gene within the genome of Ae. Longissima, designated herein LrYr5458, associated with the Ae. Longissima tolerance/resistance to rust diseases, particularly to leaf rust disease and stripe rust disease. Unexpectedly, the gene, belonging to the family of Nucleotide-binding site leucine-rich repeat (NLR) disease resistance genes, encodes for a protein conferring resistance to two types of rust against both Puccinia triticina (Pt) and P. striiformis f. sp. tritici (Pst) that cause leaf and stripe rust, respectively. According to certain aspects, the present invention provides a Triticeae plant comprising at least one cell comprising a heterologous polynucleotide encoding a polypeptide having at least 80% identity to the amino acid sequence set forth in SEQ ID NO:1, wherein the heterologous polynucleotide is capable of conferring or enhancing tolerance and/or resistance of the plant to at least one of rust disease.

According to certain embodiments, the heterologous polynucleotide comprises a nucleic acid sequence encoding a polypeptide having at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO:1. According to certain embodiments, the encoded polypeptide comprises the amino acid sequence set forth in SEQ ID NO:1. According to certain embodiments, the encoded polypeptide consists of SEQ ID NO:1.

As used herein, “sequence identity” or “identity” in the context of two polypeptide or nucleic acid sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. (Amino acid substitution matrices from protein blocks. Proc. Natl. Acad. Sci. U.S.A. 89(22), 10915-9, 1992).

Identity (e.g., percent homology) can be determined using any homology comparison software, including for example, the BlastN, BlastX or Blastp software of the National Center of Biotechnology Information (NCBI) such as by using default parameters. A widely used and accepted computer program for performing sequence alignments is CLUSTALW v1.6 (Thompson, et al. Nucl. Acids Res., 22:4673-4680, 1994).

According to some embodiments of the invention, the identity is a global identity, i.e., over the entire nucleic acid sequences of the invention and not over portions thereof. According to some embodiments of the invention, the identity is a partial identity, i.e., over fragment or fragments of the nucleic acid sequences of the invention and not over the entire sequence, as described herein.

According to further aspects, the present invention provides isolated polynucleotide encoding a polypeptide having at least 80% identity to the amino acid sequence set forth in SEQ ID NO:1, wherein the polynucleotide, when expressed in a Triticeae plant cell is capable of conferring or enhancing tolerance and/or resistance of the plant to at least one rust disease.

According to some embodiments, the encoded protein comprises an amino acid sequence having at least 81%, at least 82%, at least 83%, at least 84%, at least 85%, at least 86%, at least 87%, at least 88%, or at least 89% identity, at least 90% identity, at least 91%, at least 92%, at least 93%, at least 94%, at least 95%, at least 96%, at least 97%, at least 98%, or at least 99% identity to the amino acid sequence set forth in SEQ ID NO: 1. Each possibility represents a separate embodiment of the present invention.

According to certain exemplary embodiments, the encoded protein comprises the amino acid sequence set forth in SEQ ID NO:1. According to further exemplary embodiments, the encoded protein consists of the amino acid sequence set forth in SEQ ID NO: 1.

In the course of the research of the present invention, the inventors have progressed to sequence the entire polynucleotide comprising the nucleic acid encoding the tolerance/resistance conferring polypeptide, including the sequences of all introns and exons as set forth in SEQ ID NO:6. SEQ ID NO:6 comprises SEQ ID NO:2 in its entirety.

According to certain embodiments, the isolated polynucleotide comprises a nucleic acid sequence having at least 70% identity to the nucleic acid sequence set forth in SEQ ID NO: 6 over the entire length of the polynucleotide.

According to certain embodiments, the heterologous polynucleotide comprises a nucleic acid sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identity to the nucleic acid sequence set forth in SEQ ID NO:6 over introns comprised within the sequence and having SEQ ID NO:7, SEQ ID NO:8 and SEQ ID

NO: 9; at least 88%, at least 89%, at least 90%, at least 91%, at least 92%, at least 93%, at least 94%, at least 95%, at least 96%, at least 97%, at least 98%, at least 99% or more identity over of exons comprised within said sequence and having SEQ ID NO:10, SEQ ID NO: 11, SEQ ID NO:12 and SEQ ID NO:13. Each possibility represents a separate embodiment of the present invention.

According to certain embodiments, the intron having SEQ ID NO:7 is located at positions 295-5,327 of SEQ ID NO:6. According to certain embodiments, the intron having SEQ ID NO:8 is located at positions 6,484-13,006 of SEQ ID NO:6. According to certain embodiments, the intron having SEQ ID NO:9 is located at positions 16,008-16,124 of SEQ ID NO:6.

According to certain embodiments, the exon having SEQ ID NO:10 is located at positions 1-294 of SEQ ID NO:6. According to certain embodiments, the exon having

SEQ ID NO:11 is located at positions 5,328-6,483 of SEQ ID NO:6. According to certain embodiments, the exon having SEQ ID NO:12 is located at positions 13,007-16,007 of SEQ ID NO:6. According to certain embodiments, the exon having SEQ ID NO:13 is located at positions 16,125-16,207 of SEQ ID NO:6.

According to certain embodiments, the isolated polynucleotide comprises the nucleic acid sequence set forth in SEQ ID NO:6. According to certain embodiments, the isolated polynucleotide comprises a nucleic acid sequence encoding a polypeptide having the amino acid sequence set forth in SEQ ID NO: 1, the nucleic acid sequence consisting of SEQ ID NO:6.

According to yet further embodiments, the isolated polynucleotide comprises a nucleic acid sequence having at least 88%, at least 89%, at least 90%, at least 91%, at least 92%, at least 93%, at least 94%, at least 95%, at least 96%, at least 97%, at least 98%, or at least 99% identity to the nucleic acid sequence set forth in SEQ ID NO:3 over its entire length. According to certain embodiments, the heterologous polynucleotide comprises the nucleic acid sequence set forth in SEQ ID NO:3.

According to certain aspects, the present invention provides a pair of primers capable of identifying a polynucleotides having at least 88% identity to the nucleic acid sequence set forth in SEQ ID NO:3. According to certain exemplary embodiments, the pair of primers is selected from the group consisting of a pair comprising SEQ ID NO: 14 and SEQ ID NO:15; a pair comprising SEQ ID NO:16 and SEQ ID NO:17; a pair comprising SEQ ID NO:42 and SEQ ID NO:43; a pair comprising SEQ ID NO:44 and SEQ ID NO:45; a pair comprising SEQ ID NO:46 and SEQ ID NO:47; a pair comprising SEQ ID NO:48 and SEQ ID NO:49; a pair comprising SEQ ID NO:50 and SEQ ID NO: 51; a pair comprising SEQ ID NO:52 and SEQ ID NO:53; a pair comprising SEQ ID NO: 54 and SEQ ID NO:55; a pair comprising SEQ ID NO:56 and SEQ ID NO:57; a pair comprising SEQ ID NO:58 and SEQ ID NO:59; and a pair comprising SEQ ID NO:60 and SEQ ID NO:61. Each possibility represents a separate embodiment of the present invention.

According to certain exemplary embodiments, the pair of primers comprises SEQ ID NO: 14 and SEQ ID NO:15.

According to certain additional or alternative embodiments, the pair of primers comprises \ SEQ ID NO: 16 and SEQ ID NO:17.

According to additional aspects, the present invention provides a nucleic acid construct comprising the isolated polynucleotide of the invention, further comprising at least one regulatory element for directing transcription of the nucleic acid sequence in the host plant cell, particularly in wheat plant cell.

According to certain embodiments, the regulatory element is selected from the group consisting of an enhancer, a promoter, a transcription termination sequence, and the like. According to some embodiments of the invention, the regulatory sequence is operably linked to the isolated polynucleotide.

A nucleic acid sequence (particularly a coding nucleic acid sequence) is “operably linked” to a regulatory sequence (e.g., promoter) if the regulatory sequence is capable of exerting a regulatory effect on the coding sequence linked thereto.

According to certain embodiments, the nucleic acid construct is an expression vector comprising a promoter operably linked to the polynucleotide of the invention.

As used herein, the term “promoter” refers to a region of DNA placed upstream of the transcriptional initiation site of a gene to which RNA polymerase binds to initiate transcription of RNA. The promoter controls where (e.g., which portion of a plant) and/or when (e.g., at which stage or condition in the lifetime of an organism or a cell thereof) the gene is expressed.

According to some embodiments of the invention, the promoter is heterologous to the isolated polynucleotide and/or to the host cell.

As used herein the phrase “heterologous promoter” refers to a promoter from a different species or from the same species but from a different gene locus as of the isolated polynucleotide sequence.

According to additional or alternative embodiments of the invention, the promoter forming part of the nucleic acid construct is the promoter driving the expression of the tolerance/resistance conferring gene in Ae. Longissimi. According to these embodiments, the promoter comprises a nucleic acid sequence having at least 90% identity to the nucleic acid sequence set forth in SEQ ID NO:4. According to certain currently exemplary embodiments, the promoter comprises the nucleic acid sequence set forth in SEQ ID NO: 4.

Any suitable promoter sequence can be used by the nucleic acid construct of the present invention. Preferably the promoter is selected from the group consisting of a constitutive promoter, a tissue-specific, or biotic-stress specific promoter, particularly promoters inducible by fungi infection. According to some embodiments of the invention, the promoter is a plant promoter suitable for expression of the isolated polynucleotide of the invention in a wheat plant cell. An exemplary promoter to be used in wheat plant is Lr21 promoter (Huang, Li, et al., 2003, Genetics 164.2:655-664).

According to additional or alternative embodiments of the invention, the promoter forming part of the nucleic acid construct is the promoter driving the expression of the tolerance/resistance conferring gene in Ae. Longissimi. According to these embodiments, the promoter comprises a nucleic acid sequence having at least 90%, at least 91%, at least 92%, at least 93%, at least 94%, at least 95%, at least 96%, at least 97% m at least 98% or at least 99% identity to the nucleic acid sequence set forth in SEQ ID NO:4. According to certain currently exemplary embodiments, the promoter comprises the nucleic acid sequence set forth in SEQ ID NO:4. According to some embodiments, the promoter consists of the nucleic acid sequence set forth in SEQ ID NO:4.

According to certain embodiments, the nucleic acid construct further comprises a termination sequence located 3′ to the coding sequence. According to certain embodiments, the termination sequence is derived from Aegilops longissima. According to certain exemplary embodiments, the termination sequence is the natural terminator of the tolerance/resistance conferring gene in Ae. longissima. According to these embodiments, the terminator comprises a nucleic acid sequence having at least 90%, at least 91%, at least 92%, at least 93%, at least 94%, at least 95%, at least 96%, at least 97% m at least 98% or at least 99% identity to the nucleic acid sequence set forth in SEQ ID NO: 5. According to certain currently exemplary embodiments, the termination sequence comprises the nucleic acid sequence set forth in SEQ ID NO:5. According to some embodiments, the termination sequence consists of the nucleic acid sequence set forth in SEQ ID NO:5.

The nucleic acid construct of the present invention can further comprise at least one marker (reporter) gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the markers gene). Many commonly used selectable marker genes for plant transformation are known in the art, and include, for example, genes that code for enzymes that metabolically detoxify a selective chemical agent which may be an antibiotic or an herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art. The construct according to the present invention being a transformation vector, an expression vector or a combination thereof can be, for example, plasmid, a bacmid, a phagemid, a cosmid, a phage, a virus or an artificial chromosome.

The polynucleotides of the invention and construct comprising same can be chemically synthesized by any method as is known in the Art.

The nucleic acid construct comprising the polynucleotide conferring or enhancing tolerance and/or resistance to at least one rust disease as disclosed herein may be used for the production of a wheat plant having enhanced tolerance and/or resistance to the at least one rust disease. According to certain embodiments, the tolerance/resistance-conferring polynucleotide or the construct comprising same is introduced into a susceptible wheat plant, typically to a wheat cultivar used in agriculture.

The tolerance/resistance conferring nucleic acid sequence may be introduced to a recipient wheat plant by any method as is known to a person skilled in the art. According to certain embodiments, the isolated polynucleotide or the construct comprising same according to the teachings of the invention can be introduced by transformation. Transformation is optionally followed by selection of offspring plants comprising the resistance-conferring sequence and exhibiting resistance to the fungal diseases leaf rust and/or stripe rust.

According to additional aspects, the present invention provides a method for producing a Triticeae plant having enhanced resistance to at least one rust diseases, the method comprises introducing into at least one cell of a Triticeae plant susceptible to the at least one rust diseases a heterologous polynucleotide encoding a polypeptide having at least 80% identity to the amino acid sequence set forth in SEQ ID NO:1, thereby producing a wheat plant having enhanced tolerance and/or resistance to said at least one rust diseases compared to a corresponding control plant.

The heterologous polynucleotide is as described hereinabove. Methods for transforming a plant cell with polynucleotides and/or constructs comprising same according to the present invention are known in the art. As used herein the term “transformation” or “transforming” describes a process by which a foreign nucleic acid sequence, such as a vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to typical embodiments the nucleic acid sequences of the present invention are stably transformed into a plant cell resulting in a cell comprising within its genome the heterologous polynucleotide.

There are various methods of introducing foreign nucleic acid sequences into both monocotyledonous and dicotyledonous plants (for example, Potrykus I. 1991. Annu Rev Plant Physiol Plant Mol Biol 42:205-225; Shimamoto K. et al., 1989. Nature 338:274-276).

The principal methods of the stable integration of exogenous DNA into plant genomic DNA includes two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. Agrobacterium mediated transformation protocols for wheat are known to a person skilled in the art. High efficiency transformation of wheat embryos mediated by Agrobacterium tumefaciens is described by Ishida et al. (Ishida Y., et al. In: Ogihara Y., Takumi S., Handa H. (eds) Advances in Wheat Genetics: From Genome to Field. Springer, Tokyo. DOI 10.1007/978-4-431-55675-6_18).

Direct nucleic acid transfer: There are various methods of direct nucleic acid transfer into plant cells, however, not all are applicable in Triticeae. A directed transformation method that any be used is microparticle bombardment, in which the nucleic acid is adsorbed on microprojectiles such as magnesium sulfate crystals or tungsten particles, and the microprojectiles are physically accelerated into cells or plant tissues.

According to other embodiments, the tolerance/resistance conferring polynucleotides of the present invention can be introduced into the genome of at least one cell of a susceptible Triticeae plant using the techniques of genome editing. Genome editing is a reverse genetics method which uses artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single-or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

According to certain additional aspects, the present invention provides a method for selecting a Triticeae plant having an enhanced tolerance and/or resistance to at least one rust diseases, comprising the steps of:
a. providing a plurality of plants each comprising at least one cell comprising a heterologous polynucleotide conferring or enhancing tolerance and/or resistance to the Triticeae cultivar towards at least one rust disease, wherein the heterologous polynucleotide encodes a polypeptide comprising an amino acid sequence having at least 80% identity to the amino acid sequence set forth in SEQ ID NO:1; and
b. selecting plants showing an enhanced resistance to said at least one rust diseases compared to a control plant or to a pre-determined resistance score value;
thereby selecting a plant having enhanced resistance to said at least one rust diseases.

According to certain embodiments, selecting plants resistant to a rust disease is performed by inoculating the plants with the respective fungus and selecting phenotypically resistant plants. According to certain exemplary embodiments, the inoculation and selection is performed at the seedling stage of the plants.

According to additional or alternative embodiments, selecting plants resistant to a rust disease is performed by detecting the presence of the heterologous polynucleotide within the at least one cell of the Triticeae plant. Any method as is known in the art can be used to detect the heterologous polynucleotide. According to certain embodiments, detection is performed by identifying at least one sequence-specific probe that specifically hybridizes under stringent conditions to a nucleic acid sequence having at least 70% identity to the nucleic acid sequence set forth in SEQ ID NO:6 over introns comprised within the sequence SEQ ID NO:7, SEQ ID NO:8 and SEQ ID NO:9; and at least 88% identity over exons comprised within said sequence having SEQ ID NO:10, SEQ ID NO:11, SEQ ID NO: 12 and SEQ ID NO: 13.

According to certain exemplary embodiments, the at least one sequence-specific probe specifically hybridizes under stringent conditions to the nucleic acid sequence set forth in SEQ ID NO:6.

According to additional embodiments, detection is performed by amplifying at least one marker located within a polynucleotide having at least 88% identity to the nucleic acid sequence set forth in SEQ ID NO:3. According to certain exemplary embodiments, the pair of primers is selected from the group consisting of a pair comprising pair comprising SEQ ID NO:14 and SEQ ID NO:15; a pair comprising SEQ ID NO:16 and SEQ ID NO:17; a SEQ ID NO:42 and SEQ ID NO:43; a pair comprising SEQ ID NO:44 and SEQ ID NO:45; a pair comprising SEQ ID NO:46 and SEQ ID NO:47; a pair comprising SEQ ID NO:48 and SEQ ID NO:49; a pair comprising SEQ ID NO:50 and SEQ ID NO:51; a pair comprising SEQ ID NO:52 and SEQ ID NO:53; a pair comprising SEQ ID NO:54 and SEQ ID NO:55; a pair comprising SEQ ID NO:56 and SEQ ID NO: 57; a pair comprising SEQ ID NO:58 and SEQ ID NO:59; and a pair comprising SEQ ID NO: 60 and SEQ ID NO:61. Each possibility represents a separate embodiment of the present invention.

Rust diseases are caused by fungi of the genus Puccinia. Multiple disease resistance is usually conferred by non-NLR resistance genes with a general function, such as the rice broad-spectrum resistance genes Pi21 and Ptr, that encode for a proline-rich protein (Fukuoka S et al., 2009. Science 325(5943) 998-1001) and a protein with four Armadillo repeats (Zhao H et al., 2018. Nature communications. 9(1):2039) respectively. Another example are the wheat pleiotropic effect resistance genes Lr34 and Lr67 that encode for an ABC transporter (Krattinger S G et al., 2009. Science 323(5919):1360-3) and a hexose transporter (Moore J W et al., 2015. Nature genetics. 2015 47(12):1494-8), respectively. Although rare, there have been several reports of resistance conferred by a single NLR resistance gene against taxonomically distant pathogens. The RRS1 and RPS4 NLR genes in A. thaliana function cooperatively to confer resistance against three bacterial pathogens—Pseudomonas syringae, Ralstonia solanacearum, and Xanthomonas campestris, and one fungal pathogen Colletotrichum higginsianum (Narusaka M et al., 2014. Plant signaling & behavior 9 (7): e29130). Likewise, the Mi NLR gene in tomato confers resistance to three types of pests, the root knot nematode Meloidogyne spp., potato aphid Macrosiphum euphorbiae, and the whitefly Bemisia argentifolii (Milligan S B et al., 1998. The Plant Cell 10 (8): 1307-19; Rosi M et al., 1998. Proceedings of the National Academy of Sciences 95 (17): 9750-4; Nombela G et al., 2003. Molecular Plant-Microbe

Interactions. 16 (7): 645-649) and the Roq1 gene confers resistance to the phytopathogenic bacteria Xanthomonas and Pseudomonas in Nicotiana benthamiana (Schultink A et al., 2017. The Plant Journal. 92(5):787-95; Li W et al., 2020. Annu. Rev. Plant Biol. 2020 71:575-603). In grasses, the only examples of dual resistance NLR genes are the barley Mla3 and Mla8. Both genes confer resistance to the host-adapted powdery mildew pathogen Blumeria graminis f. sp. hordei, but in addition, each of them confers resistance against a barley non-adapted pathogen: Mla3 confers resistance to the rice blast pathogen Magnaporthe oryzae by specific recognition of the Pw12 effector protein (Brabham H et al., 2022. BioRxiv doi.org/10.1101/2022.10.21.512921), and Mla8 has a coupled resistance against the wheat stripe rust pathogen Pst (Bettgenhaeuser J et al., 2021. Nat. Commun. 12:1-14).

There has been a single report in wheat of the Sr 15/Lr20 gene that confers resistance to both stem and stripe rust pathogens (McIntosh R A 1977. Nature of induced mutations affecting disease reaction in wheat. In: Induced mutations against plant diseases), however the gene has not been isolated, and its molecular nature is unknown. Marais et al. (Marais G F 2006. Biological Sciences 371 (1709): 20160026) reported on introgression of leaf and stripe rust resistance from Ae. sharonensis into chromosome 6A of bread wheat, however the resistance was associated with two separate genes Lr56 and Yr38, that segregated between offspring. Hence, the Lr/Yr548 gene reported here is a rare case and the first in wheat, of a cloned NLR gene that confers resistance to two different wheat pathogens.

Without wishing to be bound by any specific theory or mechanism of action, tolerance enhancement by the Lr/Yr548 gene dose not led to a hyper-sensitive response (HR), but rather slows down pathogen development and disease progression significantly. In many cases, incomplete/quantitative resistance is associated with plant age, namely genes that confer adult plant resistance (APR), which is manifested as partial resistance in adult plants but not in seedlings (Dinglasan E et al., 2022. Essays in Biochemistry 66(5):571-80; Sánchez-Martín J and Keller B 2021. Current Opinion in Plant Biology 62:102053; Ellis J G et al., 2014. Frontiers in plant science 5:641). Resistance levels were high and stable between seedlings and adult plants indicating that Lr/Yr548 is not an APR gene. Expression levels of the Lr/Yr548 gene were markedly increased in adult plants, however without a visible effect on resistance in adult plants compared with seedlings. A significant change in resistance at high temperatures was also not observed (not shown).

How the Lr/Yr548 protein confers resistance against two different pathogen species is unclear. Without wishing to be bound by any specific theory or mechanism of action, since resistance may be triggered by recognition of a fungal effector, the Lr/Yr548 protein possibly recognizes two alternative effectors, as in the case of the tobacco ROQ1 that recognizes different effectors from different bacterial species (Nakano M and Mukaihara T. 2019. Molecular Plant Pathology 20(9):1237-51). Alternatively, the two fungal species contain a common effector, as in the case of the Arabidopsis RRS1/RPS4 complex that recognizes the PopP2 and AvrRps4 bacterial effectors via a WRKY domain (Sarris P F et al., 2015. Cell 161(5):1089-100).

Phylogenetic analysis showed that the Lr/Yr548 gene is unique to Ae. longissima. The closest homologue, an uncharacterized RGAl gene with 87% identity to Lr/Yr548, was found in Ae. tauschii (Table 5 and Table 6 hereinbelow), encoding for the protein of SEQ ID NO:64, which is the donor of the hexaploid wheat D sub-genome and member of the same clade as Ae. longissima and Ae. sharonensis (Avni R et al., 2022. The Plant Journal January 8. doi.org/10.1111/tpj.15664). Ae. tauschii accessions were moderately resistant to the leaf and stripe rust isolates (not shown) regardless of the presence or absence of the RGAl gene, indicating that RGA1 is not a functional homologue of Lr/Yr548.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Plants and Pathogens

Ae. longissima diversity panel of 380 accessions (including Ae. longissima accession AEG-6782-2 (Avni et al., 2022, ibid) originated from 80 collection sites spanning the whole Ae. longissima geographic distribution in Israel. All accessions were self-fertilized for 3-5 generations, with bagged heads to prevent cross fertilization. Selection of accessions was done to maximize the geographic distribution of the accessions and to minimize the number of accessions originating from the same collection site.

Wheat cultivar Fielder (NSGC cltr. 17268) was obtained from USDA.

Puccinia triticina (Pt, Leaf rust) isolates #526-24 (race MFBKG), #12460 (race MCDTB), #12337 (race MFPPB) and Puccinia striiformis (Pst, Stripe rust) isolate #5006 (virulence formula Yr6,7,8,9,11,12,17,19,sk,18,A/Yr1,5,10,15,24,26,sp) were obtained from the stocks of the Institute for Cereal Crops Research (ICCR) at Tel Aviv University, Israel. The P. triticina nomenclature is according to updated code for North American differential hosts for P. triticina, USDA (www.ars.usda.gov/midwest-area/stpaul/cereal-disease-lab/docs/cereal-rusts/race-identification).

### Plant Inoculation and Phenotyping

Seedlings were tested and selected for leaf rust and stripe rust resistance according to Millet et al., 2014. Briefly, plants were sown and grown in small pots in a temperature-controlled greenhouse at 22±2° C. with photoperiod of 14/10 h. For most applications, seedlings were inoculated at the one leaf stage (7-10 days after planting, for leaf rust) or two-leaf stage (10-12 days after planting, for stripe rust) with a suspension of relevant Puccinia isolate urediniospores in a lightweight mineral oil (Soltrol® 170 Isoparaffin) and the oil on the inoculated plants was allowed to evaporate.

For gene expression studies, plants were inoculated by brushing leaves with fresh spores of the relevant Puccinia isolate. Following inoculation, plants were incubated for 24 h in a dew chamber (100% relative humidity) and then transferred to the greenhouse. For leaf rust inoculation, plants were incubated for 24 h in a dew chamber at 18° C., and grown in the greenhouse for 12-14 days at 22° C. For stripe rust inoculation, plants remained in a dew chamber for 24 h (12 h at 9° C. in the dark, followed by 12 h at 15° C. in the light) and were transferred to the growth chamber for 14-17 days at 15° C. For stepwise infection with both leaf and stripe rust, plants were first infected with a leaf rust isolate and incubated as described for 24 h in a dew chamber and for 4-6 days in the greenhouse, then infected with a stripe rust isolate, incubated in a dew chamber, and transferred to a growth chamber as described. For all types of inoculations, 3 replicates for each accession were tested Plants were scored for infection type (IT) 7-10 days after inoculation (Pt) and 14 days (Pst), on a standard 0 to 4 scale (McIntosh R A et al., 1995. Wheat rusts: an atlas of resistance genes. CSIRO publishing), converted to 1-9 scale for clarity. ITs of 0 to 2 (converted to 1-5) were considered indicative of a resistant response and 3 to 4 (converted to 6-9) as a susceptible response.

### Genomic DNA and RNA Isolation and Gene Expression Analysis

Genomic DNA was extracted from leaves by the CTAB method (Doyle J J and Doyle J L, 1987. Phytochemical bulletin 19(1):11-15). Total RNA was extracted according to Sharma et al., 2019. First-strand cDNA was synthesized using the RevertAid First Strand cDNA Synthesis Kit (ThermoFisher, Waltham, USA). All cDNA samples were diluted fourfold with water prior to use in quantitative real-time PCR (RT-qPCR). Primers for RT-qPCR were designed to anneal to different exons flanked by introns with amplicon lengths between 140 and 170 bp. Each reaction was run with at least three independent biological replicates. RT-qPCR analysis was performed with qPCRBIO SyGreen Mix (PCR Biosystems) on a PikoReal 96 Real-time PCR instrument (Thermo scientific). The quantitative RT-PCR data were analyzed by the comparative 2^{-ΔΔCt} method (Pfaffl M W, 2001. Nucleic acids research 29(9):e45). The D6PK-like gene (Triticum aestivum protein kinase, XM_044524929) was used as internal control. Statistical significance was analyzed by two-tailed Student's t-test. For measurement of the Lr/Yr548 gene expression in Ae. longissima accession AEG-6782-2, two-week-old seedings were inoculated with Pt #526-24 and Pst #5006. Single leaves from four individual plants were harvested for each treatment at the time of inoculation (T₀) and at 24, 48 and 72 hpi. To determine the level of gene expression at different growth stages of transgenic wheat plants cv. Fielder, leaves were harvested at seedling, booting and silk development stage.

Transgene copy number was performed as described in Collier et al., 2017. Briefly, gDNA was extracted from four-week old T₀ seedlings via CTAB as described above, digested with 20 U of HaeIII enzyme (NEB) in a 50 μl reaction volume for 16 hours at 37° C., and used as a template. ddPCR was performed via probe chemistry in a duplex assay for reference PINb (Giroux and Morris, 1997. Theoretical and applied genetics 95(5/6) 857-864; Li Z et al.,2004. Plant Molecular Biology Reporter 22:179-88) and target Bar genes. The Bar Probel (Table 1) was labeled with HEX at 5′, Iowa Black Hole Quencher at the 3′ end and with an internal ZEN quencher 9 nucleotides away from the 5′ end. The PINb Probe2 (Table 1) was labeled with FAM at the 5′end, with Iowa Black Hole Quencher at the 3′ end, and with internal ZEN quencher 9 nucleotides away from the 5′ end. Primers F12-R12 and F13-R13 were used for Bar and PINb genes respectively (Table 1). ddPCR reaction mixes were prepared according to the instructions in ddPCR Supermix for Probes (No dUTP) #1863024 kit (Biorad). Droplets were generated with a QX200 droplet generator (Biorad), the PCR was run in a C1000 (Biorad) deep-well thermal cycler. The fluorescence of the droplets was measured with a QX200 droplet analyzer (Biorad), and the results were evaluated with the BioRad Quantasoft Pro Software.

### NLR-Domain Annotation

To aid in the identification of functional resistance genes, the NLR-Annotator tool (Steuernagel B et al., 2020. Plant physiology 183 (2): 468-82) was used to annotate the intracellular immune repertoire. The NLR-Annotator pipeline is divided into three steps:

(1) dissection of genomic input sequence into overlapping fragments; (2) NLR-Parser, which creates an xml-based interface file; and (3) NLR-Annotator, which uses the xml file as input, annotates NLR loci, and generates output files based on coordinates and orientation of the initial input genomic sequence. The pipeline was run on the de novo assembled scaffolds and de novo assembled pooled transcripts, as well as on the AEG-548-4 transcripts, using the MEME suite v.5.0.1.1 (Bailey T L and Gribskov, M. 1998. Bioinformatics (Oxford, England 14(1):48-54), and otherwise default configuration.

### Phylogenetic Analysis

Using blastp (protein-protein BLAST), the amino acid sequence of Lr/Yr548 protein was searched for sequence similarities in the monocot taxonomy. BLAST was performed on a database of non-redundant protein sequences and the obtained results were filtered with a percentage identity of 60-90%. Moreover, the NLR cloned genes of wheat and barley (for examples, Lr10, SEQ ID NO:62 (wheat); Lr21, SEQ ID NO:63 (wheat); and |Lr22, SEQ ID NO:64 (Aegilops tauschii)) were retrieved from NCBI for phylogenetic analysis with Lr/Yr548. MEGA11 was used to perform evolutionary analyses on the entire protein as well as different domains. MUSCLE was used for multiple alignment, and the Neighbor-joining method was used to infer evolutionary tree. The evolutionary history of the taxa studied is represented by a bootstrap consensus tree inferred from 1000 replicates. The tree is drawn to scale, with branch lengths in the same units as those used to infer the phylogenetic tree.

### Phylogenetic Tree Construction

Each of the 246 AgrenSeq raw read samples was aligned to the Ae. longissima genome V2 using bwa mem algorithm (Li H 2013. Aligning sequence reads, clone sequences and assembly contigs with BWA-MEM. arXiv preprint arXiv: 1303.3997doi.org/10.48550/arXiv.1303.3997). SNP variation was detected for each chromosome with the bcftools program (Li H 2011. Bioinformatics 27 (21): 2987-93) and the final VCF file was filtered with vcftools (Danecek P et al., 2011. Bioinformatics 27 (15): 2156-8) using --maf and 0.01 and --minQ 30. code (available at github.com/udiland/agren_seq_snps). Variants within the coding sequence of Lr/Yr548 were extracted from the VCF file of chromosome 6S. Variants and genotypes were filtered out to obtain a matrix with no missing data. The variants were used for maximum likelihood tree construction using the phangorn R package with 100 bootstraps. Root was determined using MAD.R script obtained from: www.mikrobio.uni-kiel.de/de/ag-dagan/ressourcen/mad2-2.zip.

### Structural Analysis and 3D Modeling

The secondary structure of the Lr/Yr 548 protein was predicted using NLRScape (nlrscape.biochim.ro) (Martin E C et al., 2022. Nucleic Acids Res doi: 10.1093/nar/gkac1014), while the number of LRR repeats was analyzed using LRR predictor (Irrpredictor.biochim.ro) (Martin E C et al., 2020. Genes 11(3):286). Finally, the three dimensional structure was predicted using Phyre2 (www.sbg.bio.ic.ac.uk/˜phyre2) (Kelley L A et al., 2015. Nature protocols 10(6):845-58).

Ae. longissima Diversity Panel Phenotyping, Sequencing, and Association Genetics

Genotype by sequencing (GBS) data of the collection (Page R et al., 2023. Frontiers in Plant Science (in review)) and phenotypic disease resistance data (Huang et al. 2018, ibid) were used to select 138 genotypically and phenotypically diverse accessions using Core Hunter R package (De Beukelaer H et al., 2018. BMC bioinformatics 19 (1): 1-2) for the AgRenSeq experiment. Of these accessions, a subset of 36 accessions was selected using the above-mentioned data and methods to be sequenced with 250 bp paired-end (PE) reads instead of 150 bp PE reads. These 36 accessions were used as references in the AgRenSeq analysis. The accessions were phenotyped, as described above and genomic DNA was extracted from plant tissues using the CTAB protocol as described by Millet et al., 2014. In order to filter genes representing the NLR repertoire, the extracted DNA was subjected to NLR capture using a pan-cereal NLR oligo array (Arbor Biosciences, Michigan, USA; Arora et al., 2019. Nature biotechnology 37(2):139-43) and the captured DNA was sequenced on an Illumina HiSeq with 150 bp PE reads (Novogene, China). 36 genotypes were then selected and sequenced with 250 bp PE to be used as references in AgRenSeq. To assemble contigs of these 36 samples BFC (github.com/lh3/bfc) was used for error correction, with k=128 based on kmergenie (kmergenie.bx.psu.edu) estimate and the contigs were assembled using minia (github.com/GATB/minia). Association genetics was then used to identify candidate rust resistance R genes as described in Arora et al., 2019 (ibid).

### Preparation of Recombinant DNA Fragments and Plasmid Constructs

The binary vector pRC3646 (kindly provided by Wisconsin Crop Innovation Center) that contains the bar-selectable marker gene was used as the backbone for plasmid construction.

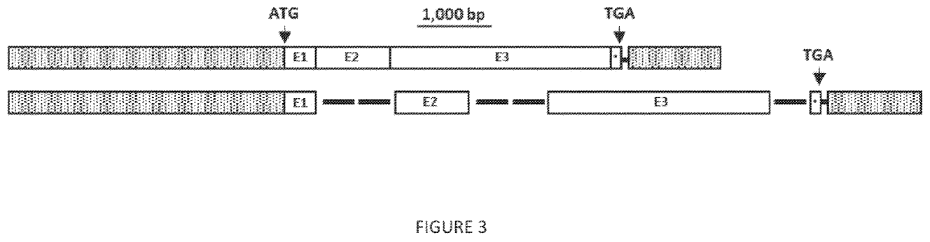

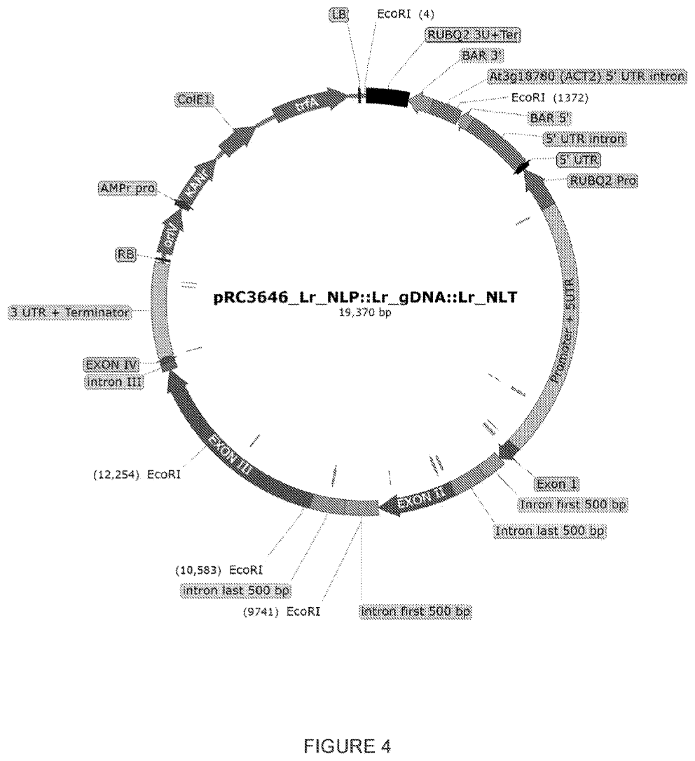

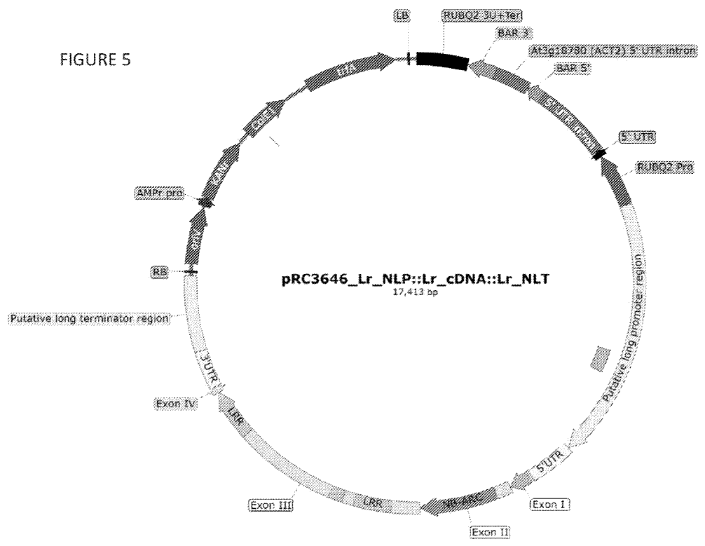

Genomic construct. Two genomic fragments, 3,831 bp upstream of the start codon and 1,431 bp downstream of the stop codon of the Lr/Yr548 resistance gene, were PCR amplified from gDNA of the Ae. longissima resistant accession AEG-6782-2. Due to the large size of introns (>12 kb), for expression of a genomic clone, four fragments that contained no more than 1.0 kb of each intron were generated (FIG. 3). To do so, four different fragments containing the exons and 5′ and 3′ short regions of the respective introns were amplified. These four DNA fragments were ligated and cloned in a Bsal-digested pUDP vector using the NEB® Golden Gate Assembly Kit (BsmBI-v2) and transformed into DH5a competent E. coli cells. PCR amplification of the cloned genomic DNA fragment produced a 6,490 bp DNA fragment containing the four exons, the two shortened intron and the intact third intron (SEQ ID NO:67). Next, the gene, the 5′ UTR and promoter (3,831 bp) and 3′ UTR and terminator (1,413 bp) fragments were combined using Gibson NEBuilder HiFi DNA Assembly Cloning Kit (New England Biolabs), cloned into BsaI-digested pRC3646 vector resulting in pRC3646_Lr_NLP::Lr_gDNA::Lr_NLT vector (FIG. 4) that was transformed into NEB® Stable Competent E. coli cells. The cDNA construct was similarly produced but full-length coding sequence replaced the genomic coding sequence (SEQ ID NO:68) (FIG. 5). Both plasmids were fully sequenced before transfer to Agrobacterium tumefaciens strain AGL-1 for wheat transformation; details of primers (F1-R1-F7-R7) are listed in Table 1.

TABLE 1
Primers used

| Gene | Primer | | | Amplicon |
|---|---|---|---|---|
| name | name | Sequence 5′→3′*^{∧} | Assay type | (bp) |
| Lr/Yr548 | F1 | cccttgacccgaatctcccgcgcgcgTGTCCG | Transgenic | 3891 |
| | R1 | GCCTGCAATTCAC (SEQ ID NO: 18) | construct cloning | |
| | | ACGGCACTCGCGATCATCTCC (SEQ | | |
| | | ID NO: 19) | | |
| Lr/Yr548 | F2 | gcgccgtctcgctcgATGAGCGGGGTTG | Transgenic | 656 |
| | R2 | GGGAG (SEQ ID NO: 20) | construct cloning | |
| | | gcgccgtctcgTGTCAGGCCAGGACCGA | | |
| | | GC (SEQ ID NO: 21) | | |
| Lr/Yr548 | F3 | gcgccgtctcaGACAGTGATTGCTTCGG | Transgenic | 1443 |
| | R3 | CGATAAG (SEQ ID NO: 22) | construct cloning | |
| | | gcgccgtctcgATCTCTCAAACCCAAATC | | |
| | | AACCATCC (SEQ ID NO: 23) | | |
| Lr/Yr548 | F4 | gcgccgtctcgAGATGAAGATCAAAAA | Transgenic | 764 |
| | R4 | GACACATTAGAGTCTCTC (SEQ ID | construct cloning | |
| | | NO: 24) | | |
| | | gcgccgtctcgGAAGCATGGGAAGGCA | | |
| | | ATTCATCG (SEQ ID NO: 25) | | |
| Lr/Yr548 | F5 | gcgccgtctcgcttcTGTGGAAATTAAAT | Transgenic | 3731 |
| | R5 | TATCATATATCCACG (SEQ ID NO: 26) | construct cloning | |
| | | gcgccgtctcgctcgTCAAGAAGTCGATG | | |
| | | GTCCAGC (SEQ ID NO: 27) | | |
| Lr/Yr548 | F6 | ATGAGCGGGGTTGGGGAG (SEQ ID | Transgenic | 6490 |
| | R6 | NO: 28) | construct cloning | |
| | | TCAAGAAGTCGATGGTCCAGC (SEQ | | |
| | | ID NO: 29) | | |
| Lr/Yr548 | F7 | AGAAGCTGGACCATCGACTTCTTG | Transgenic | 1482 |
| | R7 | (SEQ ID NO: 30) | construct cloning | |
| | | tttaaacggatcacttcgtgcgtcgacgGCTG | | |
| | | TGGACTATCTGGAC (SEQ ID NO: 31) | | |
| Bar | F8 | ATGAGCCCAGAACGACG (SEQ ID | Transgenic plant | 995 |
| | R8 | NO: 32) | analysis | |
| | | TCAAATCTCGGTGACGGG (SEQ ID | | |
| | | NO: 33) | | |
| Lr/Yr548 | F9 | CAGCTCATCTCCCTTCAAGAAT (SEQ | Transgenic plant | 1873 |
| | R9 | ID NO: 14) | analysis | |
| | | GTTTACCCGCCAATATATCCTGT | | |
| | | (SEQ ID NO: 15) | | |
| Lr/Yr548 | F10 | TTTAAAGGCGAGGTGGATAAAATGG | Expression analysis | 227 |
| | R10 | (SEQ ID NO: 16) | | |
| | | GCTTGATTGTTCCCCGAAAAGAATAC | | |
| | | C (SEQ ID NO: 17) | | |
| D6PK-like | F11 | GGAAGTATTTCCCCGAACAAGC | Expression analysis | 180 |
| | | (SEQ ID NO: 34) | | |
| | R11 | GGCTCACATCACAACGAAGG (SEQ | | |
| | | ID NO: 35) | | |
| Bar | F12 | TACCGGCAGGCTGAAGTC (SEQ ID | Copy number | 60 |
| | R12 | NO: 36) | analysis | |
| | | TTCAAGCACGGGAACTGG (SEQ ID | | |
| | | NO: 37) | | |
| PINb | F13 | ACCTTCGCGCAATACTCAG (SEQ ID | Copy number | 68 |
| | R13 | NO: 38) | analysis | |
| | | TGTTGAGAACCACCTCCTCC (SEQ ID | | |
| | | NO: 39) | | |
| Bar | Probe | AGCTGCCAGAAACCCACGTCAT (SEQ | Copy number | — |
| | 1 | ID NO: 40) | analysis | |
| PINb | Probe | ACTTCATTGTACCAGCCGCCAACT | Copy number | — |
| | 2 | (SEQ ID NO: 41) | analysis | |

*Lower case letter indicates non-priming nucleotides;
^{∧}Bold letter in primer sequence indicates altered nucleotide for site directed mutagenesis to domesticate BsmBI site.

### Wheat Transformation and Evaluation of Resistance to Rust in Transgenic Wheat Seedlings

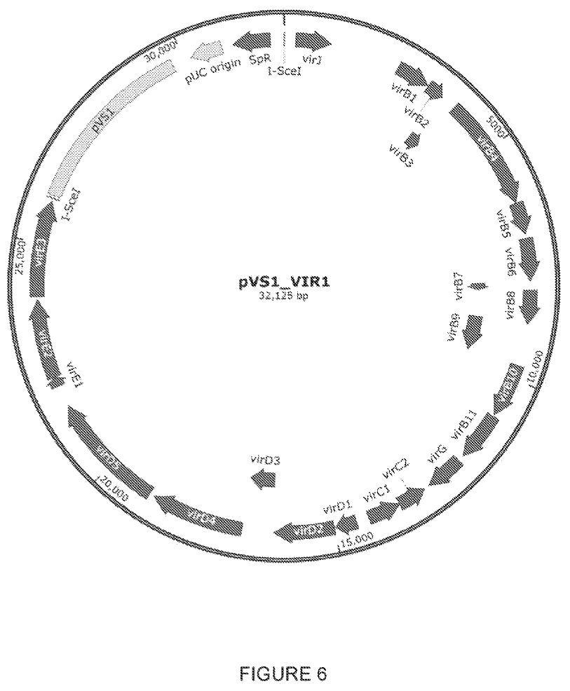

Final confirmation of activity of the cloned gene was performed by transformation into wheat lines according to Hayta et al., 2021 and testing the response of the transformed plants to leaf and stripe rust isolates. The validated binary vectors pRC3646: Lr_cDNA or pRC3646: Lr_gDNA were co-transformed with pVS1-VIR (Zhang R et al., 2019. Nature Plants vol. 5; doi.org/10.1038/s41477-019-0405-0; FIG. 6) into A. tumefaciens strain

AGL-1 using heat shock method. The AGL-1 having both plasmids (binary vector harboring the novel gene and pVS1-VIR helper plasmid) were verified by PCR. The confirmed AGL-1 strain used of hexaploid wheat cv. Fielder (USDA), according to Hayta et al., 2021.

The transformed plants were screened by PCR, using primers against Bar and Lr/Yr548 genes (F8-R8 and F9-R9 respectively, Table 1 hereinabove) RT-PCR analysis, using primers against Lr/Yr548 and D6PK-like genes (F10-R10 [SEQ ID NOs: 16-17] and F11-R11 [SEQ ID NOs: 34-35] respectively, Table 1) and using a Basta (Glufosinate ammonium) leaf spray assay. The T₁ plants expressing the novel rust resistance gene were infected with spores of the leaf rust isolates #12460, #12337 and #526-24 to assess the resistance level as described above in “Plant inoculation and phenotyping”.

### Microscopy

To visualize the average infection area, the infected leaves were detached at 2 and 14 dpi and stained with WGA-FITC (L4895-10 MG; Sigma) as described previously (Zhang W et al., 2017. Proceedings of the national academy of sciences 114 (45): E9483-92) with minor modification. Briefly, the leaves were cut into 2-cm pieces and placed in a 10 ml centrifuge tube with 5 ml of 1 M KOH and 0.05% Silwet L-77. After 12 h, the KOH solution was gently poured off and washed with 10 ml of 50 mM Tris (pH 7.5). This solution was then replaced with another 10 ml of 50 mM Tris (pH 7.5). After 20 min, the Tris solution was removed and replaced with 5 ml of 20 ug/ml WGA-FITC. Tissue was stained for 15 min and then washed with 50 mM Tris (pH 7.5). The WGA-FITC-stained tissue was examined under blue light excitation with an Axio Zoom V.16 (Zeiss).

### Genome Assembly

Ae. longissima libraries were sequenced by Novogene (en.novogene.com) on a PacBio Sequel II (Pacific Biosciences of California, Inc.) using seven SMRT cells (Rhoads A et al., 2015. Genomics, proteomics & bioinformatics. 2015 13(5):278-89; Eid J et al., 2009. Science 323(5910):133-8). A SMRTbell library was generated by fragmentation of genomic DNA to appropriate sizes. Then DNA fragments were damage-repaired, end-repaired, and A-tailed. The SMRTbell library was produced by ligating universal hairpin adapters onto double-stranded DNA fragments. After the exonuclease and AMPure PB beads purification steps, sequencing primer was annealed to the SMRTbell templates, followed by binding of the sequencing polymerase to the annealed templates. The software SMRTlink was used to filter and process original sequencing results, with minLength 0, minReadScore 0.8 as parameters. The HiFi read assembly was done using hifiasm (github.com/chhylp123/hifiasm; version 0.14) with default parameters. The pseudomolecules were constructed using the TRITEX pipeline as described in Marone et al., 2022.

### Example 1

### Identification of Ae. longissima Rust-Resistance Conferring Gene

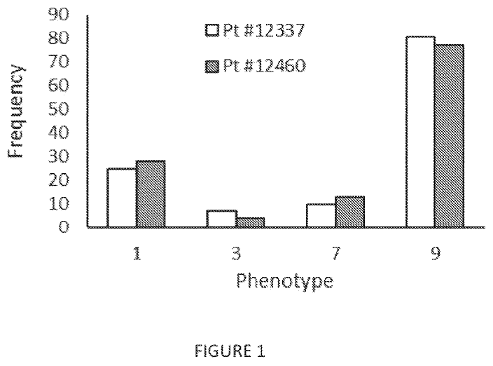

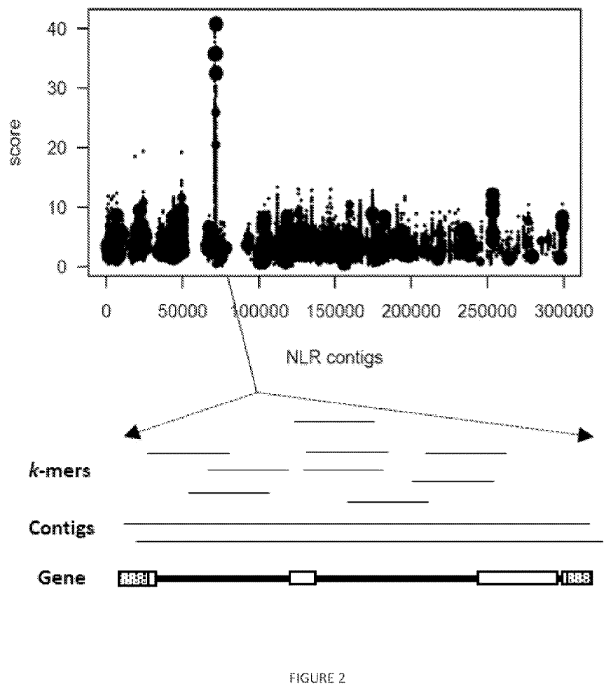

The diversity panel of Ae. longissima accessions was phenotyped at seedling stage as described in the “Material and Methods” hereinabove using the two Pt isolates, #12460 (race MCDTB) and #12337 (race MFPPB). Both isolates showed similar results, with resistance frequency of 30% (FIG. 1). To identify candidate Lr resistance genes the AgRenSeq pipeline using the pan cereal NLR capture library (Arora S et al., 2019, ibid; Steuernagel et al., 2016. Nature Biotech 34:652-655) and the phenotyping results of the two Pt isolates were followed (FIG. 2) de novo assemblies from the Illumina short-read sequences were generated and contigs were assembled with the aid of the Ae. longissima reference genome (Avni et al., 2022, ibid). Thirty-six accessions were sequenced with Illumine 250PE and the rest with 150PE. The 36 accessions were used as reference in the AgRenSeq pipeline and contain 119628 to 426762 (average 296201) NLR contigs per accession. Using NLR-Parser (Steuernagel et al., 2016, ibid) the NLR repertoire for each contig assembly was obtained based on unique motifs.

Next, k-mer-based AgRenSeq analysis was carried out to identify Lr resistance genes in the phenotyped panel and the k-mers were projected directly onto NLR assemblies generated from read data of resistant accessions to obtain long contigs including full-length NLRs. To identify putative Lr resistant genes, an Ae. longissima high quality reference genomic sequence was produced from Ae. longissima accession AEG-6782-2 (Avni et al., 2022, ibid), which is resistant to the two Lr isolates, and k-mers associated with #12460 and #12337 resistance were mapped onto the genomic assembly.

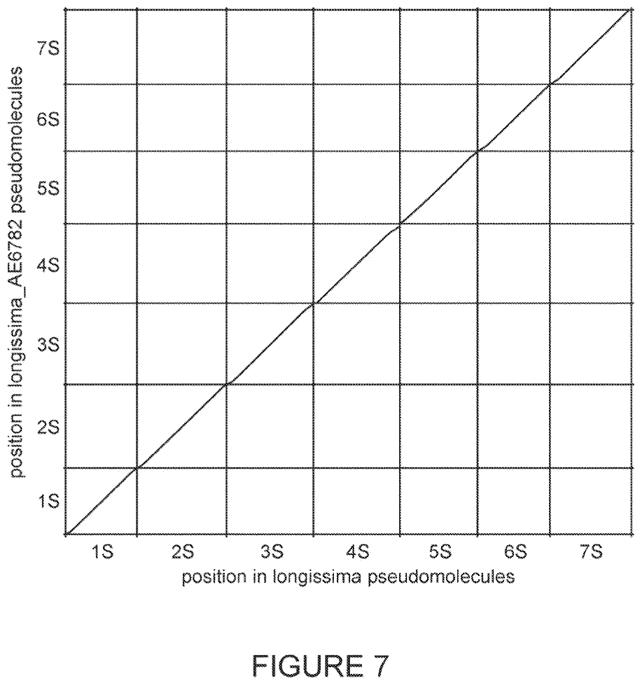

A significant association between both isolates and two contigs, 71128 (SEQ ID NO: 65 and 71798 (SEQ ID NO:66) that were located to an unmapped region in the reference genome (“chromosome unknown”) was found. To precisely map the gene, additional genomic sequence data of the Ae. longissima AEG-6782-2 reference line was generate using PacBio, and an improved genome assembly was we generate (Rhoads et al., 2015, ibid; Sedlazeck F J et al., 2018. Nature Reviews Genetics 19 (6): 329-46). The length of the new HiFi assembly was 6.033 Gb, the N50 increased from 3.8 Mb in the first assembly (Avni et al., 2022, ibid) to 16 Mb (Table 2), and the number of scaffolds decreased accordingly from 130,347 to 2,608, with and average number of 97.8 scaffolds per chromosome. The estimated genome size increased slightly from 5.919 Gb to 5.947 Gb, and the number of unanchored contigs was reduced by more than three orders of magnitude, from 1,854,921 in the first assembly to 1,238 in the new assembly (FIG. 7). The improved assembly enabled mapping the two AgRenSeq LR candidate contigs to an ORF on chromosome 6S at position 51,756,423-51,772,629 (16,207 bp, SEQ ID NO:6).

TABLE 2
Comparison of Ae. longissima genome assembly parameters between
the previous (Avni et al., 2022, ibid) and the current versions

| | Ae. longissima v1.0 (Avni et al., 2022, ibid) | | | | | Ae. longissima v2.0 | | | | |
|---|---|---|---|---|---|---|---|---|---|---|
| | | | | max | min | | | | max | min |
| | length | | N50 | length | length | length | | N50 | length | length |
| chr | (Mb) | Ncontig | (Mb) | (Mb) | (kb) | (Mb) | Ncontig | (Mb) | (Mb) | (kb) |
| chr1S | 742.3 | 289 | 4.5 | 18.7 | 300.8 | 748.6 | 90 | 15.9 | 40.5 | 525.3 |
| chr2S | 956.2 | 362 | 4.8 | 20.8 | 302.1 | 949.3 | 95 | 16.5 | 56.4 | 567.6 |
| chr3S | 916.7 | 368 | 4.8 | 24.8 | 304.3 | 915.3 | 106 | 18.3 | 46.4 | 521.2 |
| chr4S | 874.5 | 346 | 4.7 | 20.8 | 300.5 | 901.7 | 116 | 14.7 | 63.8 | 675.3 |
| chr5S | 816.3 | 347 | 4.3 | 16.5 | 301 | 822.1 | 94 | 15.1 | 35.2 | 643.5 |
| chr6S | 750.2 | 312 | 3.9 | 17.4 | 320.9 | 755.6 | 84 | 17.2 | 48.3 | 577.8 |
| chr7S | 862.6 | 339 | 4.3 | 17.1 | 302.7 | 854.1 | 100 | 19.2 | 68.2 | 521.1 |
| 1S-7S | 5,918.8 | 2,363 | 4.5 | 24.8 | 300.5 | 5,946.7 | 685 | 16 | 68.2 | 521.1 |
| Un | 1,710.2 | 1,854,921 | 0 | 0.3 | 0.2 | 86.1 | 1,238 | 0.1 | 0.8 | 16.6 |
| Total | 7,629.0 | | | | | 6,032.8 | | | | |

### Example 2

### Identification of Domains Within the Resistance-Conferring Gene

A polypeptide domain refers to a set of conserved amino acids located at specific positions along an alignment of sequences of evolutionarily related proteins. While amino acids at other positions can vary between homologues, amino acids that are highly conserved, and particularly amino acids that are highly conserved at specific positions indicate amino acids that are likely essential in the structure, stability and/or function of a protein. Identified by their high degree of conservation in aligned sequences of a family of protein homologues, they can be used as identifiers to determine if any polypeptide in question belongs to a previously identified polypeptide family.

Table 3 below summarizes known domains that have been identified within the resistance-conferring polynucleotides of the invention.

TABLE 3
Domains included in the resistance-conferring gene

| Domain | Location | Description |
|---|---|---|
| Rx_N | 34-252 | The RxN domain is present in the N-termini |
| domain | | of a major type of NLR proteins, termed |
| | | coiled-coil domain |
| | | Hao Wet al., 2013. Structural basis for the |
| | | interaction between the potato virus × |
| | | resistance protein (Rx) and its cofactor |
| | | Ran GTPase-activating protein 2 (RanGAP2). |
| | | J. Biol. Chem. 288, 35868-35876. |
| | | doi: 10.1074/jbc.m113.517417 |
| NB-ARC | 5523-6290 | a novel signaling motif shared by plant |
| Domain | | resistance gene products and regulators |
| | | of cell death in animals |
| | | Van der Biezen E A and Jones J D. |
| | | 1998. Curr Biol 26; 8(7): R226-7. |
| | | doi: 10.1016/s0960-9822(98)70145-9. |
| LRR | 13397-15937 | leucine-rich repeat domain is a common |
| Domains | | motif found in more than 2,000 proteins, from |
| | | viruses to eukaryotes, and it is involved in |
| | | protein-protein interactions and ligand binding |
| | | Jones D A, Jones J D G: The role of leucine- |
| | | rich repeat proteins in plant defenses. |
| | | Adv Bot Res. 1997, 24: 90-167. |

### Example 3

### Producing Wheat Plants Having Enhanced Resistance to Leaf Rust and Stripe Rust Disease

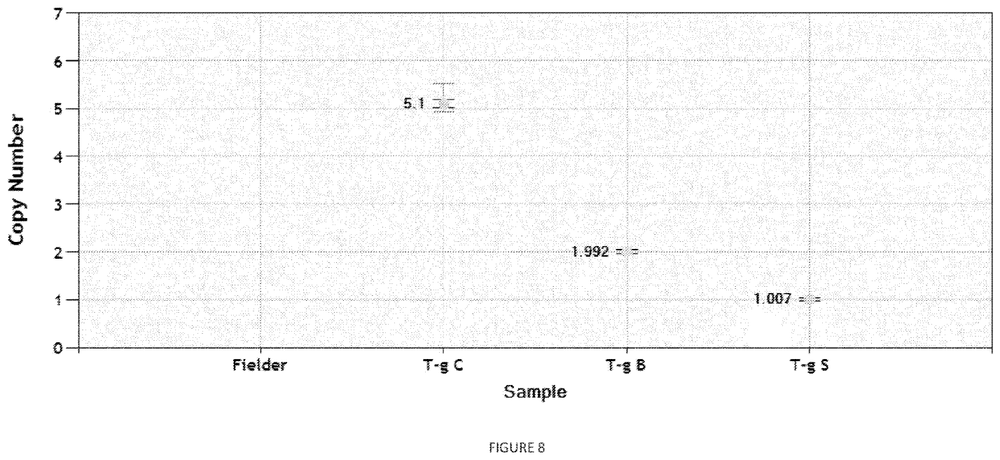

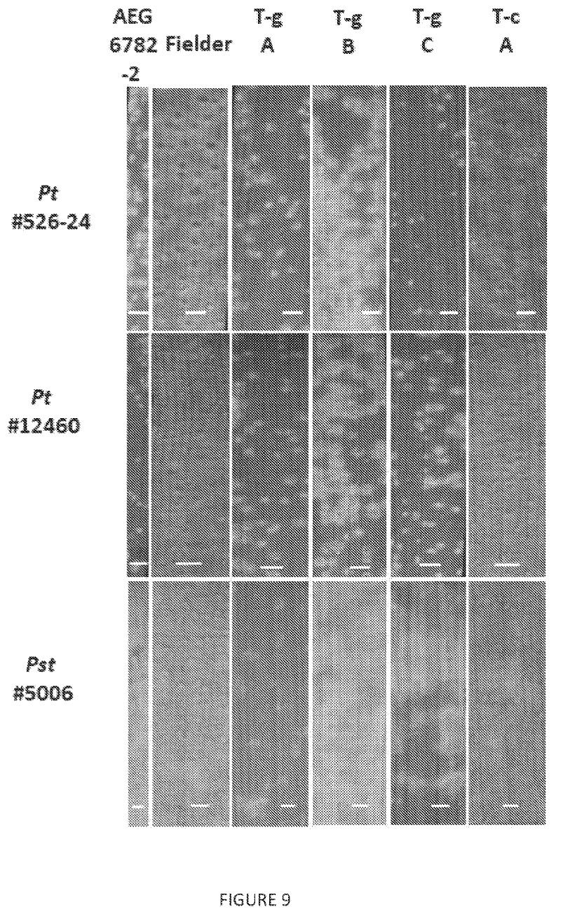

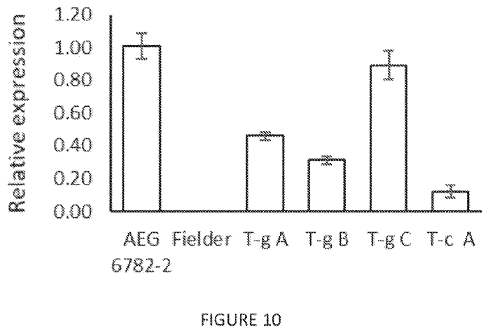

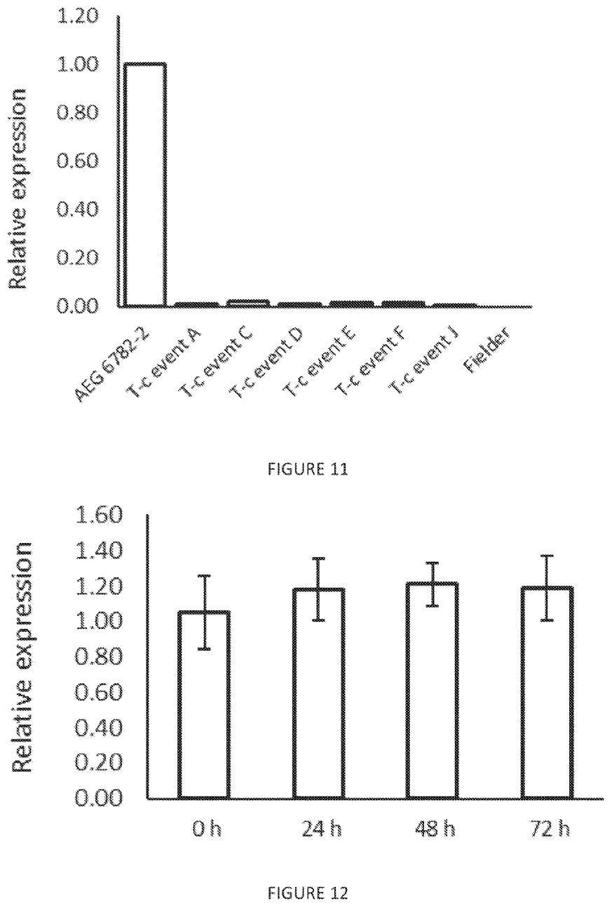

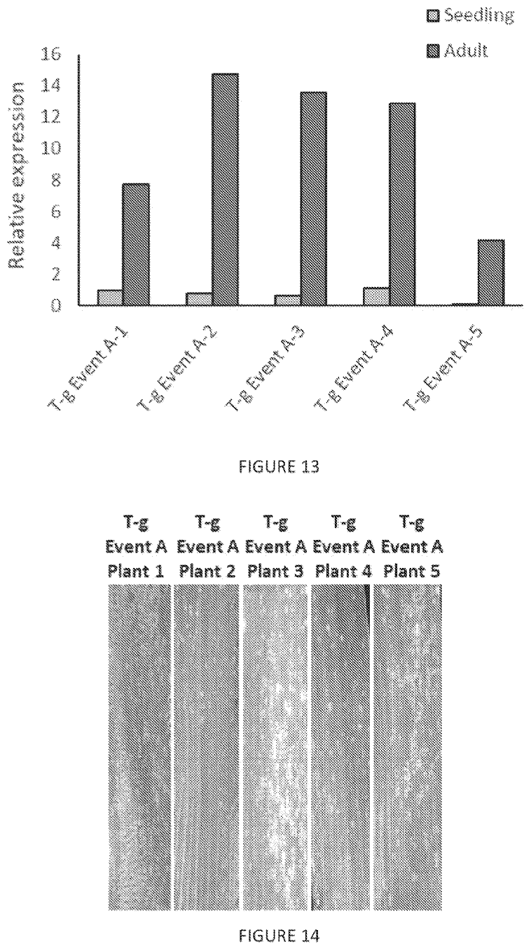

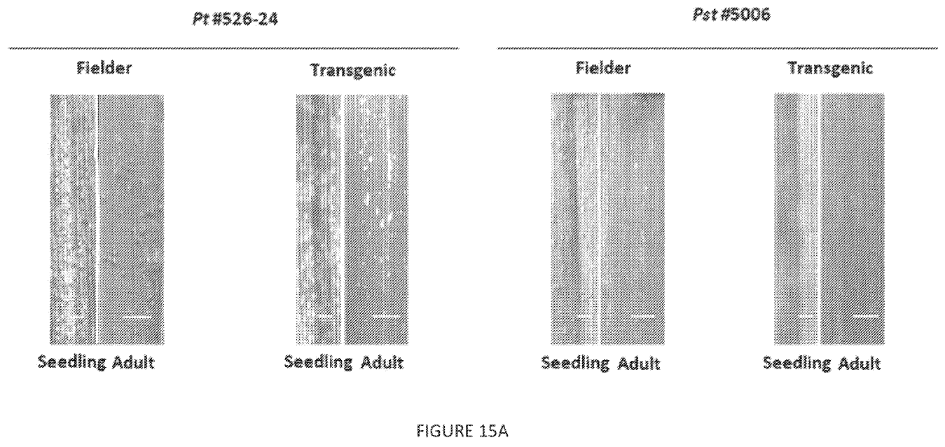

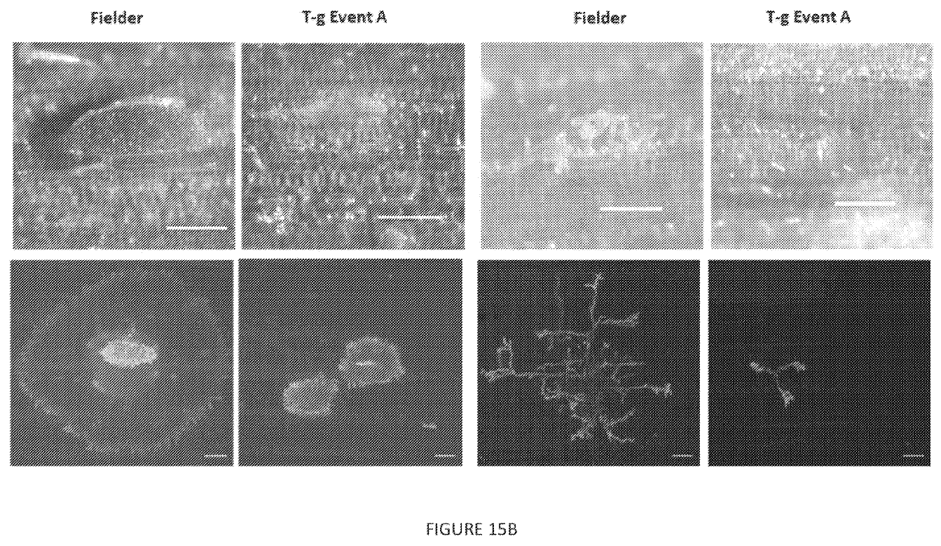

To verify the function of the Ae. longissima gene in resistance to Pt and Pst, transgenic wheat cv. Fielder were produced, expressing either the cDNA (SEQ ID NO:3) or a synthetic genomic clone that included the four exons separated by shortened introns, both with the native promoter (SEQ ID NO:4) and termination sequences (SEQ ID NO:5), see methods and FIG. 3 for details). Independent primary transgenic events (T₀) were generated, from each, three events were selected with various copy numbers of either the cDNA or the synthetic genomic clones (FIG. 8). Homozygous T1 and T2 progenies were produced, and their response to infection with the Pt and Pst isolates was tested. The three cDNA transgenic lines were all sensitive to the Pt #526-24 and #12460 and Pst #5006 isolates, whereas the three gDNA transgenic lines showed variable degrees of resistance (FIG. 9). qPCR analysis revealed that the Lr/Yr548 gene expression levels in the cDNA transgenic plants were at least 2.5-fold lower than expression levels in the gDNA plants, and approximately six-fold lower than in Ae. longissima (FIG. 10). The relatively low expression levels in the several cDNA transgenic lines tested (FIG. 11) suggested that the introns within the coding sequence as well as the 3′ UTR are important for proper Lr/Yr548 gene expression. Further analysis revealed that expression of the Lr/Yr548 gene did not differ between infected and uninfected plants (FIG. 12), while it increased with plant age (FIG. 13) and correlated with the level of resistance (FIG. 14). These results confirmed the function of the Ae. longissima Lr/Yr548 gene in resistance to both Pt and Pst and highlighted the importance of the introns for proper gene expression.

### Example 4

### The Lr/Yr548 Gene Conferes Quantitative Resistance in Seedlings and Adult Plants

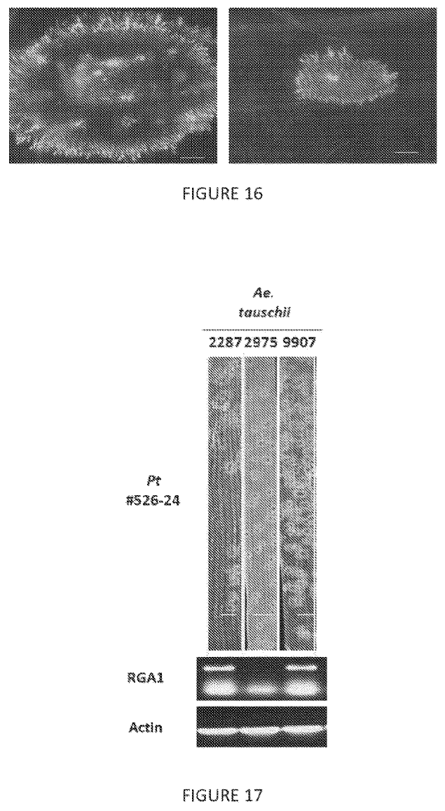

To gain insight into the mode of resistance conferred by the Lr/Yr548 protein, wheat cv. Fielder and derived resistant transgenic lines were inoculated with Pt and Pst isolates, and then the leaf samples were stained with FITC-labelled wheat germ agglutinin (WGA), which specifically stains fungal cell walls (Meyberg M. 1988. Histochemistry 88(2):197-9). Both the Pt and Pst isolates were able to penetrate and grow within leaf tissues of the resistant plants, but fungal development was much slower, and the area occupied by the fungi was significantly smaller than in the susceptible wheat cv. Fielder (FIG. 15A-B).

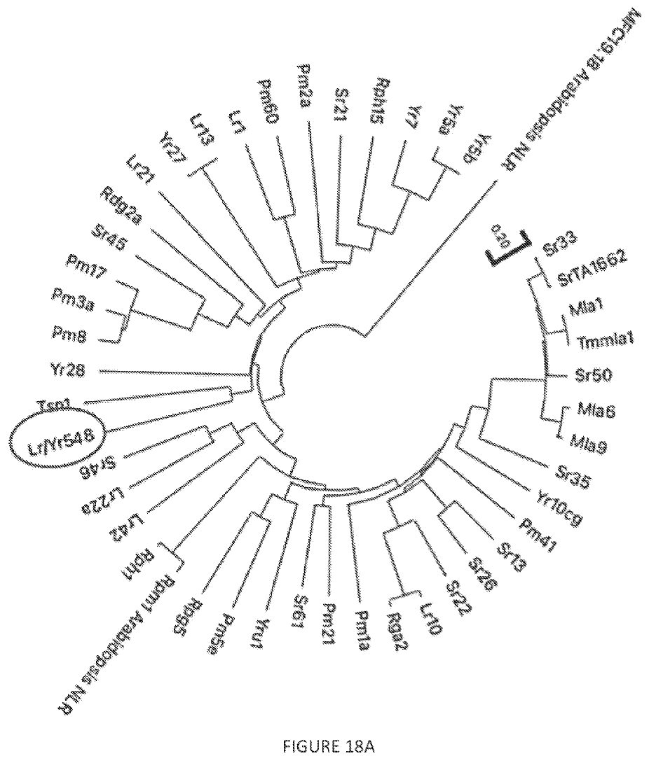

Similar results were observed on susceptible and resistant Ae. longissima plants (FIG. 16), and while Lr/Yr548 gene expression increased with plant age (FIG. 13), there was no difference in the disease response between seedling and adult (flag leaf stage) plants (FIG. 15A). Additionally, the transgenic plants were found susceptible to stem rust (Pgt) and powdery mildew (Blumeria graminis) (data not shown). Without wishing to be bound by any specific theory or mechanism of action, the presence of the Lr/Yr548 gene slows down fungal infection, but it does not lead to induction of a hyper-sensitive response (HR). Nevertheless, and while the expressed gene does not completely stop fungal progression, it prevents the development of the two diseases in seedlings and mature plants. The resistance conferred by the Lr/Yr548 is specific to Pt causing leaf rust and to Pst causing stripe rust, but is ineffective against other biotrophic wheat pathogens.

Selection of transformed plants comprising the resistance-conferring polynucleotides of the invention is further performed by amplifying at least one marker segment using the primers listed in Table 4 hereinbelow.

TABLE 4
Primer pairs amplifying marker segments of the resistance
conferring gene of the invention

| Name | Sequence 5′→3′ | SEQ ID NO. |
|---|---|---|
| Lr_Fw14 | TAATCAAACTCCTCGAAGATGAGACCACTA | 42 |
| Lr_Rv14 | CCAGCAGGTCCTCAACGTCGTGGGAGATG | 43 |
| Lr_Fw15 | GAGATAAGCTGGACAAGCTGAAGGAC | 44 |
| Lr_Rv15 | CTGCCATGCTGCGATGGACTTATTCC | 45 |
| Lr_Fw16 | AAAATTAATAAATTTGATATTGCGAAGCC | 46 |
| Lr_Rv16 | CTTTGATGTCGAAGATGTTGAATTTCTCA | 47 |
| Lr_Fw17 | TGGTTGATTTGGGTTTGAGAGACGAAGA | 48 |
| Lr_Rv17 | ACATCCCAAGAAGGTAGTCGATGCAGT | 49 |
| Lr_Fw18 | GGATTATAATTACTACACGTAACCAAAGGA | 50 |
| Lr_Rv18 | CTCTAATGTGTCTTTTTGATCTTCGTC | 51 |
| Lr_Fw19 | TTTCCTCAACTTATCAGGGTGTTCCGTCC | 52 |
| Lr_Rv19 | TTTTGAGCAACCGGACAAATTTAGGAATGA | 53 |
| Lr_Fw20 | TCCTGATAAGTTTGGTAGCCTACCAAATATCTC | 54 |
| Lr_Rv20 | GGTATTTCAAATGAATAAGCTGGCAAAATGATTCC | 55 |
| Lr_Fw21 | TTGCCATGGCCTCAGGGAACTCCCAGAG | 56 |
| Lr_Rv21 | AATTGAGATGCCTCAGCTTAAACAAGCTA | 57 |
| Lr_Fw22 | TTGTTAACCTTGAGTCTTTGGAGCATTTCG | 58 |
| Lr_Rv22 | TGCAGCTTGGAACAACTTATTAGGTTCAAT | 59 |
| Lr_Fw23 | AGTTGCCACGAGCTAAAAGACCTC | 60 |
| Lr_Rv23 | GAGGATGGGAGCTTTTCAAGCCTCATACAG | 61 |

### Example 5

### Phylogenetic Analysis

A global BLAST search of the NCBI and cereals-specific databases using Lr/Yr548 coding sequence (CDS) as a query revealed genes with significant homology in plant species from three tribes: Triticeae, Poeae and Brachypodieae (data not shown). The two closest homologues were the Ae. tauschii RGA1 putative disease resistance gene, which is 87% identical to the predicted Lr/Yr548 CDS (SEQ ID NO:3), and Triticum urartu RGA2-like putative disease resistance gene, with 83% identity (Tables 5 and 6). All other 10 predicted genes shared less than 80% homology or had less than 80% coverage. Interestingly, global BLAST search using the Lr/Yr548 protein sequence as a query, showed a lower level of homology to the Ae. tauschii RGA1 putative disease resistance gene (79%) and the rest of the sequences had less than 65% homology (data not shown).

TABLE 5
Homology level to the Lr/Yr548 CDS sequence in other species

| | | | Query | E | Per. | Acc. | |
|---|---|---|---|---|---|---|---|
| Description | Tribe | Scientific Name | Cover | value | ident | Len | Accession |
| PREDICTED: Aegilops tauschii subsp. | Triticeae | Aegilops tauschii | 97% | 0 | 86.56 | 5391 | XM_020307273.3 |
| strangulata putative disease resistance | | subsp. strangulata | | | | | |
| protein RGA1 (LOC109748238), | | | | | | | |
| transcript variant X1, mRNA | | | | | | | |
| PREDICTED: Triticum urartu | Triticeae | Triticum urartu | 86% | 0 | 82.98 | 5857 | XM_048710018.1 |
| disease resistance protein | | | | | | | |
| RGA2-like (LOC125545958), | | | | | | | |
| transcript variant X1, mRNA | | | | | | | |
| PREDICTED: Triticum aestivum | Triticeae | Triticum aestivum | 80% | 0 | 73.35 | 5500 | XM_044535828.1 |
| putative disease resistance protein | | | | | | | |
| RGA3 (LOC123114383), mRNA | | | | | | | |
| PREDICTED: Triticum dicoccoides | Triticeae | Triticum | 80% | 0 | 73.35 | 5119 | XM_037588700.1 |
| putative disease resistance protein | | dicoccoides | | | | | |
| RGA3 (LOC119312915), mRNA | | | | | | | |
| PREDICTED: Triticum urartu | Triticeae | Triticum urartu | 74% | 0 | 77.59 | 6258 | XM_048710033.1 |
| disease resistance protein | | | | | | | |
| RGA2-like (LOC125545966), | | | | | | | |
| transcript variant X1, mRNA | | | | | | | |
| PREDICTED: Triticum aestivum | Triticeae | Triticum aestivum | 62% | 0 | 74.38 | 4184 | XM_044535983.1 |
| disease resistance protein | | | | | | | |
| RGA2-like (LOC123114490), mRNA | | | | | | | |
| Dasypyrum villosum isolate | Triticeae | Dasypyrum | 60% | 0 | 81.94 | 3810 | MZ672775.1 |
| Hv_Contig_894_nlr_1 NBS-LRR | | villosum | | | | | |
| disease resistance protein mRNA, | | | | | | | |
| partial cds | | | | | | | |
| PREDICTED: Triticum aestivum | Triticeae | Triticum aestivum | 60% | 0 | 77.81 | 4984 | XM_044498128.1 |
| putative disease resistance protein | | | | | | | |
| RGA4 (LOC123075549), mRNA | | | | | | | |
| PREDICTED: Triticum urartu putative | Triticeae | Triticum urartu | 60% | 0 | 73.2 | 4673 | XM_048687581.1 |
| disease resistance protein RGA3 | | | | | | | |
| (LOC125522530), mRNA | | | | | | | |
| PREDICTED: Lolium rigidum disease | Poeae | Lolium rigidum | 56% | 0 | 79.13 | 4662 | XM_047202197.1 |
| resistance protein RGA2-like | | | | | | | |
| (LOC124664751), mRNA | | | | | | | |
| PREDICTED: Lolium rigidum | Poeae | Lolium rigidum | 55% | 0 | 78.87 | 4200 | XM_047202225.1 |
| putative disease resistance protein | | | | | | | |
| RGA4 (LOC124664787), mRNA | | | | | | | |
| PREDICTED: Triticum aestivum | Triticeae | Triticum aestivum | 49% | 0 | 78.91 | 4394 | XM_044503523.1 |
| disease resistance protein | | | | | | | |
| RGA2-like (LOC123080588), | | | | | | | |
| transcript variant X1, mRNA | | | | | | | |
| PREDICTED: Triticum urartu | Triticeae | Triticum urartu | 49% | 0 | 78.86 | 5648 | XM_048710039.1 |
| disease resistance protein | | | | | | | |
| RGA2-like (LOC125545970), | | | | | | | |
| transcript variant X1, mRNA | | | | | | | |
| PREDICTED: Aegilops tauschii subsp. | Triticeae | Aegilops tauschii | 48% | 0 | 83.88 | 3370 | XM_040402871.1 |
| strangulata disease resistance | | subsp. strangulata | | | | | |
| protein RGA2 (LOC109741696), mRNA | | | | | | | |
| PREDICTED: Hordeum vulgare subsp. | Triticeae | Hordeum vulgare | 47% | 0 | 73.82 | 5279 | XM_045118224.1 |
| vulgare putative disease resistance protein | | subsp. vulgare | | | | | |
| RGA3 (LOC123442136), mRNA | | | | | | | |
| PREDICTED: Brachypodium | Brachypodieae | Brachypodium | 46% | 0 | 78.89 | 7247 | XM_024458994.1 |
| distachyon putative disease resistance | | distachyon | | | | | |
| protein RGA3 (LOC100836635), | | | | | | | |
| mRNA | | | | | | | |
| PREDICTED: Triticum aestivum | Triticeae | Triticum aestivum | 46% | 0 | 74.67 | 3958 | XM_044538564.1 |
| disease resistance protein | | | | | | | |
| RGA2-like (LOC123117898), mRNA | | | | | | | |
| PREDICTED: Lolium rigidum putative | Poeae | Lolium rigidum | 45% | 0 | 79.54 | 4077 | XM_047195428.1 |
| disease resistance protein RGA3 | | | | | | | |
| (LOC124656732), mRNA | | | | | | | |
| Dasypyrum villosum isolate | Triticeae | Dasypyrum | 44% | 3.00E−123 | 73.92 | 3567 | MZ672909.1 |
| Hv_Contig_1228_nlr_1 NBS-LRR | | villosum | | | | | |
| disease resistance protein mRNA, | | | | | | | |
| complete cds | | | | | | | |
| PREDICTED: Triticum aestivum | Triticeae | Triticum aestivum | 40% | 0 | 74.25 | 3882 | XM_044544679.1 |
| putative disease resistance protein | | | | | | | |
| RGA3 (LOC123123989), mRNA | | | | | | | |
| PREDICTED: Aegilops tauschii subsp. | Triticeae | Aegilops tauschii | 39% | 0 | 79.31 | 4676 | XM_045233877.1 |
| strangulata disease resistance protein | | subsp. strangulata | | | | | |
| RGA2-like (LOC109741700), mRNA | | | | | | | |
| PREDICTED: Aegilops tauschii subsp. | Triticeae | Aegilops tauschii | 37% | 1.00E−166 | 74.69 | 4724 | XM_020322770.3 |
| strangulata disease resistance protein | | subsp. strangulata | | | | | |
| RGA2 (LOC109763900), transcript | | | | | | | |
| variant X1, mRNA | | | | | | | |
| PREDICTED: Triticum aestivum | Triticeae | Triticum aestivum | 37% | 1.00E−166 | 74.69 | 4513 | XM_044595780.1 |
| disease resistance protein | | | | | | | |
| RGA2-like (LOC123183052), | | | | | | | |
| transcript variant X1, mRNA | | | | | | | |
| PREDICTED: Aegilops tauschii subsp. | Triticeae | Aegilops tauschii | 21% | 0 | 81.99 | 1587 | XM_040385781.2 |
| strangulata disease resistance protein | | subsp. strangulata | | | | | |
| RGA2 (LOC120961907), transcript | | | | | | | |
| variant X2, mRNA | | | | | | | |
| PREDICTED: Aegilops tauschii subsp. | Triticeae | Aegilops tauschii | 17% | 0 | 87.87 | 5336 | XM_045233875.1 |
| strangulata putative disease resistance | | subsp. strangulata | | | | | |
| protein At3g14460 (LOC109741703), | | | | | | | |
| transcript variant X1, mRNA | | | | | | | |

TABLE 6
Homology level to the Lr/Yr548 protein sequence in other species

| | | Query | E | Per. | Acc. | |
|---|---|---|---|---|---|---|
| Description | Scientific Name | Cover | value | ident | Len | Accession |
| putative disease resistance protein RGA1 | Aegilops tauschii | 97% | 0 | 78.67 | 1346 | XP_020162862.1 |
| [Aegilops tauschii subsp. strangulata] | subsp. strangulata | | | | | |
| disease resistance protein RGA2-like | Triticum aestivum | 97% | 0 | 60.69 | 1506 | XP_044359446.1 |
| [Triticum aestivum] | | | | | | |
| putative disease resistance protein RGA3 | Triticum aestivum | 97% | 0 | 57.82 | 1553 | XP_044359457.1 |
| isoform X2 [Triticum aestivum] | | | | | | |
| putative disease resistance protein RGA3 | Triticum aestivum | 97% | 0 | 57.63 | 1558 | XP_044359453.1 |
| isoform X1 [Triticum aestivum] | | | | | | |
| putative disease resistance protein RGA1 | Aegilops tauschii | 97% | 0 | 58.56 | 1458 | XP_020156333.1 |
| isoform X1 [Aegilops tauschii subsp. | subsp. strangulata | | | | | |
| strangulata] | | | | | | |
| hypothetical protein CFC21_013227 | Triticum aestivum | 97% | 0 | 59.68 | 1540 | KAF6996956.1 |
| [Triticum aestivum] | | | | | | |
| putative disease resistance protein RGA1 | Triticum aestivum | 97% | 0 | 59.68 | 1703 | XP_044451282.1 |
| [Triticum aestivum] | | | | | | |
| putative disease resistance protein RGA1 | Aegilops tauschii | 97% | 0 | 59.54 | 1540 | XP_040245775.1 |
| [Aegilops tauschii subsp. strangulata] | subsp. strangulata | | | | | |
| LOW QUALITY PROTEIN: putative | Brachypodium | 99% | 0 | 61.31 | 1501 | XP_024314765.1 |
| disease resistance protein RGA3 | distachyon | | | | | |
| [Brachypodium distachyon] | | | | | | |
| LOW QUALITY PROTEIN: putative | Brachypodium | 95% | 0 | 63.19 | 1326 | XP_024314762.1 |
| disease resistance protein RGA3 | distachyon | | | | | |
| [Brachypodium distachyon] | | | | | | |
| disease resistance protein RGA2-like | Triticum urartu | 98% | 0 | 61.56 | 1431 | XP_048565996.1 |
| [Triticum urartu] | | | | | | |
| disease resistance protein RGA2-like | Triticum aestivum | 98% | 0 | 61.56 | 1433 | XP_044359458.1 |
| isoform X1 [Triticum aestivum] | | | | | | |
| putative disease resistance protein | Triticum urartu | 97% | 0 | 80.2 | 1407 | XP_048566001.1 |
| RGA1 [Triticum urartu] | | | | | | |
| hypothetical protein CFC21_049919 | Triticum aestivum | 98% | 0 | 61.56 | 1480 | KAF7039982.1 |
| [Triticum aestivum] | | | | | | |
| disease resistance protein RGA2-like | Triticum urartu | 96% | 0 | 63.12 | 1373 | XP_048565990.1 |
| [Triticum urartu] | | | | | | |
| putative disease resistance protein RGA1 | Aegilops tauschii | 97% | 0 | 57.96 | 1779 | XP_020199709.2 |
| [Aegilops tauschii subsp. strangulata] | subsp. strangulata | | | | | |
| disease resistance protein RGA2-like | Triticum aestivum | 98% | 0 | 61.56 | 1431 | XP_044359459.1 |
| isoform X2 [Triticum aestivum] | | | | | | |
| putative disease resistance protein | Lolium rigidum | 97% | 0 | 61.25 | 1358 | XP_047051384.1 |
| RGA3 [Lolium rigidum] | | | | | | |
| putative disease resistance protein | Triticum aestivum | 97% | 0 | 56.76 | 1529 | XP_044391916.1 |
| RGA3 [Triticum aestivum] | | | | | | |
| hypothetical protein BRADI_2g60230v3 | Brachypodium | 98% | 0 | 62.71 | 1405 | PNT73551.1 |
| [Brachypodium distachyon] | distachyon | | | | | |
| putative disease resistance protein RGA3 | Brachypodium | 98% | 0 | 62.71 | 1374 | XP_010232791.2 |
| isoform X1 [Brachypodium distachyon] | distachyon | | | | | |
| hypothetical protein BRADI_2g60230v3 | Brachypodium | 97% | 0 | 62.71 | 1402 | PNT73547.1 |
| [Brachypodium distachyon] | distachyon | | | | | |
| putative disease resistance protein RGA3 | Brachypodium | 97% | 0 | 62.71 | 1371 | XP_014754561.1 |
| isoform X2 [Brachypodium distachyon] | distachyon | | | | | |
| unnamed protein product | Triticum turgidum | 97% | 0 | 57.05 | 1433 | VAI40400.1 |
| [Triticum turgidum subsp. durum] | subsp. durum | | | | | |
| putative disease resistance protein | Triticum | 97% | 0 | 56.85 | 1433 | XP_037444597.1 |
| RGA3 [Triticum dicoccoides] | dicoccoides | | | | | |
| disease resistance protein RGA2-like | Lolium rigidum | 98% | 0 | 61.71 | 1553 | XP_047058153.1 |
| [Lolium rigidum] | | | | | | |
| disease resistance protein RGA2-like | Aegilops tauschii | 98% | 0 | 60.33 | 1423 | XP_045089812.1 |
| [Aegilops tauschii subsp. strangulata] | subsp. strangulata | | | | | |
| putative disease resistance protein | Lolium rigidum | 97% | 0 | 61.06 | 1399 | XP_047058181.1 |
| RGA4 [Lolium rigidum] | | | | | | |
| hypothetical protein BRADI_2g60260v3 | Brachypodium | 97% | 0 | 61.82 | 1353 | KQK11445.1 |
| [Brachypodium distachyon] | distachyon | | | | | |
| putative disease resistance protein RGA3 | Aegilops tauschii | 91% | 0 | 59.4 | 1357 | XP_020156337.1 |
| isoform X2 [Aegilops tauschii | subsp. strangulata | | | | | |
| subsp. strangulata] | | | | | | |
| putative disease resistance protein RGA1 | Triticum | 97% | 0 | 56.11 | 1433 | XP_037444588.1 |
| [Triticum dicoccoides] | dicoccoides | | | | | |
| putative disease resistance protein RGA3 | Aegilops tauschii | 90% | 0 | 59.54 | 1336 | XP_020156338.1 |
| isoform X3 [Aegilops tauschii | subsp. strangulata | | | | | |
| subsp. strangulata] | | | | | | |
| putative disease resistance protein RGA4 | Oryza glaberrima | 98% | 0 | 55.33 | 1760 | XP_052140172.1 |
| [Oryza glaberrima] | | | | | | |
| hypothetical protein BRADI_2g60230v3 | Brachypodium | 97% | 0 | 60.46 | 1361 | PNT73549.1 |
| [Brachypodium distachyon] | distachyon | | | | | |
| Putative disease resistance protein RGA3 | Triticum urartu | 89% | 0 | 63.41 | 1299 | EMS55639.1 |
| [Triticum urartu] | | | | | | |

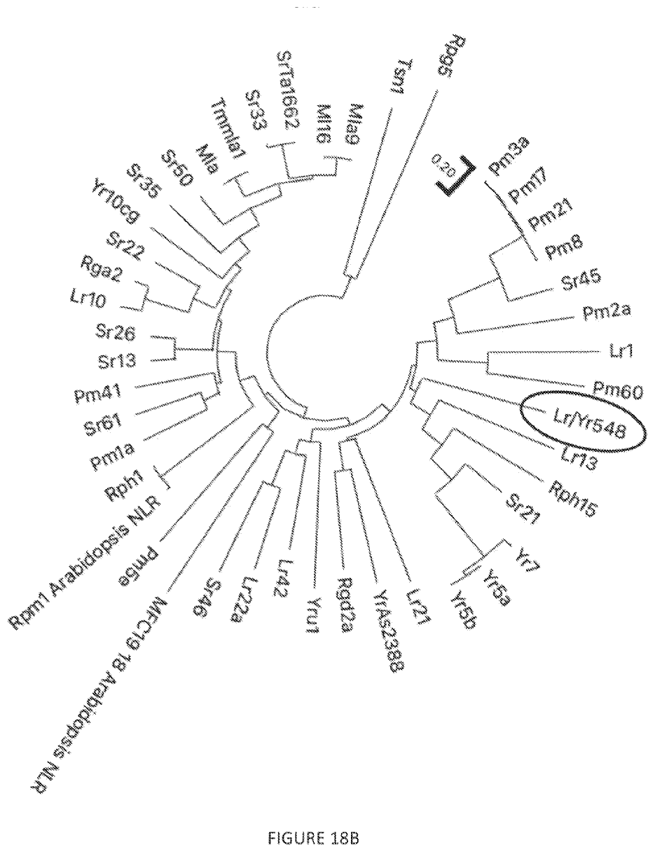

To check if the Ae. tauschii RGA1 is a functional homologue of the Lr/Yr548 gene, the response to Pt #526-24 of three Ae. tauschii accessions, two that harbor the RGA1 gene and one that lacks the gene, were examined. All three accessions exhibited a moderately resistant response to the Pt #526-24 isolate, independent of presence or absence of the RGAl gene (FIG. 17). Similarly, T. aestivum cv. Chinese spring which has several homologs with 79-73% identity, was found to be susceptible to Pt isolate #526-24 (data not shown). It was thus concluded that in the current databases there is no functional homologue of the Ae. longissima Lr/Yr548 gene.

An independent work of the inventors of the present invention and co-workers revealed a similar (85%-99%) Lr/Yr548 resistance conferring gene in Ae. Sharonensis. Unexpectedly, the Ae. longissima Lr/Yr548 gene shares only minor sequence homology with two out of the 31 scaffolds of Ae. Sharonensis previously predicted to be associated with resistance to leaf rust and stripe rust diseases (WO 2021/001832). Furthermore, the somewhat homolog region sequences were found to be aligned to multiple regions within the Ae. longissima genome, suggesting that these sequences are repetitive sequence found in several places and not specifically representing the Lr/Yr548 gene.

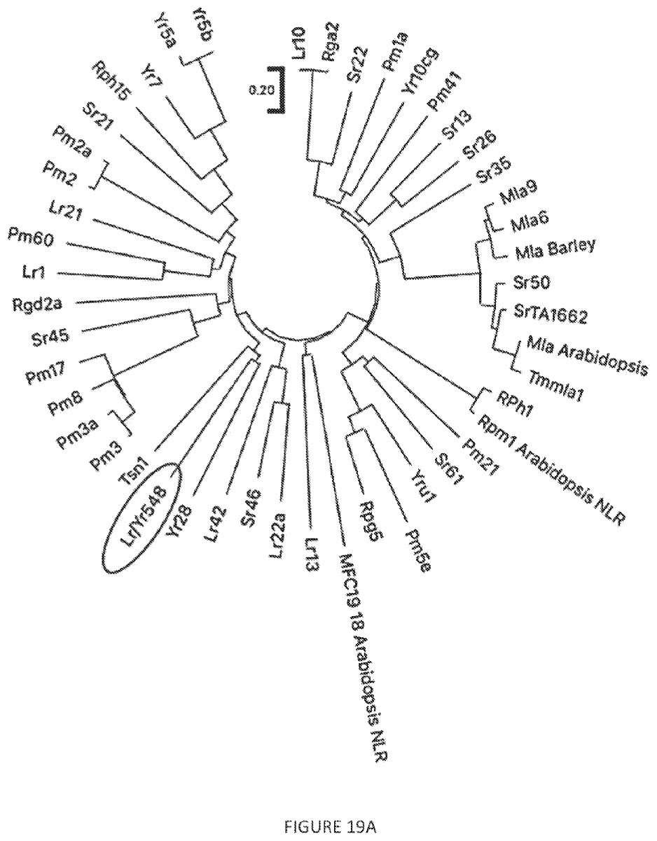

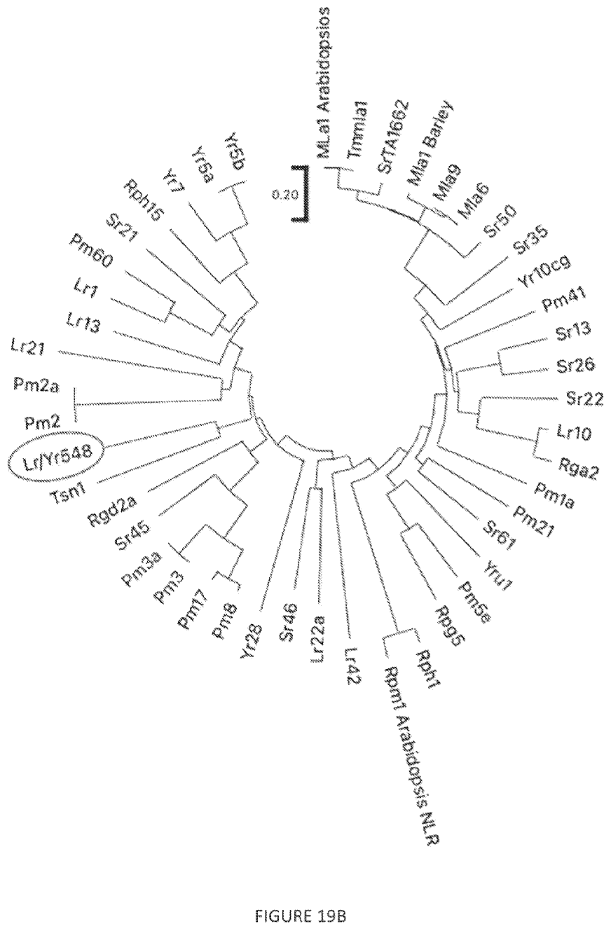

Phylogenetic analysis using the predicted full-length protein (FIG. 18A, SEQ ID NOs: 62-64) or just the LRR and NBS protein domains (FIG. 19A-B) and previously cloned NLR genes showed that the predicted protein is closest to the wheat Tsnl ToxA sensitivity gene, however no close homology was found when only the CC domain was used (FIG. 18B), indicating that this domain differs from the common CC domains.

Next, raw RenSeq reads of 246 Ae. longissima genotypes from the current study, 193 Ae. sharonensis genotypes (courtesy of Dr. Brande Wulff), and the new Ae. longissima reference genome (V2) were used to generate an SNP matrix of the coding sequence of the Lr/Yr548 gene. The final matrix after filtering of missing data contained 57 Ae. longissima genotypes and 72 Ae. sharonensis genotypes with a total of 300 SNPs. A bootstrapped maximum likelihood tree obtained from the genotype matrix revealed high degree of mixed clades of the two species (data not shown). Additionally, many clades contained genotypes from geographically distant collections sites, and likewise genotypes from the same collection site were often positioned on different clades. This pattern is a clear indication of multiple events of horizontal transfer of the Lr/Yr548 between the two species. Maintenance of high diversity within each local population was observed by frequency-dependent selection (FDS), which favors rare alleles, thus increasing diversity within populations. FDS is typical to R genes in wild populations (Bergelson et al., 2001; Sela et al., 2011; 2014, Han, 2019). We superimposed available data of leaf rust resistance on the tree and found that 57 of the genotypes were resistant, 18 susceptible, and five had mixed reactions. Most susceptible genotypes were clustered in a single branch with a high evolution rate, suggesting loss of resistance at the root of this branch and relief of the selection pressure.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A Triticeae plant comprising at least one cell comprising a heterologous polynucleotide encoding a polypeptide having at least 80% identity to the amino acid sequence set forth in SEQ ID NO:1, wherein the heterologous polynucleotide is capable of conferring or enhancing tolerance and/or resistance of the Triticeae plant to at least one rust disease.

2. (canceled)

3. The Triticeae plant of claim 1, wherein the heterologous polynucleotide comprises a nucleic acid sequence having at least 70% identity to the nucleic acid sequence set forth in SEQ ID NO:6 over the entire length of the polynucleotide.

4. (canceled)

5. (canceled)

6. The Triticeae plant of claim 1, wherein the heterologous polynucleotide comprises a nucleic acid sequence having at least 88% identity to the nucleic acid sequence set forth in SEQ ID NO:3 over the entire length of the polynucleotide.

7. (canceled)

8. (canceled)

9. (canceled)

10. (canceled)

11. (canceled)

12. (canceled)

13. (canceled)

14. (canceled)

15. The Triticeae plant of claim 1, wherein said plant shows the phenotype of enhanced tolerance and/or resistance to at least one rust disease compared to a corresponding Triticeae plant devoid of the heterologous polynucleotide.

16. (canceled)

17. (canceled)

18. (canceled)

19. (canceled)

20. The Triticeae plant of claim 1, wherein the rust disease is caused by a fungus of the genus Puccinia.

21. The Triticeae plant of claim 20, wherein the rust disease is selected from the group consisting of leaf rust disease, stripe rust disease and a combination thereof.

22. (canceled)

23. (canceled)

24. (canceled)

25. A seed of the Triticeae plant of claim 1, wherein a Triticeae plant grown from the seed comprises at least one cell comprising a heterologous polynucleotide encoding a polypeptide comprising an amino acid sequence having at least 80% identity to the amino acid sequence set forth in SEQ ID NO: 1, wherein the heterologous polynucleotide is capable of conferring or enhancing tolerance and/or resistance of said Triticeae plant to at least one rust disease.

26. A method for producing a the Triticeae plant having enhanced resistance to at least one rust diseases of claim 1, the method comprises introducing into at least one cell of a Triticeae plant susceptible to the-at least one rust diseases a heterologous polynucleotide comprising a nucleic acid sequence encoding a polypeptide comprising an amino acid sequence having at least 80% identity to the amino acid sequence set forth in SEQ ID NO:1, thereby producing a Triticeae plant showing a phenotype of enhanced tolerance and/or resistance to said at least one rust diseases compared to a corresponding control plant.

27. (canceled)

28. The method of claim 26, wherein the control plant is lacking the heterologous polynucleotide, said control plant is susceptible to the at least one rust disease.

29. (canceled)

30. (canceled)

31. (canceled)

32. (canceled)

33. (canceled)

34. The method of claim 26, wherein the rust disease is caused by a fungus of the genus Puccinia.

35. The method of claim 34, wherein the rust disease is selected from the group consisting of leaf rust disease, stripe rust disease and a combination thereof.

36. (canceled)

37. (canceled)

38. (canceled)

39. A method for selecting a Triticeae plant having an enhanced tolerance and/or resistance to at least one rust diseases, comprising the steps of:
a. providing a plurality of Triticeae plants of claim 1; and
b. selecting a plant showing an enhanced resistance to the at least one rust diseases compared to a control Triticeae plant or to a pre-determined resistance score value;
thereby selecting a Triticeae plant having enhanced resistance to said at least one rust diseases

40. The method of claim 39, wherein the control plant is lacking the heterologous polynucleotide, said control plant is susceptible to the at least one rust disease.

41. The method of claim 39, wherein selecting the plant resistant to a rust disease is performed by inoculating the plant with the respective fungus and selecting phenotypically resistant plant.

42. (canceled)

43. The method of claim 39, wherein selecting a plant resistant to the at least one rust disease is performed by detecting the presence of the heterologous polynucleotide within the genetic material of the at least one cell of the plant, wherein said heterologous polynucleotide comprises a nucleic acid sequence having at least 88% identity to the nucleic acid sequence set forth in SEQ ID NO:3 over its entire length.

44. (canceled)

45. The method of claim 43, wherein detecting the presence of the heterologous polynucleotide is performed by amplifying at least one marker by a pair comprising SEQ ID NO:14 and SEQ ID NO:15; a pair comprising SEQ ID NO:16 and SEQ ID NO:17; a pair of primers, the pair of primers is selected from the group consisting of a pair comprising SEQ ID NO:42 and SEQ ID NO:43; a pair comprising SEQ ID NO:44 and SEQ ID NO:45; a pair comprising SEQ ID NO:46 and SEQ ID NO: 47; a pair comprising SEQ ID NO:48 and SEQ ID NO:49; a pair comprising SEQ ID NO: 50 and SEQ ID NO:51; a pair comprising SEQ ID NO:52 and SEQ ID NO:53; a pair comprising SEQ ID NO:54 and SEQ ID NO:55; a pair comprising SEQ ID NO:56 and SEQ ID NO57; a pair comprising SEQ ID NO:58 and SEQ ID NO59; and a pair comprising SEQ ID NO:60 and SEQ ID NO:61.

46. The method of claim 39, wherein the rust disease is caused by a fungus of the genus Puccinia.

47. The method of claim 46, wherein the rust disease is selected from the group consisting of leaf rust disease, stripe rust disease and a combination thereof.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A DNA construct comprising an isolated polynucleotide encoding a polypeptide comprising an amino acid sequence having at least 80% identity to the amino acid sequence set forth in SEQ ID NO: 1, further comprising at least one plant compatible expression regulatory element.

62. The DNA construct of claim 61, wherein the isolated polynucleotide comprises a nucleic acid sequence having at least 70% identity to the nucleic acid sequence set forth in SEQ ID NO:6 over its entire length.

63. An isolated promoter sequence capable of deriving the expression of a polynucleotide within a cell of a Triticeae plant, the isolated promoter comprising a nucleic acid sequence having at least 90% identity to the nucleic acid sequence set forth in SEQ ID NO:4.

64. (canceled)

65. (canceled)

66. (canceled)

67. (canceled)


SEQUENCE LISTING (68 sequences)

SEQ ID NO: 1 - protein, 1510 aa, Aegilops longissima
MSGVGEMIASAVVKQITSRLIKLLEDETTMQWKFKGEVDKMAEKMKYLAAVMHDADDKVRRGGRVGETVGLWLLKLKSISHDVEDLLDDLDTKNNEAKLKVFFSGNNQAYRWITRPHRLKDVGKRIEEIKKEGCEFNLDPKAQVEQRQEETYATISATTGMQGRSAEKEKLINLILRSQANERISIIPIVGLGGIGKTTLARSVWADEKFNIFDIKAWVHVSDKFDLHKIARTIIRSVDTDINLDSNDLHFLQNILRVKLAGKKYLIFLDDIWEEDGDKLDKLKDMLQHGSNGSRIIITTRNQRIVETMSTGILAHERVILPVHESDRIKLGVLPTDDCWELMKIRAFGPDDDQTPLKQIGYEIASKCGGIPLVANAFGKVMSGNKSIAAWQYIKDRMVDLGLRDEDQKDTLESLKLSYYFMKLEFKICFSYLAAFPKGFIIDTNRLIQQWNALGYIYSEHDGKNCIDYLLGMSFLQISGPSSASQSPACANASGELTMHDLVHDLALVITVDEFLVLDADATGPRTWKKAIHCRHAELIQYKNQSNVFEHLPKKLRSLHIRDSLKWKLPPKAFSRCKYMRVLDLACLVGQLSEGQSTSSNMRLPRSINKMKLLRYLDATGLPVSSLSKSLHILQNMETLIMSNCSLETLPDNVCSLHKLSYLDLSGNRSLNKLPTSLGKLSKLSFLNLSGCSVLQGVPKSICQLTCLQYLDMSECRAVQNLPDKFGSLPNISFLNLSGCSKLTKLPDIVNLESLEHFDLSSCHELKDLPQDFGNLQNLVFLSLSDCYKVSVLPESFCQLIHLKYLDLSDCHGLRELPECFGNLSELDSLNLISCSKLQVLPDSFCSLFKLRHLNLSYCMRLEKLPSSLGDLKLRLLDISAATFHDLPDSIRGMTTLTKLVVSTAQLGVHNKVRDIRQHLNLPGEIVHTVHLTENKGCSSIVELAQLTCQNLKVLQLQNVRHPEDAERAKLRDKSDLRVLWLCWGLQREEDIYVLERLVPPRNLEHFTLDGYMSKDFPNWISHISTYLPYLTFLTLCNLGACDNLPPFGQLPNLRCLEMNYIPNITKIGKEFYGEGGTCRKLRSIQLKSMENLVEWWTALSGEKNEESLIPNLHCLSIRNCPKLKFLPYPPRSMFWHLDNSSMVSPKEGFGKLSSYAFPFLMVVRNCNFAPDKWDWLQHLPTLEIFYVESINGLRALPEAMQCLNSLTRLNLWSLKDLEKLPEWLGDLTSLEYIHIKDCPMVTSLPVRMKCLAALRELCLAECRGLEVLPEWLGHLTSLQEFYIMSCPNLTSLPESMKCLTALRVLRLMECEGLEVLPEWLGHLTSLQEFYIIRCPNLTSLPKSMKCLTALRVLWLTECEGLEVFPEWLGHLISLQEFYITCCSNLTCLPVSMKRLTALRLLWLGQCEGLDVLPEWLGQLISLQEFNIVGCPNLTHFPESTRNLTALKKLNIWGCPSLVERCQGEDADLISHIPEVLLEGKTRELWSTSGDAEASTSGASTAEEAGPSTS

SEQ ID NO: 2 - DNA, 17127 bp, Aegilops longissima
  source 1..17127: mol_type = other DNA
atgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaaggtacgcatgctagctgccctacgctgtgttgaagcaaaattagttgttcattttttttagatggaagccaaattctttattcgtccactaggcttgttttgggacagagccccttctatcctcatggtcggagcctcttctattctcttatcttcggtgtagtctaccctcagggccggtcctaggattttaagggcccggggcaagattagagctcggggcccattataggattgccgtcagactgatttcctagttcagggtggacggcacgagcggtcttttggaccggctcgtggcccgctcagtccgagctggctcggtcctggcctgacaaaaaaatcggccggtgcggtccctcaaaacaagaccgaaaatcggccggtccggtccggttaaagctcgggccgaccgagcggaccgaagaagcagccggcggcatggcggggccatggccagagcggggcggggcgggggcgtggctgggggcctgtcgggggcatggcggggcatggcgggggcttggcgagtgcgtggcggccaaggcgtgacagggccacgcaccagtgtcgcggccgccgccgtccaacgcacaagcagtgcaccaccgcgtaacaccatggtcaccagccttccggcgacgaaccatcatgtctaggagttcttccgtcgtgagatctacatctccgtcaaaaaaatcgacgagggaggtcctacgtagccgcatcagccatcgttcctttcatctctgcccaccggccgtcgtgtcagattcgtcgccgatgagcttcctccggtgttcttcttggtcacgtagcttcctctgatgcttctcaagctcccagacatcttttcgatgagctgcgcttcctctagcaagatatctctccgatgagccgtcgctggagctctagagcgcgctctgcccatgtcttggccggtcctctcggtcctgcccgagctcgtccattgccggtccggtccctgaaaacaggaccgcggcgttgctcggtccggtccggaccggaccgccggcggccgatccaaacaccggctcggacagggaccggaccggctcggaccgtgtccaccctgattcctagttgtctttcctgcttcttgttcgaccccaaaattcaatcatgcacccaacacccgatggaattgttttactcgctaggaggcaaaattgaaagtactatcaaaaccagtgcatttatagaaaaatagattaaccataatcatggactagtttctttccctggattacggctttgcatctgtgtgttccatggaatattgcaaagaacatacacaatccaatcaatttaatgggaaagcatctatatatacattgcaattagcaccggacgtactgactcctagacaaatgccatgtaatttttattatttctatatatattttccctaaacatcacatcaaggtctctatatcggacatgcccttagtatttgaaatttagcacaatgctttttattaactcgataatacctaccgcaaccatggccaactaatcaattaacaatgccccatgacaataacgaagatctaatattgattaccatttataactttttattaattactatgggaaaagtcaaaattgtaaggcacaataccttgtctaggatcagcttctcgtctcgttgatggggagtcgccggctgaagtagtagtggcagcatgctgcatgctcggtggccatcgcttgtccgccgttgttcaattggttccgacggccgcgaggcgaagaggcaaaaatccatccggtggcagccgccgacaccgtgcgtgcgtgaagtcgtaactggtgtgcctctccgagatggctgactcgatccaagatccgagtagtaattagtagccagccagccacgtagtaactgggaccaacggaaacccagcagccggcctacatacatacgtcctgggtgggggcccctgcgctccgggggcccggggcgaccgcccccctgccccccatcacgattgggcctggtcctggttcgtcacggcctgggaccaaaggcccaccacgtggcacgagcccgtgctaggggttaggagcctttagtcccggttggtgacaccaaccgggactagaggtttaggggtttagggttttatgatttataatttatggtttattttttcttttcgttttgtgttctccatttaattgtttttcatttcaaacatatttcacgctgccactgcttctacataatgtacatgtcatatatgatcaaatttcacgtacgaccaatcatatatcatagaatttctcgtacgaccatgcgcgcgtgcacttacacatacaatttgctgatcattacatgtcgcccatgtaatggtattctccttctccgtctatgacctggtcaagcaaaaatcctgctatttcctcttgaagtgctcgtatgcggcccgttggtaggagttgatcccgcatctcttgcatcttgaaagaaggagatcaatatgaatacattagttgtgtgtatatatattagatcatgattaaaaagaattgtncgccgccgccggagggtgccaccgggcaaagcccgggcagcgtcggggctgttcctctccctctcaaggcgcgcggcagcgcgggctgctgggcttcgtggaggggctgcgcgcgtcaccgacaatggctcgctgcagcgtcttggtggtgactgctgcatggcaggcttcgtcatatggatggcgtagcatggcgtggcggcagcaggagatcaggaacaggggggtggtcctgtttcatcggggcagcagccagccatggtcacgtcgacgatgtggtgaccggatatagagatggtgatggctttctgctctgcaaaatttttgaagtcggcggcggtgtgggacgcccctctatggaccacggagcttccgaggtcggtgccggtatgggacgctacctacttccggcatcttctccggtggtatgggacagtgactgcttcggcgataaatattttggtaaagttgttgcttgtttgctcccgcgagcagccgagggagagaccatagccaccacctctacctctcctcccgccccacttcccctcaccgcccggagggtgccactaaggctggacatgggccagctcgggctgggctcggtccgagccgcagttttttttagaaaaggaggatgacccccggcctctgcatatgggcgatgcatatggccactttattaataattttcacaagaccttacaaagtcatacaacagtaagactaaagccgccgtctaagcaacaaactgtcgctacacctatccagttaagaaatgggcgcagatagcctgggcctaataccaaagagacatcgtatccaagcctaacatctaagacccgagaccccaacctagccacttgccgggtctggggcatacactggtcctgcgtgctctcagaggccgccgccaactggcaccgctccatcttcagaactgtactgatgcatcaaccttgcttggtctagctatcatcgacgccaccacggtgcccaacgacacctcctccctgcgcgcaaacagctgagcacgtcgcggccgccactgatacacctcagctccatgctgccaagtaccaccaaccgacacagcttgaagtccttgaaagatcaatcatgtgtagcacctgccgaccaggcatgacaaagcgtagcacctgtaggtcgggcatgacttgacatctccatcgaagctccgtgcacctgttcgagccgcagttttgctcgctgagaaacaactcggctcgggctggcttgcttgacccatgggctggaaaatcaagctcggctcgaactcgctctggctcggtcaaactcgagccggctcgcgagccctgaaggaaacagtcactagtcactgacaatgggacccaccttatcttcctctctaagctcatgcctgtaggtcatgggacacacgatttcgcccaaattccccagccctacagcagcgcaccgcacctgcagcagcctgccgcccaaattccgtcgtgaccaaggctggacacaagcgcaccgccaaaattctgctagttgggagagaaagatcgagcgagcgagcaccgcacctgcagcagcctgcgccgtcctctcacgagggctctgcagccgcaagcatcacgccggccccgcctctgtggttgctgccgccgctcgtacgggccatgccgaagccgcagagatgaacgaggggtcctccgctgcagagatgaacgagggatacagagatagatcgagcgagggagaccagagatggagtgtgtgggctcgcctgtgcgcatgcgtgcatgaggccacaaattcttcacgggccaaatgggctggaccgagccaaaagctcgccgcggtccaagattttgggctcgactctgtccacgcctatctcaggccgagcccagctcgggccgagccaagatctcgcccccgagcgtccaggcttaaggtgccaccggggaaagcccgggtggcgccagggccatgttcctctccctcgcaaggcgcgcggcagcgctggctgctgggcttcgtggagggggctgcgcgcgtcactgacaatggctcgctgcggcgtcttggtggtggctgctgcgtggcaggcttcgtcatatggatggcgtagcatagcgtggcggcagcaggagatcaggaacaggggcggtgaccctattttatcgggggcagcagccagccatggtcatgtggatgaagtggcgaccggatatagaaatggtgatggctttcttctccgtgaaatttccgaagtcggtggcggtatgggacgccccctctatggaccgcagagcttccgaggtcggtgacggtatgggacgctacctgcttccagcatcttctccagcggtatccgcctagtgagatgtcctcagccgacagagggattgatgacagtgattgcttcggcgataagtatgcgcgcactctggtgaaaacacaagaactttgaccaggcaatgtcgatgcgtctacctcgtgcccttgatgaaggtggtggctcgaagctcagccttggatatgaaagtcctgagatcgacctacggttggactcgtcatcatcagcgcacatgcgatgaacccttcttgaagggcttgactaggagttgtgtactttttgttgtttcggttcttgtcaaagggttcgtggtaagtttgtggagatactgttgagaggtagacatcctcgttttttcgttttttattctacttgtttggcatcgctgcctggttttgcctgccttttttgggtaatggataatatatagctgtgtgcatcaaccgatgttgaggccaggggttttcctcctttacgaaagaaatttgtgccttagataagttctcgactcacaagaatatgtcacttaacaaatcttttttttttgtctacagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggttagtttctaacactaactctattgtttatgttcggtcattgatgccttatctattagctggttatgatattttttattttgatagggaatctaatcatatactaccacagctaggatcctggtttgcgtgtattataatctaccctcttttattattagtttcccaagtgttcttttcatataattgacatccctagacgttaatttcacacaaccaccatatcatcatggataaagagacataccaaatgaattcgtgtaaaatatataagcccgaacggaagtttgaagaaacattcactgatgaaacaacactggaaagaaaaaaaattggatggctcctgacccacaccgtcctctggacctctcgacccataaaccctcccgccggtgacggaccaccaagccgcgccgacagatcaatccttccaccccctccaccctccattctccatgaaatcgctctctactactttgacgatgaattgccttcccatgcttccctgcaggctttcctcggggtcggcaacgccctgggcatcgccttcgaggaaacggcctggcttggcaaaagcttgccaatctccatttgtagcccatcggaacccacttttctgatgttttcctccgtccacaactccatcatcatttttgatgagctgcttatggcagttctgatccatctgtcattgggggagtttccgcttctatgtcgtctcacccaaaatcatgaaaattgttttatcccaagcatcgatctccaccaaatccttcaactagctcttcaaccatgtttgcgtggctccttctgccacgaccattcaaccgcctctctctacctttggctctctgggcggtgcaaagcttgcacccaccaattcagggggtaagatgctctctgatgtgcctcatgatccacagccggcttcgccatcaccaaacttttactacaagttcggtgggttgtctactttggccgatgggccttcggctcctcaaagatctgcttcaagttagcacaaccaaatccagtctaccttagcgtgagatatgtgacgttggtaatgttaagttcttgggtgcccaggtttaagctttcggttctttattttacagaattaccactcagaggtagttttttctctgtttgctaacaaacattcaattaaatctacttgcatttggattagcttcattttagcaaagggaaaacctaatgcgtttatcgtaaaaaaacactttagaccaccttttctctaggcttaatgctttcaatgtctcttgcgtccatgattatatatgttttcctcacctacatgtcctcttctagtatcagcactgaaattgtcaaaaatctcctctaaaactacctgggttaaccttccacctctccatttgccctgaacctacacgccaaattgttatttcaaacactgcactcaatcttgttctctctcctctccaagcccagcccaaattaagatccctccaacgtgcccatcttcataatgggcctgttgcacgacacgtgcctttggcaaaagtttttgcatgtgcgtgttcatcgtgatttgatgtgatcgatcctgtgggggaattccaacgattggtatcagagcaaggtcgaggtgacgttgcgcttgccccgaggtggcgacccgactagcacctcggggtgggcaatgaagtcactaggtgatgcgggcgatatggtcgctggatagtgcagcgacgaggaggatcgggcgatcgtcgttcggcggagcgacaaggagggggacggcaggctgtcgtcggcaaggcggaggaaggagatcgttgacgggagtggtggtgccgaggtgcggtaccggaagagtcgtcaagatcgtcgtccgcggggatagatgagttgaagagtcgtgcgagtttgcgtgactggttgtcgtcgtgtcagtgagtcgtcgtcatgtccaagtgcttgtctctctgaggattgttgaagatgaagcgtgtcatgttcgatgggctgtctggtgtggtgtagcaccaagggcgaagacgtgaagtggagatcaagctcaggcgtatagtgggacgaagaccagcgaaggggagcctcttcaatgaggacctgtctctacatgggacgatgtgtgatccgttggcatgcattgcgttaggggtgaccggtggtgttgcctggcagcgtgactgagagtctgaatcatacgttgaggggatcgtcaagttgcggatctacaacaagtcaaaatcaggagaagtgcgaggaagatgacaagtcaagattagaggaagattgttagcataatcttgattatagattaattagttagtgctagttagatgctactttgctggctgccgtgtagtagtccgtattggagtcaacattgacgtacgtgtgggttgtgtactagatcgtgtccggcttgtaattagcttgcatgccgagtcgtcatagtctgtatgagcgttggagtctagtcctgttaggattgttgtcatcgtgtgatagtaggtttagatcatggtcggttgggcatgtatatatatacagccctgcgtgagggttttgtaacgacacccaaaaaacaagaaaaggaaataaaagaaaagcggcacgacacgtgcctttgacaaaaaattttgcgtgtgcgtgttcatcgtgatttgatgtgatcaatcatgtggggaattccaacagggccaaccgcctgcatgccagccacctgcctatgagtagaccaaaccaacgtggctaactacgaccatcgaccgaggcaggcgatacgaattcccatattttccacgttcaaaacacgcaaacagcacttccctctcgccatctgccatctcgatgggtcaaaaaggggatgtgaaattaagcaatctcctccacttcaatggagaagatcaatgaactacccccctgcatgtgctccacgagcacgaaaaaatcaacgaaacttgtaaccctacggccagcgctgtctagtgctcgtcgcccggaggggcagaacagatgccgcagtcgccgggccaaatgtccagtcacgatatttctgcctcaccatatcgatatgctctgctcaactctgtaaatggtaaatctcagtgaagatccagctctttctacggcgtcctcctccgcaaaaagcgaagtgcaccatatctaactcctggccatgcccctttacctcgctgtttccgcaattatttttatggtgtagaagttcctgaaatcatgaaagaaatactggaacactatgaccatgatgtaagatggtctcatggtgggattgcacaaaacatgactttatgcgccctcaacgggaataaaaacagtgatacatccatatatgtgagagtgagctgaatttttttcccggggcacctaaattcgtgttttcgcgatcactctgttggatctttttatgttattaccaaaaaaggcttttgcaccgctttatatataaagcaaagatcaacatcatccgatacaaacgcacgcgaacaccacacacacccaaggcaggatacaaaagtgctgagcacagcaacaccacctctagcacacaccacgaagaattgaagccgcatacaacgaaccgtggactccaaggcggtgccttcaggaaggatacgacaccggagcgccgccactagggctggacacgagccgagtcgagccgagttcggtccgagcctggctttggctcgccatgaacgagccatgctcggctcggctcgctggttgaacgagcccgcatctggaggctcggctcgtcttgcctatggctcggtccagcccgagcagctcgttttgcctctggaggctgggtgcgtgggcgtacgggcgcatcaggcgaggctggagcttgacggcgttgaggggagcacgacggagacgtgccggcgtggcgaggtcgggcgtctggtgcgtcgaacgcaggtggctagcctccgtagcagttgatcttgcttcctctcgtacgtgtgcatccagccaactccaggactcaatcaacgtgcttcaagttttgtacttgtgtccaagaatcaatcaacaggctggtgatgcgtgcgtgcgctgcggccgctggatgcgatcgtgcgtgccgacgccggcggcgggatgcgtcgtggtggcatgcaggaaatagcttggcgagaagcgtggggcggcggcggcggcgaaccctagcgggaggagacagtggagacgacgcgtctcgctgggttgggctggttgcgctcctctactgggctgcgtgattgcgtcgcgtcctgggcttcggcttcggacgagccaccgagctggaccgggccaaaacaggctcggctcggtcggccaacgggcccatttcatgtggctcggctcggcctatttagctctcgggccgagctgagtcgggccgagctggagcgagctccgagccgagcccattagcccgttcggacgtccagccctagccgccaccgcccgatccgaggatcagagtttcccccggagccgcacgacgggcggtgagagccgcgacgacgccttcaagaagggaacgagcttcgccatcgccggtccgtccgaagatagagcaggttttcatcccgaccaacactcaccgtcgccgaatgctacaccccggcaaccacgccgcccacatggccatggtcaccaggcagctccaagacacgggctctgcccaagagcaccgtgcactgccaccagggccgccgccgaggcatccaagaccttgacaccaccgcacccgagactcgccgctaccccaaccaaatagacgagcagaaaaggatccgcctttgcacccctggcccaccctcagcatcgagacccaataggtcggccagcactggcatccatcgacccatcctgcagcccgaagcgtgagacgagatcggtcctgcagtaccgtggggcggggacgaggtcagtcctgctgcaccgtgcgcgagacgagcttggtcctgcggcactgtgagcgagacgagctcggtcctgttgcaccgggtgcaagacgagcgatggaccgcagctgggaaagggccagcccttcgaagggagtagcgatcgagcgtcgaggagatggagtgaaggccaaaacggtctgatcgcacacaagccgacgccggagaagctggccgccgccgcaagcggatccgccaagccaacatggagccgccacgacacctggagtccaccaccgccggacctccacacatcgccgcccacggccggagcaacgtccacgcccgaccgctgccctacccgcgtctcccggcaagctcatagcgccggagccgtcgggcatgcaccaccgccaccacgcgccaccggccgacccccaccaccagatccggctggatccagcaggggcggaccgcgcgccacctcctgagacgggagcgccgcagccgccccgccacgcacgagacccagggcaccggcgcgccgccaccagccggctgccccgcgagcccgccgccgagttgctgaaccccatgaagtccagtaccacagctcgaggaggccaccctgcgccggcgtcccacgagcagggagggagaagggccccgccgctgccgcgcccaccgggctttgaccgacggagctcgctggcgacggcggggaggggggaggctttttggtcatttgcccgacggtacctcgccggcgacggcgggggggaggggaaggggggtctcagaccctctcactagaggcgctccatcgcccctgtctgatcttattaaagacggggaagcggcttccgggctgccgcctatgcgaccgccgggaaatctcaactggcgaagacgaacccttgcccccggggttgccctgtgttcgcacctcccccctctctctacgacccccgtgctggtctcgggctctttgggggaatcctggaccctccacattccgtctctcttattgtccccccgtccccttccccccccccctttgccccccgcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggccctttctccccccctttatccggtctcttgccgatggacgccctccaggcgccgcctcccccgtcccccccccccagcgcgcgcgtgcgcgccgcgggagggggtagggggaggagagaggggaggcgaggggcagcggggcgagcctggaggcgctcgccctgcggccggcggcggcgggatcaggagagaaggccggcggcggcgggaggggaaccctagcgggggagcgggggagagccttcctttttatgttattgtgatgatgttccagtatctttttctcataattattgagaaattttaacccgttgaaaccttaatgggttcccagacactaggattattgcaatttaatgaatgaattactgtggaaattaaattatcatatatccacgttttatttatgtaaaagtagtactattatgtatccaaaatttaagttcttttagatcggtaccaatcaaacaattctgatttcgaaaacatgaaactgatatcattacatgtcatgaactatatattttagtaatctataattataattatgtgaggcaacatgtttttgcatagaataaaatggtcagagcactatctctaagatcgtgtcaatgtccaaaacagcttacattttgaatgataggtactatattctctgaacatattattgcatattaacatctaatcgattgttgcataacgattaatcacatggcaactatacgctctaaataaccattatatatatgaaaatggcatagtaggggcattcctactgtattttaaaaaaccaaatattatgcgctaattctgtccaatgtgttttgctttcgttctacaacacgttttgacagtagttacttctacaggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagaggtacggaactccgaagcccagtaattttcgttcttcttagctactgtctgcaaaactctagcacttaggggttattgacaagaagggtaacaagaaacaaatcttgttcttctgcagaattgtggtcgacttccggagatgctgaagcatcgacttccggagcatcgactgctgaagaagctggaccatcgacttcttga

SEQ ID NO: 3 - DNA, 4533 bp, Aegilops longissima
  source 1..4533: mol_type = other DNA
atgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagagaattgtggtcgacttccggagatgctgaagcatcgacttccggagcatcgactgctgaagaagctggaccatcgacttcttga

SEQ ID NO: 4 - DNA, 4091 bp, synthetic construct
  source 1..4091: mol_type = other DNA
agtctctatccatagtttctgtaaatattacaacatggcaatatacaaccatgagagatggataagtggaaaacatagacttaatcacaataaggaatcatatgcatatgagcaatggattagtggaaaacatcatgcaaagttgacaacttttataacagccagttccagacgatcgtcgaggactcctcccccctcatgtctgatgagtcgtcctatctttcgagagagcatactttgcggaccgcctctctcatgcttgtccggcctgcaattcacgttttcgtatgcctcaaaatcaaagttatcggggttatgttttaacgaccccccccccctcatgttgaagttatcggggttatgttttaacggccccctctctcatgttttcatatgcctggaaatcaaagttatcggggttatgttttaacaacaagatgaatacaagatttatacacaagataaatacaagatttatacacatggatgaatacacaactcaacaaatgaaccaaccatcatcctatggacaaaactcaacatgcaacaaatggacaccttttgtcgaacaacggagggatgatcagggaccaactccgcggagatgcaacaccacaacctgtaattccaccgattgaaaaaaataggaaaaccccaagtgtagtagcgcttgcatggcaagaaaacctagtaaaacctagtaaaaagaagtatatttgtcaccaaaatacaacacttgacatggcgaaaaagcaatttttttataaaggagaaaatagatcctgttgaacttgagcatttctcggactttgaaatgctcctgttgaactaaaactatttcaaatgaactagacagaaattttccatattacttctaccatgccaaaaatggttctccctaggccaaaagcttgacagaaacttgacaaaaattgcattagaacttgtccatgatctcatcgatcttgagcagagtagagtatattgtagtagagtataactaagaaaaaattcaccattcttaatatgtgtttgtgacatcactaaagaagatttgaagcaccatcgaacaaactaatgaacatatctcctaataatacttgtatgctgcacaaaacaatcactttctacaaaagacttttttgtgagcaacatcaaagtagagaacatgattaattatagcctttttccttttgcaatttgtcatgccattaagacctatgcaacaaaagaccacatgcttgataacaaagccatgaatacaaattgaagtcagttaaaagaaccttttagcagaacgattattttaattatggacatcagctagtgaaaagacttgcccttccaggatttaacctttattcagtaggtttttaacacaatgaaaacaaatagttgatagtggacttgtggatacatgagcattcaaaattttcagaccacaagaattaggagtgctttaagttcatacattgcccaaaaaaatgagtactttcagataaaacagtgtgtagaaaacatagaacattcaatgtacactttagttatgccagtgtaaaaaattcagaacattcgattatgagagtgtcaataattgagaagtcagtgtgcatacatatcagatcattcttgtaacaaaactgcaacataaattcagttacgaaggtgtaaaagatgcagaacattcagtgaaagacggtcacaactacagcaaattcagttaacacattcaaatatatcattcacttaacaagttcacagaaatcatggacatatctaattcaatttactggaacgaatgagttcaacacttcatatgatcaacagggtacaatcaaacaccacatccacgaccaaatttgacaactttcaactaaaaaccatgaacacattagcaacatcgcaaactcctcttaactactggcactgaccactgaccacttgaccacattagtacctaaggcaatgagatgacaacacactgctccatactaaactatgaagacaaaactttctatgaacaaaaatttcatctaaggccttgtctgttgcatctgagcatatatccagaagatactttgatcatgaaagaaacattaatatggatgtgggtagccgaaggctttgtcagtgagttccaaaaatttctatgaacaaaaaaaacagcaatgcagcggcagcagtggcaagcagagcaagcagcggagcagtggcagcaggggaatgggggagctcactaggggcggcgagcgatggagctggagggcaggacggcggcgaggggagcacgcaggggacggcgtcggggcaggggcgtggcgacggcgtcggggcaggggcgcggcgaggggagcgggggacggcatcggggcagggctcggggaggcgacaacgaggtccggcagcctggcggcatcgaggtgttcatcggcgccgtcgggagagaggaggagactgaagaaactggatagggattgtaattttttctaagtcctgcttatatagccccaccttttagccctggttggttgctccaaccaggactaaaggcccccctctagtcccagtccgagccaccaaccgggactaaaggtgaattttatcaggcgaaaaggcgggaagcccaggcctttagccctggttggttgctcggaccgggactaaagggggtcctttagtcccggttggaggcgatatgcaagacatgtcgccgaaggagcctcgctagaatcgcgatacgcatgacagcgggcgagatcttttgacacccgaaaccccacatgctcaggaaggaccccatttggacgcacggtggtaatgggctgccctaggtcatttcgcccacccctaggacatcgtagatcgatgctttccaacatgaagaaagtcgaaaaacaagaagcagaaaaactagggcgagagagaaaatgtagaggtttgtagattgttttttcgtttgtgtgtttgttattcaatcggccatcacccctcacgtatgtaagagatagtttgacgttctggacaacgcaagtgcttcgaatcccgtaaaaaaagacccaaaccaagtcaaattcggactgtccggagggtctggatgcccagagggttccggaattatccgggctgtccaatacgggtacagagtaaaacccggatccaggcccggaggctcaggactttcagtcttcgttttggctagctctactcttggatggcaaccttaatgctcatgcatccagcccaaaatctgcctagtactaccagtgtcatatatatttggtcttctgtcgactagggcgtacttccaggagtactaagtatattcccctataattgtattctttaaaatggtacgatgattgagaccaaacagagattggagtgtcgcattagctttttgctggaaacatttcttcctttagaccactcgactagtctgcatcttaattgtattattgattagcacccatcctattcagctatatattggtaaacaagacagagacatgcagtaatttaaaaggactagaaccacgcacagaccaagtcttggtattaacttaaattaaatcgggaggcctggacatgcgtttactttcttcaagtcttcctcaatttgcaaatgtattttgaaactgcattattggctatatggagacaaatcttgggcacgaaccaatcagaccaacggtagccttatctcgtcttccccctcgacgatctcatccactaaccttagagacttggaccgttgatggctgttgcttcgtttggaatcttcttcaatttgcacacaaaccaatcagaccaacggtagccttatctcatcttcctcctctccgatctcatccattagccttggagacttcgaccgctgacggctgttgcttcgtttggaatcttgttaaatttgcacaaaaaccaatcagaccagcagtagccttatcttatctcccctctctccaatctcatacattagccttctgtgtcttggtgatgaccacaagcaagcaaagcgctccttaccttgctgggcttccacaaacacaacaatacccttgcaggacagagcaagagcttgttgtgaagcc

SEQ ID NO: 5 - DNA, 1456 bp, synthetic construct
  source 1..1456: mol_type = other DNA
gacccaatgagaggaagctagctggaagtgatcctgccatatacagagactaaagcaaattaagctgaaatgcaagaggtaataatgagaaacatgtaatgtttttcttcaacgtaactggacagtgatttcatcgtaattggttatgctggtgtattctgacaatctggaactctatgtgttaagctttgtcctgaacatatctggtctttcttttcagtttagagctagtgcagcactacctattgggctggaccttcatgccaatccaagctttattggtggcagagtcggaggcggcagcggcgctatgccccacggaagggatcctgcccggaagatcactcccgatgcattctgatcctaccaacagcggcgatggtggtgcatgcccatccttgtgagctccagttgagctgtaagagtctgcttctcccggatctagttcacctgatggtttcctctcatgcatgtgaataaaataccttgtgctagcaacacaaatattccacagagaatgcattgtcctcaaattgcttgcgttgctctggctaatttcgagctgtgtcctaaaatttatctcatctccttcgtttcacatcacactctcctcaaatttcgtcctggaaagagcgaatcaagaatgacgaacaccatgttggttgcacgactgaactgctaactgttctgcatatatatacttggcactgaccggtactagtacatctcgtgccttttcaggatagagacagaccatccagcaagcagtagttttttaacctatgtaccctggtccagtttttagcataagtaaacacttgcaacaacttttacttgcactattattttcggttattatttactccaaaatacaaaaaacatgtatcacaatatcgtaaaaactcaacaacacacttggcacctccacctgttaaaaaaacgcttggcacctccggatactttgtttggtacatcatgtgaattatattttttttttcgaaaagggggaaaacctcggcctctgcatcagaaagatgcatacggccatcttataaaccaaacaaagatagtgtcaacgtggttcgaaggtctccaaaacagaaaaagtaaaacaagctcacacagagccatagagtggctagaaaccacaactagccaagataagagaccacaacgatgattatactagatgcctatcctattacatgaccgccatccaaaccggttgaatatatcccgagctaccatctcccagcggacagatccagtaaccaaatgctccctggactccgtccgagtgagtggcgaccacgaacggatccgtgccgtagttcgaaatataacctgcaaaaagtggataatgatattctgttaaagaccaaatcatttctacaagtccagatagtccacagcaacgcgcaaaccccaacccgaaacaacattcggtggtttgata

SEQ ID NO: 6 - DNA, 16207 bp, Aegilops longissima
  source 1..16207: mol_type = genomic DNA
atgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaaggtacgcatgctagctgccctacgctgtgttgaagcaaaattagttgttcattttttttagatggaagccaaattctttattcgtccactaggcttgttttgggacagagccccttctatcctcatggtcggagcctcttctattctcttatcttcggtgtagtctaccctcagggccggtcctaggattttaagggcccggggcaagattagagctcggggcccattataggattgccgtcagactgatttcctagttcagggtggacggcacgagcggtcttttggaccggctcgtggcccgctcagtccgagctggctcggtcctggcctgacaaaaaaatcggccggtgcggtccctcaaaacaagaccgaaaatcggccggtccggtccggttaaagctcgggccgaccgagcggaccgaagaagcagccggcggcatggcggggccatggccagagcggggcggggcgggggcgtggctgggggcctgtcgggggcatggcggggcatggcgggggcttggcgagtgcgtggcggccaaggcgtgacagggccacgcaccagtgtcgcggccgccgccgtccaacgcacaagcagtgcaccaccgcgtaacaccatggtcaccagccttccggcgacgaaccatcatgtctaggagttcttccgtcgtgagatctacatctccgtcaaaaaaatcgacgagggaggtcctacgtagccgcatcagccatcgttcctttcatctctgcccaccggccgtcgtgtcagattcgtcgccgatgagcttcctccggtgttcttcttggtcacgtagcttcctctgatgcttctcaagctcccagacatcttttcgatgagctgcgcttcctctagcaagatatctctccgatgagccgtcgctggagctctagagcgcgctctgcccatgtcttggccggtcctctcggtcctgcccgagctcgtccattgccggtccggtccctgaaaacaggaccgcggcgttgctcggtccggtccggaccggaccgccggcggccgatccaaacaccggctcggacagggaccggaccggctcggaccgtgtccaccctgattcctagttgtctttcctgcttcttgttcgaccccaaaattcaatcatgcacccaacacccgatggaattgttttactcgctaggaggcaaaattgaaagtactatcaaaaccagtgcatttatagaaaaatagattaaccataatcatggactagtttctttccctggattacggctttgcatctgtgtgttccatggaatattgcaaagaacatacacaatccaatcaatttaatgggaaagcatctatatatacattgcaattagcaccggacgtactgactcctagacaaatgccatgtaatttttattatttctatatatattttccctaaacatcacatcaaggtctctatatcggacatgcccttagtatttgaaatttagcacaatgctttttattaactcgataatacctaccgcaaccatggccaactaatcaattaacaatgccccatgacaataacgaagatctaatattgattaccatttataactttttattaattactatgggaaaagtcaaaattgtaaggcacaataccttgtctaggatcagcttctcgtctcgttgatggggagtcgccggctgaagtagtagtggcagcatgctgcatgctcggtggccatcgcttgtccgccgttgttcaattggttccgacggccgcgaggcgaagaggcaaaaatccatccggtggcagccgccgacaccgtgcgtgcgtgaagtcgtaactggtgtgcctctccgagatggctgactcgatccaagatccgagtagtaattagtagccagccagccacgtagtaactgggaccaacggaaacccagcagccggcctacatacatacgtcctgggtgggggcccctgcgctccgggggcccgcccccccccccccccccccccccccacgaccgggtctgtcgtatgtgttgaaatattttggtaaagttgttgcttgtttgccgccgcgagcggccgagggagagaccatagccgccacctctacctctcctcccaccttacctcccctcgccgccgccggagggtgccaccgggcaaagcccgggcagcgtcggggctgttcctctccctctcaaggcgcgcggcagcgcgggctgctgggcttcgtggaggggctgcgcgcgtcaccgacaatggctcgctgcagcgtcttggtggtgactgctgcatggcaggcttcgtcatatggatggcgtagcatggcgtggcggcagcaggagatcaggaacaggggggtggtcctgtttcatcggggcagcagccagccatggtcacgtcgacgatgtggtgaccggatatagagatggtgatggctttctgctctgcaaaatttttgaagtcggcggcggtgtgggacgcccctctatggaccacggagcttccgaggtcggtgccggtatgggacgctacctacttccggcatcttctccggtggtatgggacagtgactgcttcggcgataaatattttggtaaagttgttgcttgtttgctcccgcgagcagccgagggagagaccatagccaccacctctacctctcctcccgccccacttcccctcaccgcccggagggtgccactaaggctggacatgggccagctcgggctgggctcggtccgagccgcagttttttttagaaaaggaggatgacccccggcctctgcatatgggcgatgcatatggccactttattaataattttcacaagaccttacaaagtcatacaacagtaagactaaagccgccgtctaagcaacaaactgtcgctacacctatccagttaagaaatgggcgcagatagcctgggcctaataccaaagagacatcgtatccaagcctaacatctaagacccgagaccccaacctagccacttgccgggtctggggcatacactggtcctgcgtgctctcagaggccgccgccaactggcaccgctccatcttcagaactgtactgatgcatcaaccttgcttggtctagctatcatcgacgccaccacggtgcccaacgacacctcctccctgcgcgcaaacagctgagcacgtcgcggccgccactgatacacctcagctccatgctgccaagtaccaccaaccgacacagcttgaagtccttgaaagatcaatcatgtgtagcacctgccgaccaggcatgacaaagcgtagcacctgtaggtcgggcatgacttgacatctccatcgaagctccgtgcacctgttcgagccgcagttttgctcgctgagaaacaactcggctcgggctggcttgcttgacccatgggctggaaaatcaagctcggctcgaactcgctctggctcggtcaaactcgagccggctcgcgagccctgaaggaaacagtcactagtcactgacaatgggacccaccttatcttcctctctaagctcatgcctgtaggtcatgggacacacgatttcgcccaaattccccagccctacagcagcgcaccgcacctgcagcagcctgccgcccaaattccgtcgtgaccaaggctggacacaagcgcaccgccaaaattctgctagttgggagagaaagatcgagcgagcgagcaccgcacctgcagcagcctgcgccgtcctctcacgagggctctgcagccgcaagcatcacgccggccccgcctctgtggttgctgccgccgctcgtacgggccatgccgaagccgcagagatgaacgaggggtcctccgctgcagagatgaacgagggatacagagatagatcgagcgagggagaccagagatggagtgtgtgggctcgcctgtgcgcatgcgtgcatgaggccacaaattcttcacgggccaaatgggctggaccgagccaaaagctcgccgcggtccaagattttgggctcgactctgtccacgcctatctcaggccgagcccagctcgggccgagccaagatctcgcccccgagcgtccaggcttaaggtgccaccggggaaagcccgggtggcgccagggccatgttcctctccctcgcaaggcgcgcggcagcgctggctgctgggcttcgtggagggggctgcgcgcgtcactgacaatggctcgctgcggcgtcttggtggtggctgctgcgtggcaggcttcgtcatatggatggcgtagcatagcgtggcggcagcaggagatcaggaacaggggcggtgaccctattttatcgggggcagcagccagccatggtcatgtggatgaagtggcgaccggatatagaaatggtgatggctttcttctccgtgaaatttccgaagtcggtggcggtatgggacgccccctctatggaccgcagagcttccgaggtcggtgacggtatgggacgctacctgcttccagcatcttctccagcggtatccgcctagtgagatgtcctcagccgacagagggattgatgacagtgattgcttcggcgataagtatgcgcgcactctggtgaaaacacaagaactttgaccaggcaatgtcgatgcgtctacctcgtgcccttgatgaaggtggtggctcgaagctcagccttggatatgaaagtcctgagatcgacctacggttggactcgtcatcatcagcgcacatgcgatgaacccttcttgaagggcttgactaggagttgtgtactttttgttgtttcggttcttgtcaaagggttcgtggtaagtttgtggagatactgttgagaggtagacatcctcgttttttcgttttttattctacttgtttggcatcgctgcctggttttgcctgccttttttgggtaatggataatatatagctgtgtgcatcaaccgatgttgaggccaggggttttcctcctttacgaaagaaatttgtgccttagataagttctcgactcacaagaatatgtcacttaacaaatcttttttttttgtctacagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggttagtttctaacactaactctattgtttatgttcggtcattgatgccttatctattagctggttatgatattttttattttgatagggaatctaatcatatactaccacagctaggatcctggtttgcgtgtattataatctaccctcttttattattagtttcccaagtgttcttttcatataattgacatccctagacgttaatttcacacaaccaccatatcatcatggataaagagacataccaaatgaattcgtgtaaaatatataagcccgaacggaagtttgaagaaacattcactgatgaaacaacactggaaagaaaaaaaattggatggctcctgacccacaccgtcctctggacctctcgacccataaaccctcccgccggtgacggaccaccaagccgcgccgacagatcaatccttccaccccctccaccctccattctccatgaaatcgctctctactactttgacgatgaattgccttcccatgcttccctgcaggctttcctcggggtcggcaacgccctgggcatcgccttcgaggaaacggcctggcttggcaaaagcttgccaatctccatttgtagcccatcggaacccacttttctgatgttttcctccgtccacaactccatcatcatttttgatgagctgcttatggcagttctgatccatctgtcattgggggagtttccgcttctatgtcgtctcacccaaaatcatgaaaattgttttatcccaagcatcgatctccaccaaatccttcaactagctcttcaaccatgtttgcgtggctccttctgccacgaccattcaaccgcctctctctacctttggctctctgggcggtgcaaagcttgcacccaccaattcagggggtaagatgctctctgatgtgcctcatgatccacagccggcttcgccatcaccaaacttttactacaagttcggtgggttgtctactttggccgatgggccttcggctcctcaaagatctgcttcaagttagcacaaccaaatccagtctaccttagcgtgagatatgtgacgttggtaatgttaagttcttgggtgcccaggtttaagctttcggttctttattttacagaattaccactcagaggtagttttttctctgtttgctaacaaacattcaattaaatctacttgcatttggattagcttcattttagcaaagggaaaacctaatgcgtttatcgtaaaaaaacactttagaccaccttttctctaggcttaatgctttcaatgtctcttgcgtccatgattatatatgttttcctcacctacatgtcctcttctagtatcagcactgaaattgtcaaaaatctcctctaaaactacctgggttaaccttccacctctccatttgccctgaacctacacgccaaattgttatttcaaacactgcactcaatcttgttctctctcctctccaagcccagcccaaattaagatccctccaacgtgcccatcttcataatgggcctgttgcacgacacgtgcctttggcaaaagtttttgcatgtgcgtgttcatcgtgatttgatgtgatcgatcctgtgggggaattccaacgattggtatcagagcaaggtcgaggtgacgttgcgcttgccccgaggtggcgacccgactagcacctcggggtgggcaatgaagtcactaggtgatgcgggcgatatggtcgctggatagtgcagcgacgaggaggatcgggcgatcgtcgttcggcggagcgacaaggagggggacggcaggctgtcgtcggcaaggcggaggaaggagatcgttgacgggagtggtggtgccgaggtgcggtaccggaagagtcgtcaagatcgtcgtccgcggggatagatgagttgaagagtcgtgcgagtttgcgtgactggttgtcgtcgtgtcagtgagtcgtcgtcatgtccaagtgcttgtctctctgaggattgttgaagatgaagcgtgtcatgttcgatgggctgtctggtgtggtgtagcaccaagggcgaagacgtgaagtggagatcaagctcaggcgtatagtgggacgaagaccagcgaaggggagcctcttcaatgaggacctgtctctacatgggacgatgtgtgatccgttggcatgcattgcgttaggggtgaccggtggtgttgcctggcagcgtgactgagagtctgaatcatacgttgaggggatcgtcaagttgcggatctacaacaagtcaaaatcaggagaagtgcgaggaagatgacaagtcaagattagaggaagattgttagcataatcttgattatagattaattagttagtgctagttagatgctactttgctggctgccgtgtagtagtccgtattggagtcaacattgacgtacgtgtgggttgtgtactagatcgtgtccggcttgtaattagcttgcatgccgagtcgtcatagtctgtatgagcgttggagtctagtcctgttaggattgttgtcatcgtgtgatagtaggtttagatcatggtcggttgggcatgtatatatatacagccctgcgtgagggttttgtaacgacacccaaaaaacaagaaaaggaaataaaagaaaagcggcacgacacgtgcctttgacaaaaaattttgcgtgtgcgtgttcatcgtgatttgatgtgatcaatcatgtggggaattccaacagggccaaccgcctgcatgccagccacctgcctatgagtagaccaaaccaacgtggctaactacgaccatcgaccgaggcaggcgatacgaattcccatattttccacgttcaaaacacgcaaacagcacttccctctcgccatctgccatctcgatgggtcaaaaaggggatgtgaaattaagcaatctcctccacttcaatggagaagatcaatgaactacccccctgcatgtgctccacgagcacgaaaaaatcaacgaaacttgtaaccctacggccagcgctgtctagtgctcgtcgcccggaggggcagaacagatgccgcagtcgccgggccaaatgtccagtcacgatatttctgcctcaccatatcgatatgctctgctcaactctgtaaatggtaaatctcagtgaagatccagctctttctacggcgtcctcctccgcaaaaagcgaagtgcaccatatctaactcctggccatgcccctttacctcgctgtttccgcaattatttttatggtgtagaagttcctgaaatcatgaaagaaatactggaacactatgaccatgatgtaagatggtctcatggtgggattgcacaaaacatgactttatgcgccctcaacgggaataaaaacagtgatacatccatatatgtgagagtgagctgaatttttttcccggggcacctaaattcgtgttttcgcgatcactctgttggatctttttatgttattaccaaaaaaggcttttgcaccgctttatatataaagcaaagatcaacatcatccgatacaaacgcacgcgaacaccacacacacccaaggcaggatacaaaagtgctgagcacagcaacaccacctctagcacacaccacgaagaattgaagccgcatacaacgaaccgtggactccaaggcggtgccttcaggaaggatacgacaccggagcgccgccactagggctggacacgagccgagtcgagccgagttcggtccgagcctggctttggctcgccatgaacgagccatgctcggctcggctcgctggttgaacgagcccgcatctggaggctcggctcgtcttgcctatggctcggtccagcccgagcagctcgttttgcctctggaggctgggtgcgtgggcgtacgggcgcatcaggcgaggctggagcttgacggcgttgaggggagcacgacggagacgtgccggcgtggcgaggtcgggcgtctggtgcgtcgaacgcaggtggctagcctccgtagcagttgatcttgcttcctctcgtacgtgtgcatccagccaactccaggactcaatcaacgtgcttcaagttttgtacttgtgtccaagaatcaatcaacaggctggtgatgcgtgcgtgcgctgcggccgctggatgcgatcgtgcgtgccgacgccggcggcgggatgcgtcgtggtggcatgcaggaaatagcttggcgagaagcgtggggcggcggcggcggcgaaccctagcgggaggagacagtggagacgacgcgtctcgctgggttgggctggttgcgctcctctactgggctgcgtgattgcgtcgcgtcctgggcttcggcttcggacgagccaccgagctggaccgggccaaaacaggctcggctcggtcggccaacgggcccatttcatgtggctcggctcggcctatttagctctcgggccgagctgagtcgggccgagctggagcgagctccgagccgagcccattagcccgttcggacgtccagccctagccgccaccgcccgatccgaggatcagagtttcccccggagccgcacgacgggcggtgagagccgcgacgacgccttcaagaagggaacgagcttcgccatcgccggtccgtccgaagatagagcaggttttcatcccgaccaacactcaccgtcgccgaatgctacaccccggcaaccacgccgcccacatggccatggtcaccaggcagctccaagacacgggctctgcccaagagcaccgtgcactgccaccagggccgccgccgaggcatccaagaccttgacaccaccgcacccgagactcgccgctaccccaaccaaatagacgagcagaaaaggatccgcctttgcacccctggcccaccctcagcatcgagacccaataggtcggccagcactggcatccatcgacccatcctgcagcccgaagcgtgagacgagatcggtcctgcagtaccgtggggcggggacgaggtcagtcctgctgcaccgtgcgcgagacgagcttggtcctgcggcactgtgagcgagacgagctcggtcctgttgcaccgggtgcaagacgagcgatggaccgcagctgggaaagggccagcccttcgaagggagtagcgatcgagcgtcgaggagatggagtgaaggccaaaacggtctgatcgcacacaagccgacgccggagaagctggccgccgccgcaagcggatccgccaagccaacatggagccgccacgacacctggagtccaccaccgccggacctccacacatcgccgcccacggccggagcaacgtccacgcccgaccgctgccctacccgcgtctcccggcaagctcatagcgccggagccgtcgggcatgcaccaccgccaccacgcgccaccggccgacccccaccaccagatccggctggatccagcaggggcggaccgcgcgccacctcctgagacgggagcgccgcagccgccccgccacgcacgagacccagggcaccggcgcgccgcgcgagcccgccgccgagttgctgaaccccatgaagtccagtaccacagctcgaggaggccaccctgcgccggcgtcccacgagcagggagggagaagggccccgccgctgccgcgcccaccgggctttgaccgacggagctcgctggcgacggcggggggggggggggggggggggagggggggaggggtgaggggaggaaggggggtccgaggctgcagcaggtgaggttcccccctcccccagcgcgcgcgtgcgcgccgtgggggggggggagggggtagggggaggagagaggggaggcgaggggcagcggggcgagcctggaggcgctcgccctgcggccggcggcggcgggatcaggagagaaggccggcggcggcgggaggggaaccctagcgggggagcgggggagagccttcctttttatgttattgtgatgatgttccagtatctttttctcataattattgagaaattttaacccgttgaaaccttaatgggttcccagacactaggattattgcaatttaatgaatgaattactgtggaaattaaattatcatatatccacgttttatttatgtaaaagtagtactattatgtatccaaaatttaagttcttttagatcggtaccaatcaaacaattctgatttcgaaaacatgaaactgatatcattacatgtcatgaactatatattttagtaatctataattataattatgtgaggcaacatgtttttgcatagaataaaatggtcagagcactatctctaagatcgtgtcaatgtccaaaacagcttacattttgaatgataggtactatattctctgaacatattattgcatattaacatctaatcgattgttgcataacgattaatcacatggcaactatacgctctaaataaccattatatatatgaaaatggcatagtaggggcattcctactgtattttaaaaaaccaaatattatgcgctaattctgtccaatgtgttttgctttcgttctacaacacgttttgacagtagttacttctacaggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagaggtacggaactccgaagcccagtaattttcgttcttcttagctactgtctgcaaaactctagcacttaggggttattgacaagaagggtaacaagaaacaaatcttgttcttctgcagaattgtggtcgacttccggagatgctgaagcatcgacttccggagcatcgactgctgaagaagctggaccatcgacttcttga

SEQ ID NO: 7 - DNA, 5033 bp, Aegilops longissima
  source 1..5033: mol_type = other DNA
gtacgcatgctagctgccctacgctgtgttgaagcaaaattagttgttcattttttttagatggaagccaaattctttattcgtccactaggcttgttttgggacagagccccttctatcctcatggtcggagcctcttctattctcttatcttcggtgtagtctaccctcagggccggtcctaggattttaagggcccggggcaagattagagctcggggcccattataggattgccgtcagactgatttcctagttcagggtggacggcacgagcggtcttttggaccggctcgtggcccgctcagtccgagctggctcggtcctggcctgacaaaaaaatcggccggtgcggtccctcaaaacaagaccgaaaatcggccggtccggtccggttaaagctcgggccgaccgagcggaccgaagaagcagccggcggcatggcggggccatggccagagcggggcggggcgggggcgtggctgggggcctgtcgggggcatggcggggcatggcgggggcttggcgagtgcgtggcggccaaggcgtgacagggccacgcaccagtgtcgcggccgccgccgtccaacgcacaagcagtgcaccaccgcgtaacaccatggtcaccagccttccggcgacgaaccatcatgtctaggagttcttccgtcgtgagatctacatctccgtcaaaaaaatcgacgagggaggtcctacgtagccgcatcagccatcgttcctttcatctctgcccaccggccgtcgtgtcagattcgtcgccgatgagcttcctccggtgttcttcttggtcacgtagcttcctctgatgcttctcaagctcccagacatcttttcgatgagctgcgcttcctctagcaagatatctctccgatgagccgtcgctggagctctagagcgcgctctgcccatgtcttggccggtcctctcggtcctgcccgagctcgtccattgccggtccggtccctgaaaacaggaccgcggcgttgctcggtccggtccggaccggaccgccggcggccgatccaaacaccggctcggacagggaccggaccggctcggaccgtgtccaccctgattcctagttgtctttcctgcttcttgttcgaccccaaaattcaatcatgcacccaacacccgatggaattgttttactcgctaggaggcaaaattgaaagtactatcaaaaccagtgcatttatagaaaaatagattaaccataatcatggactagtttctttccctggattacggctttgcatctgtgtgttccatggaatattgcaaagaacatacacaatccaatcaatttaatgggaaagcatctatatatacattgcaattagcaccggacgtactgactcctagacaaatgccatgtaatttttattatttctatatatattttccctaaacatcacatcaaggtctctatatcggacatgcccttagtatttgaaatttagcacaatgctttttattaactcgataatacctaccgcaaccatggccaactaatcaattaacaatgccccatgacaataacgaagatctaatattgattaccatttataactttttattaattactatgggaaaagtcaaaattgtaaggcacaataccttgtctaggatcagcttctcgtctcgttgatggggagtcgccggctgaagtagtagtggcagcatgctgcatgctcggtggccatcgcttgtccgccgttgttcaattggttccgacggccgcgaggcgaagaggcaaaaatccatccggtggcagccgccgacaccgtgcgtgcgtgaagtcgtaactggtgtgcctctccgagatggctgactcgatccaagatccgagtagtaattagtagccagccagccacgtagtaactgggaccaacggaaacccagcagccggcctacatacatacgtcctgggtgggggcccctgcgctccgggggcccgcccccccccccccccccccccccccacgaccgggtctgtcgtatgtgttgaaatattttggtaaagttgttgcttgtttgccgccgcgagcggccgagggagagaccatagccgccacctctacctctcctcccaccttacctcccctcgccgccgccggagggtgccaccgggcaaagcccgggcagcgtcggggctgttcctctccctctcaaggcgcgcggcagcgcgggctgctgggcttcgtggaggggctgcgcgcgtcaccgacaatggctcgctgcagcgtcttggtggtgactgctgcatggcaggcttcgtcatatggatggcgtagcatggcgtggcggcagcaggagatcaggaacaggggggtggtcctgtttcatcggggcagcagccagccatggtcacgtcgacgatgtggtgaccggatatagagatggtgatggctttctgctctgcaaaatttttgaagtcggcggcggtgtgggacgcccctctatggaccacggagcttccgaggtcggtgccggtatgggacgctacctacttccggcatcttctccggtggtatgggacagtgactgcttcggcgataaatattttggtaaagttgttgcttgtttgctcccgcgagcagccgagggagagaccatagccaccacctctacctctcctcccgccccacttcccctcaccgcccggagggtgccactaaggctggacatgggccagctcgggctgggctcggtccgagccgcagttttttttagaaaaggaggatgacccccggcctctgcatatgggcgatgcatatggccactttattaataattttcacaagaccttacaaagtcatacaacagtaagactaaagccgccgtctaagcaacaaactgtcgctacacctatccagttaagaaatgggcgcagatagcctgggcctaataccaaagagacatcgtatccaagcctaacatctaagacccgagaccccaacctagccacttgccgggtctggggcatacactggtcctgcgtgctctcagaggccgccgccaactggcaccgctccatcttcagaactgtactgatgcatcaaccttgcttggtctagctatcatcgacgccaccacggtgcccaacgacacctcctccctgcgcgcaaacagctgagcacgtcgcggccgccactgatacacctcagctccatgctgccaagtaccaccaaccgacacagcttgaagtccttgaaagatcaatcatgtgtagcacctgccgaccaggcatgacaaagcgtagcacctgtaggtcgggcatgacttgacatctccatcgaagctccgtgcacctgttcgagccgcagttttgctcgctgagaaacaactcggctcgggctggcttgcttgacccatgggctggaaaatcaagctcggctcgaactcgctctggctcggtcaaactcgagccggctcgcgagccctgaaggaaacagtcactagtcactgacaatgggacccaccttatcttcctctctaagctcatgcctgtaggtcatgggacacacgatttcgcccaaattccccagccctacagcagcgcaccgcacctgcagcagcctgccgcccaaattccgtcgtgaccaaggctggacacaagcgcaccgccaaaattctgctagttgggagagaaagatcgagcgagcgagcaccgcacctgcagcagcctgcgccgtcctctcacgagggctctgcagccgcaagcatcacgccggccccgcctctgtggttgctgccgccgctcgtacgggccatgccgaagccgcagagatgaacgaggggtcctccgctgcagagatgaacgagggatacagagatagatcgagcgagggagaccagagatggagtgtgtgggctcgcctgtgcgcatgcgtgcatgaggccacaaattcttcacgggccaaatgggctggaccgagccaaaagctcgccgcggtccaagattttgggctcgactctgtccacgcctatctcaggccgagcccagctcgggccgagccaagatctcgcccccgagcgtccaggcttaaggtgccaccggggaaagcccgggtggcgccagggccatgttcctctccctcgcaaggcgcgcggcagcgctggctgctgggcttcgtggagggggctgcgcgcgtcactgacaatggctcgctgcggcgtcttggtggtggctgctgcgtggcaggcttcgtcatatggatggcgtagcatagcgtggcggcagcaggagatcaggaacaggggcggtgaccctattttatcgggggcagcagccagccatggtcatgtggatgaagtggcgaccggatatagaaatggtgatggctttcttctccgtgaaatttccgaagtcggtggcggtatgggacgccccctctatggaccgcagagcttccgaggtcggtgacggtatgggacgctacctgcttccagcatcttctccagcggtatccgcctagtgagatgtcctcagccgacagagggattgatgacagtgattgcttcggcgataagtatgcgcgcactctggtgaaaacacaagaactttgaccaggcaatgtcgatgcgtctacctcgtgcccttgatgaaggtggtggctcgaagctcagccttggatatgaaagtcctgagatcgacctacggttggactcgtcatcatcagcgcacatgcgatgaacccttcttgaagggcttgactaggagttgtgtactttttgttgtttcggttcttgtcaaagggttcgtggtaagtttgtggagatactgttgagaggtagacatcctcgttttttcgttttttattctacttgtttggcatcgctgcctggttttgcctgccttttttgggtaatggataatatatagctgtgtgcatcaaccgatgttgaggccaggggttttcctcctttacgaaagaaatttgtgccttagataagttctcgactcacaagaatatgtcacttaacaaatcttttttttttgtctacag

SEQ ID NO: 8 - DNA, 6523 bp, Aegilops longissima
  source 1..6523: mol_type = other DNA
ttagtttctaacactaactctattgtttatgttcggtcattgatgccttatctattagctggttatgatattttttattttgatagggaatctaatcatatactaccacagctaggatcctggtttgcgtgtattataatctaccctcttttattattagtttcccaagtgttcttttcatataattgacatccctagacgttaatttcacacaaccaccatatcatcatggataaagagacataccaaatgaattcgtgtaaaatatataagcccgaacggaagtttgaagaaacattcactgatgaaacaacactggaaagaaaaaaaattggatggctcctgacccacaccgtcctctggacctctcgacccataaaccctcccgccggtgacggaccaccaagccgcgccgacagatcaatccttccaccccctccaccctccattctccatgaaatcgctctctactactttgacgatgaattgccttcccatgcttccctgcaggctttcctcggggtcggcaacgccctgggcatcgccttcgaggaaacggcctggcttggcaaaagcttgccaatctccatttgtagcccatcggaacccacttttctgatgttttcctccgtccacaactccatcatcatttttgatgagctgcttatggcagttctgatccatctgtcattgggggagtttccgcttctatgtcgtctcacccaaaatcatgaaaattgttttatcccaagcatcgatctccaccaaatccttcaactagctcttcaaccatgtttgcgtggctccttctgccacgaccattcaaccgcctctctctacctttggctctctgggcggtgcaaagcttgcacccaccaattcagggggtaagatgctctctgatgtgcctcatgatccacagccggcttcgccatcaccaaacttttactacaagttcggtgggttgtctactttggccgatgggccttcggctcctcaaagatctgcttcaagttagcacaaccaaatccagtctaccttagcgtgagatatgtgacgttggtaatgttaagttcttgggtgcccaggtttaagctttcggttctttattttacagaattaccactcagaggtagttttttctctgtttgctaacaaacattcaattaaatctacttgcatttggattagcttcattttagcaaagggaaaacctaatgcgtttatcgtaaaaaaacactttagaccaccttttctctaggcttaatgctttcaatgtctcttgcgtccatgattatatatgttttcctcacctacatgtcctcttctagtatcagcactgaaattgtcaaaaatctcctctaaaactacctgggttaaccttccacctctccatttgccctgaacctacacgccaaattgttatttcaaacactgcactcaatcttgttctctctcctctccaagcccagcccaaattaagatccctccaacgtgcccatcttcataatgggcctgttgcacgacacgtgcctttggcaaaagtttttgcatgtgcgtgttcatcgtgatttgatgtgatcgatcctgtgggggaattccaacgattggtatcagagcaaggtcgaggtgacgttgcgcttgccccgaggtggcgacccgactagcacctcggggtgggcaatgaagtcactaggtgatgcgggcgatatggtcgctggatagtgcagcgacgaggaggatcgggcgatcgtcgttcggcggagcgacaaggagggggacggcaggctgtcgtcggcaaggcggaggaaggagatcgttgacgggagtggtggtgccgaggtgcggtaccggaagagtcgtcaagatcgtcgtccgcggggatagatgagttgaagagtcgtgcgagtttgcgtgactggttgtcgtcgtgtcagtgagtcgtcgtcatgtccaagtgcttgtctctctgaggattgttgaagatgaagcgtgtcatgttcgatgggctgtctggtgtggtgtagcaccaagggcgaagacgtgaagtggagatcaagctcaggcgtatagtgggacgaagaccagcgaaggggagcctcttcaatgaggacctgtctctacatgggacgatgtgtgatccgttggcatgcattgcgttaggggtgaccggtggtgttgcctggcagcgtgactgagagtctgaatcatacgttgaggggatcgtcaagttgcggatctacaacaagtcaaaatcaggagaagtgcgaggaagatgacaagtcaagattagaggaagattgttagcataatcttgattatagattaattagttagtgctagttagatgctactttgctggctgccgtgtagtagtccgtattggagtcaacattgacgtacgtgtgggttgtgtactagatcgtgtccggcttgtaattagcttgcatgccgagtcgtcatagtctgtatgagcgttggagtctagtcctgttaggattgttgtcatcgtgtgatagtaggtttagatcatggtcggttgggcatgtatatatatacagccctgcgtgagggttttgtaacgacacccaaaaaacaagaaaaggaaataaaagaaaagcggcacgacacgtgcctttgacaaaaaattttgcgtgtgcgtgttcatcgtgatttgatgtgatcaatcatgtggggaattccaacagggccaaccgcctgcatgccagccacctgcctatgagtagaccaaaccaacgtggctaactacgaccatcgaccgaggcaggcgatacgaattcccatattttccacgttcaaaacacgcaaacagcacttccctctcgccatctgccatctcgatgggtcaaaaaggggatgtgaaattaagcaatctcctccacttcaatggagaagatcaatgaactacccccctgcatgtgctccacgagcacgaaaaaatcaacgaaacttgtaaccctacggccagcgctgtctagtgctcgtcgcccggaggggcagaacagatgccgcagtcgccgggccaaatgtccagtcacgatatttctgcctcaccatatcgatatgctctgctcaactctgtaaatggtaaatctcagtgaagatccagctctttctacggcgtcctcctccgcaaaaagcgaagtgcaccatatctaactcctggccatgcccctttacctcgctgtttccgcaattatttttatggtgtagaagttcctgaaatcatgaaagaaatactggaacactatgaccatgatgtaagatggtctcatggtgggattgcacaaaacatgactttatgcgccctcaacgggaataaaaacagtgatacatccatatatgtgagagtgagctgaatttttttcccggggcacctaaattcgtgttttcgcgatcactctgttggatctttttatgttattaccaaaaaaggcttttgcaccgctttatatataaagcaaagatcaacatcatccgatacaaacgcacgcgaacaccacacacacccaaggcaggatacaaaagtgctgagcacagcaacaccacctctagcacacaccacgaagaattgaagccgcatacaacgaaccgtggactccaaggcggtgccttcaggaaggatacgacaccggagcgccgccactagggctggacacgagccgagtcgagccgagttcggtccgagcctggctttggctcgccatgaacgagccatgctcggctcggctcgctggttgaacgagcccgcatctggaggctcggctcgtcttgcctatggctcggtccagcccgagcagctcgttttgcctctggaggctgggtgcgtgggcgtacgggcgcatcaggcgaggctggagcttgacggcgttgaggggagcacgacggagacgtgccggcgtggcgaggtcgggcgtctggtgcgtcgaacgcaggtggctagcctccgtagcagttgatcttgcttcctctcgtacgtgtgcatccagccaactccaggactcaatcaacgtgcttcaagttttgtacttgtgtccaagaatcaatcaacaggctggtgatgcgtgcgtgcgctgcggccgctggatgcgatcgtgcgtgccgacgccggcggcgggatgcgtcgtggtggcatgcaggaaatagcttggcgagaagcgtggggcggcggcggcggcgaaccctagcgggaggagacagtggagacgacgcgtctcgctgggttgggctggttgcgctcctctactgggctgcgtgattgcgtcgcgtcctgggcttcggcttcggacgagccaccgagctggaccgggccaaaacaggctcggctcggtcggccaacgggcccatttcatgtggctcggctcggcctatttagctctcgggccgagctgagtcgggccgagctggagcgagctccgagccgagcccattagcccgttcggacgtccagccctagccgccaccgcccgatccgaggatcagagtttcccccggagccgcacgacgggcggtgagagccgcgacgacgccttcaagaagggaacgagcttcgccatcgccggtccgtccgaagatagagcaggttttcatcccgaccaacactcaccgtcgccgaatgctacaccccggcaaccacgccgcccacatggccatggtcaccaggcagctccaagacacgggctctgcccaagagcaccgtgcactgccaccagggccgccgccgaggcatccaagaccttgacaccaccgcacccgagactcgccgctaccccaaccaaatagacgagcagaaaaggatccgcctttgcacccctggcccaccctcagcatcgagacccaataggtcggccagcactggcatccatcgacccatcctgcagcccgaagcgtgagacgagatcggtcctgcagtaccgtggggcggggacgaggtcagtcctgctgcaccgtgcgcgagacgagcttggtcctgcggcactgtgagcgagacgagctcggtcctgttgcaccgggtgcaagacgagcgatggaccgcagctgggaaagggccagcccttcgaagggagtagcgatcgagcgtcgaggagatggagtgaaggccaaaacggtctgatcgcacacaagccgacgccggagaagctggccgccgccgcaagcggatccgccaagccaacatggagccgccacgacacctggagtccaccaccgccggacctccacacatcgccgcccacggccggagcaacgtccacgcccgaccgctgccctacccgcgtctcccggcaagctcatagcgccggagccgtcgggcatgcaccaccgccaccacgcgccaccggccgacccccaccaccagatccggctggatccagcaggggcggaccgcgcgccacctcctgagacgggagcgccgcagccgccccgccacgcacgagacccagggcaccggcgcgccgcgcgagcccgccgccgagttgctgaaccccatgaagtccagtaccacagctcgaggaggccaccctgcgccggcgtcccacgagcagggagggagaagggccccgccgctgccgcgcccaccgggctttgaccgacggagctcgctggcgacggcggggggggggggggggggggggagggggggaggggtgaggggaggaaggggggtccgaggctgcagcaggtgaggttcccccctcccccagcgcgcgcgtgcgcgccgtgggggggggggagggggtagggggaggagagaggggaggcgaggggcagcggggcgagcctggaggcgctcgccctgcggccggcggcggcgggatcaggagagaaggccggcggcggcgggaggggaaccctagcgggggagcgggggagagccttcctttttatgttattgtgatgatgttccagtatctttttctcataattattgagaaattttaacccgttgaaaccttaatgggttcccagacactaggattattgcaatttaatgaatgaattactgtggaaattaaattatcatatatccacgttttatttatgtaaaagtagtactattatgtatccaaaatttaagttcttttagatcggtaccaatcaaacaattctgatttcgaaaacatgaaactgatatcattacatgtcatgaactatatattttagtaatctataattataattatgtgaggcaacatgtttttgcatagaataaaatggtcagagcactatctctaagatcgtgtcaatgtccaaaacagcttacattttgaatgataggtactatattctctgaacatattattgcatattaacatctaatcgattgttgcataacgattaatcacatggcaactatacgctctaaataaccattatatatatgaaaatggcatagtaggggcattcctactgtattttaaaaaaccaaatattatgcgctaattctgtccaatgtgttttgctttcgttctacaacacgttttgacagtagttacttctacag

SEQ ID NO: 9 - DNA, 117 bp, Aegilops longissima
  source 1..117: mol_type = other DNA
gtacggaactccgaagcccagtaattttcgttcttcttagctactgtctgcaaaactctagcacttaggggttattgacaagaagggtaacaagaaacaaatcttgttcttctgcag

SEQ ID NO: 10 - DNA, 294 bp, Aegilops longissima
  source 1..294: mol_type = other DNA
atgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaag

SEQ ID NO: 11 - DNA, 1156 bp, Aegilops longissima
  source 1..1156: mol_type = other DNA
ctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcgg

SEQ ID NO: 12 - DNA, 3001 bp, Aegilops longissima
  source 1..3001: mol_type = other DNA
gctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagag

SEQ ID NO: 13 - DNA, 83 bp, Aegilops longissima
  source 1..83: mol_type = other DNA
aattgtggtcgacttccggagatgctgaagcatcgacttccggagcatcgactgctgaagaagctggaccatcgacttcttga

SEQ ID NO: 14 - DNA, 22 bp, synthetic construct
  source 1..22: mol_type = other DNA
cagctcatctcccttcaagaat

SEQ ID NO: 15 - DNA, 23 bp, synthetic construct
  source 1..23: mol_type = other DNA
gtttacccgccaatatatcctgt

SEQ ID NO: 16 - DNA, 25 bp, synthetic construct
  source 1..25: mol_type = other DNA
tttaaaggcgaggtggataaaatgg

SEQ ID NO: 17 - DNA, 27 bp, synthetic construct
  source 1..27: mol_type = other DNA
gcttgattgttccccgaaaagaatacc

SEQ ID NO: 18 - DNA, 45 bp, synthetic construct
  source 1..45: mol_type = other DNA
cccttgacccgaatctcccgcgcgcgtgtccggcctgcaattcac

SEQ ID NO: 19 - DNA, 21 bp, synthetic construct
  source 1..21: mol_type = other DNA
acggcactcgcgatcatctcc

SEQ ID NO: 20 - DNA, 33 bp, synthetic construct
  source 1..33: mol_type = other DNA
gcgccgtctcgctcgatgagcggggttggggag

SEQ ID NO: 21 - DNA, 30 bp, synthetic construct
  source 1..30: mol_type = other DNA
gcgccgtctcgtgtcaggccaggaccgagc

SEQ ID NO: 22 - DNA, 35 bp, synthetic construct
  source 1..35: mol_type = other DNA
gcgccgtctcagacagtgattgcttcggcgataag

SEQ ID NO: 23 - DNA, 37 bp, synthetic construct
  source 1..37: mol_type = other DNA
gcgccgtctcgatctctcaaacccaaatcaaccatcc

SEQ ID NO: 24 - DNA, 45 bp, synthetic construct
  source 1..45: mol_type = other DNA
gcgccgtctcgagatgaagatcaaaaagacacattagagtctctc

SEQ ID NO: 25 - DNA, 35 bp, synthetic construct
  source 1..35: mol_type = other DNA
gcgccgtctcggaagcatgggaaggcaattcatcg

SEQ ID NO: 26 - DNA, 44 bp, synthetic construct
  source 1..44: mol_type = other DNA
gcgccgtctcgcttctgtggaaattaaattatcatatatccacg

SEQ ID NO: 27 - DNA, 36 bp, synthetic construct
  source 1..36: mol_type = other DNA
gcgccgtctcgctcgtcaagaagtcgatggtccagc

SEQ ID NO: 28 - DNA, 18 bp, synthetic construct
  source 1..18: mol_type = other DNA
atgagcggggttggggag

SEQ ID NO: 29 - DNA, 21 bp, synthetic construct
  source 1..21: mol_type = other DNA
tcaagaagtcgatggtccagc

SEQ ID NO: 30 - DNA, 24 bp, synthetic construct
  source 1..24: mol_type = other DNA
agaagctggaccatcgacttcttg

SEQ ID NO: 31 - DNA, 46 bp, synthetic construct
  source 1..46: mol_type = other DNA
tttaaacggatcacttcgtgcgtcgacggctgtggactatctggac

SEQ ID NO: 32 - DNA, 17 bp, synthetic construct
  source 1..17: mol_type = other DNA
atgagcccagaacgacg

SEQ ID NO: 33 - DNA, 18 bp, synthetic construct
  source 1..18: mol_type = other DNA
tcaaatctcggtgacggg

SEQ ID NO: 34 - DNA, 22 bp, synthetic construct
  source 1..22: mol_type = other DNA
ggaagtatttccccgaacaagc

SEQ ID NO: 35 - DNA, 20 bp, synthetic construct
  source 1..20: mol_type = other DNA
ggctcacatcacaacgaagg

SEQ ID NO: 36 - DNA, 18 bp, synthetic construct
  source 1..18: mol_type = other DNA
taccggcaggctgaagtc

SEQ ID NO: 37 - DNA, 18 bp, synthetic construct
  source 1..18: mol_type = other DNA
ttcaagcacgggaactgg

SEQ ID NO: 38 - DNA, 19 bp, synthetic construct
  source 1..19: mol_type = other DNA
accttcgcgcaatactcag

SEQ ID NO: 39 - DNA, 20 bp, synthetic construct
  source 1..20: mol_type = other DNA
tgttgagaaccacctcctcc

SEQ ID NO: 40 - DNA, 22 bp, synthetic construct
  source 1..22: mol_type = other DNA
agctgccagaaacccacgtcat

SEQ ID NO: 41 - DNA, 24 bp, synthetic construct
  source 1..24: mol_type = other DNA
acttcattgtaccagccgccaact

SEQ ID NO: 42 - DNA, 30 bp, synthetic construct
  source 1..30: mol_type = other DNA
taatcaaactcctcgaagatgagaccacta

SEQ ID NO: 43 - DNA, 29 bp, synthetic construct
  source 1..29: mol_type = other DNA
ccagcaggtcctcaacgtcgtgggagatg

SEQ ID NO: 44 - DNA, 26 bp, synthetic construct
  source 1..26: mol_type = other DNA
gagataagctggacaagctgaaggac

SEQ ID NO: 45 - DNA, 26 bp, synthetic construct
  source 1..26: mol_type = other DNA
ctgccatgctgcgatggacttattcc

SEQ ID NO: 46 - DNA, 29 bp, synthetic construct
  source 1..29: mol_type = other DNA
aaaattaataaatttgatattgcgaagcc

SEQ ID NO: 47 - DNA, 29 bp, synthetic construct
  source 1..29: mol_type = other DNA
ctttgatgtcgaagatgttgaatttctca

SEQ ID NO: 48 - DNA, 28 bp, synthetic construct
  source 1..28: mol_type = other DNA
tggttgatttgggtttgagagacgaaga

SEQ ID NO: 49 - DNA, 27 bp, synthetic construct
  source 1..27: mol_type = other DNA
acatcccaagaaggtagtcgatgcagt

SEQ ID NO: 50 - DNA, 30 bp, synthetic construct
  source 1..30: mol_type = other DNA
ggattataattactacacgtaaccaaagga

SEQ ID NO: 51 - DNA, 27 bp, synthetic construct
  source 1..27: mol_type = other DNA
ctctaatgtgtctttttgatcttcgtc

SEQ ID NO: 52 - DNA, 29 bp, synthetic construct
  source 1..29: mol_type = other DNA
tttcctcaacttatcagggtgttccgtcc

SEQ ID NO: 53 - DNA, 30 bp, synthetic construct
  source 1..30: mol_type = other DNA
ttttgagcaaccggacaaatttaggaatga

SEQ ID NO: 54 - DNA, 33 bp, synthetic construct
  source 1..33: mol_type = other DNA
tcctgataagtttggtagcctaccaaatatctc

SEQ ID NO: 55 - DNA, 35 bp, synthetic construct
  source 1..35: mol_type = other DNA
ggtatttcaaatgaataagctggcaaaatgattcc

SEQ ID NO: 56 - DNA, 28 bp, synthetic construct
  source 1..28: mol_type = other DNA
ttgccatggcctcagggaactcccagag

SEQ ID NO: 57 - DNA, 29 bp, synthetic construct
  source 1..29: mol_type = other DNA
aattgagatgcctcagcttaaacaagcta

SEQ ID NO: 58 - DNA, 30 bp, synthetic construct
  source 1..30: mol_type = other DNA
ttgttaaccttgagtctttggagcatttcg

SEQ ID NO: 59 - DNA, 30 bp, synthetic construct
  source 1..30: mol_type = other DNA
tgcagcttggaacaacttattaggttcaat

SEQ ID NO: 60 - DNA, 24 bp, synthetic construct
  source 1..24: mol_type = other DNA
agttgccacgagctaaaagacctc

SEQ ID NO: 61 - DNA, 30 bp, synthetic construct
  source 1..30: mol_type = other DNA
gaggatgggagcttttcaagcctcatacag

SEQ ID NO: 62 - protein, 921 aa, Triticum sp.
MAPCLVSASTGAMGSLLTKLETMLDDEYILLNVRRDIKFVIHELAMWQSFLLDVADTEEPGQHDKSCADLVRELSYDIEDKIDNSMSLMLHHACPKSGIKKHMSKFKNLLPVKIPYQIAKDIKDIKSQILEVSNRCERYRFEDVCLARTEFVDPRLCTVDTCAADLVGIDGPKHELVKWLRNGEDESVHQQKVVSIVGCAGLGKTTLAKQVYDELRINFEYRAFVSISRSPNMATILKCVLSQFHAQDYSSDESEIPKLVDQIRDLLQDKRYFVIIDDIWDMKTWDVLKCALCKNSCGSVIMTTTRIYDVAKSCCSSNGDLVYNIQPLSVADSEELFLNRVFGHEKGFPPELKEVSKDVLRKCGGLPLAINAISSLLAAEKIEEWDRVGLSNVFAQGEKSDIDAMKYKLSLCYFDLPLHLRSCLLYLIMFPEDCLIEKERLVHRWISEGFIRNEDGEDLVEVGERYLYELVNRSLIESVGVPYDGKARFYRVHNVILDFLMIKSMEENFCTLTSNQSRLDYKVRRLSLFANKDPSCIAQLDLSHARSLGASGHLGQLISSVKSNALRVLDVQDCSELGNHHVKDIGRNPLLRYLNISGTDVTELPIQIGDMGFLETLDASFTELVEMPGSITRLRQLQRLFVSDETKLPDEIGNMKRLQELGDINAFKQSVNFLNELGKLTGLRKLGIIWDTNDILKSGKGSSKEKRLVSSLSKLDAGRLSNLYVTFYLREKDGFIGHPFLPALNSIREVYLRRGRMCWMNKWLLSLANLEKLYISGGDEIEQDDLRTVGSIPTLVEFKLYSGCLGPIIISSGFEQLERLELKFSFSQLTFEVGAMPNLKKLDLHVYLSKFKSAGAGFDFGIQHLSSLACVSIVIFCEGVSAAYVEAAEGAFKSMVNAHPNPNRPMLEMTRESADFMSQDE

SEQ ID NO: 63 - protein, 1080 aa, Triticum sp.
MATAWDVASVGWSMVVLGWLVSPVITLILPKILSRLGFDAAQKLQGLVISIMPEMENTLRAVDQERMMLRGKKSNSDVAALDKMAAMLRHAREDAEDIFDDAHEKICVGDFLYGVVQSFLDGIARIIRSRWAWLMQWARRSSLCRFLEHASGEAVTIGVAASNEPGHVTINVAAPSGGESIPVTTSAAASHEPTAPSSDSLGRCLSCFSSSFNFVKYYCTSSFNWLRFYRDWSYEVVGVTTYQENATALDVVLTAISRWNLNKRIEKVQSTISEVTKSPLLGTASKSAPDDIANKNRSRIRTASKRKVFGREGLRDHIMVRLREIPEHDAASSSADPCYSVIGIYGVAGSGKTTFAGYIQDYIKEECKDEKLFDTIMCIHVTETFSVDDIFHEMLKYITGDSHSNISDRGALDKKLKEALCGKRFFLILDDLWVKNKNDQHLEELISPLNVGLKGSKILVTARTKEAAGALGADKFIEMPDLDEDQYLAMFMHYALSDTRVALQEFEQVGREIAKKLHRSPIAAVTVAGRLGANPNISFWKNVAKLDMLNYTMDALWWSYQQLDPDIRRCFEFCSIFPRKFKLEKDQLVRLWTAQGFVKTSCATEEMEDVAEGYIQELVSCSFLQEKGTGSGVDHFTIHDLLHDLLVKVAGNDCFRIENRWSKQGECQEDVPRDVRHLFVQKYDGELITRKILGLENLRTLIICVVEENRPVEEKVIESMCVRLLKLRVLAIAFNKKCRSTSNPSDKFLVPESITQLKHLRYLAFRKSDNCKVILPSALAKLLHIQLLDFGLGKISDFTCAADLINLRHIFCWDVSFPNIGTLSSLQRIPFFRLRNEQGYEIKQLRDLNKIRGMLVVNGFENVKSKEEALEANLAAKERLTELILCFLTSHTRGNGNAEVAAEVLEGLCPPVGLEALLIYDYNGSRYPDWMVGKLTGGPKNLKLLEIYGCSRLGPGPQLEAFPHLRMLNLEDCSWDALPGNLEHLTSLKALKIEHCMNIRSLPTLPQSLKLFILRCCDVELMKSCETEGDPNWHKIKHIPDKMVSGQPHSIYHIRRMFRVSAPVPTLSTSGDGDTTDERR

SEQ ID NO: 64 - protein, 912 aa, Aegilops tauschii
MAEAALLLVTTKIGKAVATETLHYARPWLTKKAGSIAELPTNMTLIKNDLEVIQAFIKDTGGKGLIDGVTETWIGQVRRLAYDMEDIVDQYMYVVGKHHQKGSKWSYVKKIVKKPQYLFTLDQIATALERINQALIRLKENKDWTQPIAGVGDVYATNYDSQQQLYLPGHDYSISDDELVGIDKNRDILMGSLNLEKSLDLQTIALWGMGGIGKSTLVNNVFRNEASNFECRVWVSVSQSYKLDDIWRIMLKEIYSKDQKAFDGEKLTCAELQDELKETLKTKRYLIILDDVWTAEAFRKIKGVLVDTKMGSRIIITTRFDEVASQADDGYKIKVEPLEKEDAWRLFCRKAFPRTENHICPLALRKCGESIVEKCDGLPLALVSIGSILSLKEQNDTEWGLFEAQLISELNNNSDLKHVVKILNLSYKYLPDDLKSCFLYCAMFPEDHMIHRKRLIRLWVAEGFIKQNGNCSLEDVAEGYLRELVRRSMLHVVERNSFNRIKCVRMHDLVRELAIFQSKRESFGTTYDDSHGVMQVDSRRMSVLQCKNDTQPSVGQCRLRTFIAFNTSMGSFPWFSSESKYLAVLELSGLPIETVPNSIGELFNLRYLGLNETKVKVLPKSVVNLHNLETLSLRCADCLNLPRGSKKLKSLRHIIMVKLLDHTWLSFKSFESMEPFDGLWSFKNLQTLGAVRASKAFVAKLASLSQLRTLSISGVRGIHCAQLCDSLSKIDQLSALEITASDEDEVLQLETLTLSNGLRKLCLYGRFSEGTFKSPFFSSNGDVLHEISLRWSQLSENPVPRLSELSNLTAIVLSKAYTGQEINFQPVWFPNVKSLSLWDLPHVNQICIHEGALVRLEELVIRNLAELRDIPGLGHLKSLKDTQFIDMHPDFVSNLQAEKLEHIPISYYRTVR

SEQ ID NO: 65 - DNA, 2358 bp, Aegilops longissima
  source 1..2358: mol_type = other DNA
ggtaagtttgtggagatactgttgagaggtagacatcctcgttttttcgttttttattctacttgtttggcatcgctgcctggttttgcctgccttttttgggtaatggataatatatagctgtgtgcatcaaccgatgttgaggccaggggttttcctcctttacgaaagaaatttgtgccttagataagttctcgactcacaagaatatgtcacttaacaaatcttttttttttgtctacagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggttagtttctaacactaactctattgtttatgttcggtcattgatgccttatctattagctggttatgatattttttattttgatagggaatctaatcatatactaccacagctaggatcctggtttgcgtgtattataatctaccctcttttattattagtttcccaagtgttcttttcatataattgacatccctagacgttaatttcacacaaccaccatatcatcatggataaagagacataccaaatgaattcgtgtaaaatatataagcccgaacggaagtttgaagaaacattcactgatgaaacaacactggaaagaaaaaaaattggatggctcctgacccacaccgtcctctggacctctcgacccataaaccctcccgccggtgacggaccaccaagccgcgccgacagatcaatccttccaccccctccaccctccattctccatgaaatcgctctctactactttgacgatgaattgccttcccatgcttccctgcaggctttcctcggggtcggcaacgccctgggcatcgccttcgaggaaacggcctggcttggcaaaagcttgccaatctccatttgtagcccatcggaacccacttttctgatgttttcctccgtccacaactccatcatcatttttgatgagctgcttatggcagttctgatccatctgtcattgggggagtttccgcttctatgtcgtctcacccaaaatcatgaaaattgttttatcccaagcatcgatctccaccaaatccttcaactagctcttcaaccatgtttgcgtggctccttctgccacgaccattcaaccgcctctctctacctttggctctctgggcggtgcaaagcttgcacccaccaattcagggggtaagatgctctctgatgtgcctcatgatccacagccggcttcgccatcaccaaacttttactacaagttc

SEQ ID NO: 66 - DNA, 4435 bp, Aegilops longissima
  source 1..4435: mol_type = other DNA
ataattatgtgaggcaacatgtttttgcatagaataaaatggtcagagcactatctctaagatcgtgtcaatgtccaaaacagcttacattttgaatgataggtactatattctctgaacatattattgcatattaacatctaatcgattgttgcataacgattaatcacatggcaactatacgctctaaataaccattatatatatgaaaatggcatagtaggggcattcctactgtattttaaaaaaccaaatattatgcgctaattctgtccaatgtgttttgctttcgttctacaacacgttttgacagtagttacttctacaggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagaggtacggaactccgaagcccagtaattttcgttcttcttagctactgtctgcaaaactctagcacttaggggttattgacaagaagggtaacaagaaacaaatcttgttcttctgcagaattgtggtcgacttccggagatgctgaagcatcgacttccggagcatcgactgctgaagaagctggaccatcgacttcttgagacccaatgagaggaagctagctggaagtgatcctgccatatacagagactaaagcaaattaagctgaaatgcaagaggtaataatgagaaacatgtaatgtttttcttcaacgtaactggacagtgatttcatcgtaattggttatgctggtgtattctgacaatctggaactctatgtgttaagctttgtcctgaacatatctggtctttcttttcagtttagagctagtgcagcactacctattgggctggaccttcatgccaatccaagctttattggtggcagagtcggaggcggcagcggcgctatgccccacggaagggatcctgcccggaagatcactcccgatgcattctgatcctaccaacagcggcgatggtggtgcatgcccatccttgtgagctccagttgagctgtaagagtctgcttctcccggatctagttcacctgatggtttcctctcatgcatgtgaataaaataccttgtgctagcaacacaaatattccacagagaatgcattgtcctcaaattgcttgcgttgctctggctaatttcgagctgtgtcctaaaatttatctcatctccttcgtttcacatcacactctcctcaaatttcgtcctggaaagagcgaatcaagaatgacgaacaccatgttggttgcacgactgaactgctaactgttctgcatatatatacttggcactgaccggtactagtacatctcgtgccttttcaggatagagacagaccatccagcaagcagtagttttttaacctatgtaccctggtccagtttttagcataagtaaacacttgcaacaacttttacttgcactattattttcggttattatttactccaaaatacaaaaaacatgtatcacaatatcgtaaaaactca

SEQ ID NO: 67 - DNA, 11734 bp, synthetic construct
  source 1..11734: mol_type = other DNA
tgtccggcctgcaattcacgttttcgtatgcctcaaaatcaaagttatcggggttatgttttaacgaccccccccccctcatgttgaagttatcggggttatgttttaacggccccctctctcatgttttcatacgcctggaaatcaaagttatcggggttatgttttaacaacaagatgaatacaagatttatacacaagataaatacaagatttatacacatggatgaatacacaactcaacaaatgaaccaaccatcatcctatgaacaaaactcaacatgcaacaaatggacaccttttgtcgaacaacggagggatgatcagggaccaactccgcggagatgcaacaccacaacctgtaattccaccgattgaaaaaaataggaaaaccccaagtgtagtagcgcttgcatggcaagaaaacctagtaaaacctagtaaaaagaagtatatttgtcaccaaaatacaacacttgacatggcgaaaaaacaatttttttataaaggagaaaatagatcctgttgaacttgagcatttctcggactttgaaatgctcctgttgaactaaaactatttcaaatgaactagacagaaattttccatattacttctaccatgccaaaaatggttctccctaggccaaaaacttgacagaaacttgacaaaaattgcattagaacttgtccatgatctcatcgatcttgagcagagtagagtatattgtagtagagtataactaagaaaaaattcaccattcttaatatatgtttgtgacatcactaaagaagatttgaagcaccatcgaacaaactaatgaacatatctcctaataatacttgtatgctgcacaaaacaatcactttctacaaaagacttttttgtgagcaacatcaaagtagagaacatgattaattatagcctttttccttttgcaatttgtcatgccattaagacctatgcaacaaaagaccacatgcttgataacaaagccatgaatacaaattgaagtcagttaaaagaaccttttagcagaacgattattttaattatggacatcagctagtgaaaagacttgcccttccaggatttaacctttattcagtaggtttttaacacaatgaaaacaaatagttgatagtggacttgtggatacatgagcattcaaaattttcagaccacaagaattaggagtgctttaagttcatacattgcccaaaaaaatgagtactttcagataaaacagtgtgtagaaaacatagaacattcaatgtacactttagttatgccagtgtaaaaaattcagaacattcgattatgagagtgtcaataattgagaagtcagtgtgcatacatatcagatcattcttgtaacaaaactgcaacataaattcagttacgaaggtgtaaaagatgcagaacattcagtgaaagacggtcacaactacagcaaattcagttaacacattcaaatatatcattcacttaacaagttcacagaaatcatggacatatctaattcaatttactggaacgaatgagttcaacacttcatatgatcaacagggtacaatcaaacaccacatccacgaccaaatttgacaactttcaactaaaaaccatgaacacattagcaacatcgcaaactcctcttaactactggcactgaccactgaccacttgaccacattagtacctaaggcaatgagatgacaacacactgctccatactaaactatgaagacaaaactttctatgaacaaaaatttcatctaaggccttgtctgttgcatctgagcatatatccagaagatactttgatcatgaaagaaacattaatatggatgtgggtagccgaaggctttgtcagtgagttccaaaaatttctatgaacaaaaaaaacagcaatgcagcggcagcagtggcaagcagagcaagcagcggagcagtggcagcaggggaatgggggagctcactaggggcggcgagcgatggagctggagggcaggacggcggcgaggggagcacgcaggggacggcgtcggggcaggggcgtggcgacggcgtcggggcaggggcgcggcgaggggagcgggggacggcatcggggcagggctcggggaggcgacaacgaggtccggcagcctggcggcatcgaggtgttcatcggcgccgtcgggagagaggaggagattgaagaaactggatggggattgtaattttttctaagtcctgcttatatagccccaccttttagtcctggttggttgctccaaccaggactaaaggcccccctttagtcccagtccgagccaccaaccgggactaaaggtgaattttatcaggcgaaaaggcgggaagcccaggcctttagccctggttggttgctcggaccgggactaaagggggtcctttagtcccggttggaggcgatatgcaagacatgtcgccgaaggagcctcgctagaatcgcgatacgcatgacagcgggcgagatcttttgacacccgaaaccccacatgctcaggaaggaccccatttggacgcacggtggtaatgggctgccctaggtcatttcgcccacccctaggacatcgtagatcgatgctttccaacatgaagaaagtcgaaaaacaagaagcagaaaaactagggcgagagagaaaatgtagaggtttgtagattgttttttcgtttgtgtgtttgttattcaatcggccatcacccctcacgtatgtaagagatagtttgacgttctggacaacgcaagtgcttcgaatcccgtaaaaaaagacccaaaccaagtcaaattcggactgtccggagggtctggatgcccagagggttccggaattatccgggctgtccaatacgggtacagagtaaaacccggatccaggcccggaggctcaggactttcagtcttcgttttggctagctctactcttggatggcaaccttaatgctcatgcatccagcccaaaatctgcctagtactaccagtgtcatatatatttggtcttctgtcgactagggcgtacttccaggagtactaagtatattcccctataattgtattctttaaaatggtacgatgattgagtccaaacagagattggagtgtcgcattagctttttgctggaaacatttcttcctttagaccactcgactagtctgcatcttaattgtattattgattagcacccatcctattcagctatatattggtaaacaagacagagacatgcagtaatttaaaaggactagaaccacgcacagaccaagtcttggtattaacttaaattaaatcgggaggcctggacatgcgtttactttcttcaagtcttcctcaatttgcaaatgtattttgaaactgcattattggctatatggagacaaatcttgggcacgaaccaatcagaccaacggtagccttatctcgtcttccccctcgacgatctcatccactaaccttagagacttggaccgttgatggctgttgcttcgtttggaatcttcttcaatttgcacacaaaccaatcagaccaacggtagccttatctcatcttcctcctctccgatctcatccattagccttggagacttcgaccgctgacggctgttgcttcgtttggaatcttgttaaatttgcacaaaaaccaatcagaccagcagtagccttatcttatctcccctctctccaatctcatacattagccttctgtgtcttggtgatgaccacaagcaagcaaagcgctccttaccttgctgggcttccacaaacacaacaatacccttgcaggacagagcaagagcttgttgtgaagccatgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaaggtacgcatgctagctgccctacgctgtgttgaagcaaaattagttgttcattttttttagatggaagccaaattctttattcgtccactaggcttgttttgggacagagccccttctatcctcatggtcggagcctcttctattctcttatcttcggtgtagtctaccctcagggccggtcctaggattttaagggcccggggcaagattagagctcggggcccattataggattgccgtcagactgatttcctagttcagggtggacggcacgagcggtcttttggaccggctcgtggcccgctcagtccgagctggctcggtcctggcctgacagacagtgattgcttcggcgataagtatgcgcgcactctggtgaaaacacaagaactttgaccaggcaatgtcgatgcgtctacctcgtgcccttgatgaaggtggtggctcgaagctcagccttggatatgaaagtcctgagatcgacctacggttggactcgtcatcatcagcgcacatgcgatgaacccttcttgaagggcttgactaggagttgtgtactttttgttgtttcggttcttgtcaaagggttcgtggtaagtttgtggagatactgttgagaggtagacatcctcgttttttcgttttttattctacttgtttggcatcgctgcctggttttgcctgccttttttgggtaatggataatatatagctgtgtgcatcaaccgatgttgaggccaggggttttcctcctttacgaaagaaatttgtgccttagataagttctcgactcacaagaatatgtcacttaacaaatcttttttttttgtctacagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggttagtttctaacactaactctattgtttatgttcggtcattgatgccttatctattagctggttatgatattttttattttgatagggaatctaatcatatactaccacagctaggatcctggtttgcgtgtattataatctaccctcttttattattagtttcccaagtgttcttttcatataattgacatccctagacgttaatttcacacaaccaccatatcatcatggataaagagacataccaaatgaattcgtgtaaaatatataagcccgaacggaagtttgaagaaacattcactgatgaaacaacactggaaagaaaaaaaattggatggctcctgacccacaccgtcctctggacctctcgacccataaaccctcccgccggtgacggaccaccaagccgcgccgacagatcaatccttccaccccctccaccctccattctccatgaaatcgctctctactactttgacgatgaattgccttcccatgcttctgtggaaattaaattatcatatatccacgttttatttatgtaaaagtagtactattatgtatccaaaatttaagttcttttagatcggtaccaatcaaacaattctgatttcgaaaacatgaaactgatatcattacatgtcatgaactatatattttagtaatctataattataattatgtgaggcaacatgtttttgcatagaataaaatggtcagagcactatctctaagatcgtgtcaatgtccaaaacagcttacattttgaatgataggtactatattctctgaacatattattgcatattaacatctaatcgattgttgcataacgattaatcacatggcaactatacgctctaaataaccattatatatatgaaaatggcatagtaggggcattcctactgtattttaaaaaaccaaatattatgcgctaattctgtccaatgtgttttgctttcgttctacaacacgttttgacagtagttacttctacaggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagaggtacggaactccgaagcccagtaattttcgttcttcttagctactgtctgcaaaactctagcacttaggggttattgacaagaagggtaacaagaaacaaatcttgttcttctgcagaattgtggtcgacttccggagatgctgaagcatcgacttccggagcatcgactgctgaagaagctggaccatcgacttcttgagatccaatgagaggaagctagctggaagtgatcctgccatatacagagactaaagcaaattaagctgaaatgcaagaggtaataatgagaaacatgtaatgtttttcttcaacgtaactggacagtgatttcatcgtaattggttatgctggtgtattctgacaatctggaactctatgtgttaagctttgtcctgaacatatctggtctttcttttcagtttagagctagtgcagcactacctattgggctggaccttcatgccaatccaagctttattggtggcagagtcggaggcggcagcggcgctatgccccacggaagggatcctgcccggaagatcactcccgatgcattctgatcctaccaacagcggcgatggtggtgcatgcccatccttgtgagctccagttgagctgtaagagtctgcttctcccggatctagttcacctgatggtttcctctcatgcatgtgaataaaataccttgtgctagcaacacaaatattccacagagaatgcattgtcctcaaattgcttgcgttgctctggctaatttcgagctgtgtcctaaaatttatctcatctccttcgtttcacatcacactctcctcaaatttcgtcctggaaagagcgaatcaagaatgacgaacaccatgttggttgcacgactgaactgctaactgttctgcatatatatacttggcactgaccggtactagtacatctcgtgccttttcaggatagagacagaccatccagcaagcagtagttttttaacctatgtaccctggtccagtttttagcataagtaaacacttgcaacaacttttacttgcactattattttcggttattatttactccaaaatacaaaaaacatgtatcacaatatcgtaaaaactcaacaacacacttggcacctccacctgttaaaaaaacgcttggcacctccggatactttgtttggtacatcatgtgaattatattttttttttcgaaaagggggaaaacctcggcctctgcatcagaaagatgcatacggccatcttataaaccaaacaaagatagtgtcaacgtggttcgaaggtcttcaaaacagaaaaagtaaaacaagctcacacagagccatagagtggctagaaaccacaactagccaagataagataccacaacgatgattatactagatgcctatcctattacatgaccgccatccaaaccggttgaatatatcccgagctaccatctcccagcggacagatccagtaaccaaatgctccctggactccgtccgagtgagtggcgaccacgaacggatccgtgccgtagttcgaaatataacctgcaaaaagtggataatgatattctgttaaagaccaaatcatttctacaagtccagatagtccacagc

SEQ ID NO: 68 - DNA, 10037 bp, synthetic construct
  source 1..10037: mol_type = other DNA
agtctctatccatagtttctgtaaatattacaacatggcaatatacaaccatgagagatggataagtggaaaacatagacttaatcacaataaggaatcatatgcatatgagcaatggattagtggaaaacatcatgcaaagttgacaacttttataacagccagttccagacgatcgtcgaggactcctcccccctcatgtctgatgagtcgtcctatctttcgagagagcatactttgcggaccgcctctctcatgcttgtccggcctgcaattcacgttttcgtatgcctcaaaatcaaagttatcggggttatgttttaacgaccccccccccctcatgttgaagttatcggggttatgttttaacggccccctctctcatgttttcatatgcctggaaatcaaagttatcggggttatgttttaacaacaagatgaatacaagatttatacacaagataaatacaagatttatacacatggatgaatacacaactcaacaaatgaaccaaccatcatcctatggacaaaactcaacatgcaacaaatggacaccttttgtcgaacaacggagggatgatcagggaccaactccgcggagatgcaacaccacaacctgtaattccaccgattgaaaaaaataggaaaaccccaagtgtagtagcgcttgcatggcaagaaaacctagtaaaacctagtaaaaagaagtatatttgtcaccaaaatacaacacttgacatggcgaaaaagcaatttttttataaaggagaaaatagatcctgttgaacttgagcatttctcggactttgaaatgctcctgttgaactaaaactatttcaaatgaactagacagaaattttccatattacttctaccatgccaaaaatggttctccctaggccaaaagcttgacagaaacttgacaaaaattgcattagaacttgtccatgatctcatcgatcttgagcagagtagagtatattgtagtagagtataactaagaaaaaattcaccattcttaatatgtgtttgtgacatcactaaagaagatttgaagcaccatcgaacaaactaatgaacatatctcctaataatacttgtatgctgcacaaaacaatcactttctacaaaagacttttttgtgagcaacatcaaagtagagaacatgattaattatagcctttttccttttgcaatttgtcatgccattaagacctatgcaacaaaagaccacatgcttgataacaaagccatgaatacaaattgaagtcagttaaaagaaccttttagcagaacgattattttaattatggacatcagctagtgaaaagacttgcccttccaggatttaacctttattcagtaggtttttaacacaatgaaaacaaatagttgatagtggacttgtggatacatgagcattcaaaattttcagaccacaagaattaggagtgctttaagttcatacattgcccaaaaaaatgagtactttcagataaaacagtgtgtagaaaacatagaacattcaatgtacactttagttatgccagtgtaaaaaattcagaacattcgattatgagagtgtcaataattgagaagtcagtgtgcatacatatcagatcattcttgtaacaaaactgcaacataaattcagttacgaaggtgtaaaagatgcagaacattcagtgaaagacggtcacaactacagcaaattcagttaacacattcaaatatatcattcacttaacaagttcacagaaatcatggacatatctaattcaatttactggaacgaatgagttcaacacttcatatgatcaacagggtacaatcaaacaccacatccacgaccaaatttgacaactttcaactaaaaaccatgaacacattagcaacatcgcaaactcctcttaactactggcactgaccactgaccacttgaccacattagtacctaaggcaatgagatgacaacacactgctccatactaaactatgaagacaaaactttctatgaacaaaaatttcatctaaggccttgtctgttgcatctgagcatatatccagaagatactttgatcatgaaagaaacattaatatggatgtgggtagccgaaggctttgtcagtgagttccaaaaatttctatgaacaaaaaaaacagcaatgcagcggcagcagtggcaagcagagcaagcagcggagcagtggcagcaggggaatgggggagctcactaggggcggcgagcgatggagctggagggcaggacggcggcgaggggagcacgcaggggacggcgtcggggcaggggcgtggcgacggcgtcggggcaggggcgcggcgaggggagcgggggacggcatcggggcagggctcggggaggcgacaacgaggtccggcagcctggcggcatcgaggtgttcatcggcgccgtcgggagagaggaggagactgaagaaactggatagggattgtaattttttctaagtcctgcttatatagccccaccttttagccctggttggttgctccaaccaggactaaaggcccccctctagtcccagtccgagccaccaaccgggactaaaggtgaattttatcaggcgaaaaggcgggaagcccaggcctttagccctggttggttgctcggaccgggactaaagggggtcctttagtcccggttggaggcgatatgcaagacatgtcgccgaaggagcctcgctagaatcgcgatacgcatgacagcgggcgagatcttttgacacccgaaaccccacatgctcaggaaggaccccatttggacgcacggtggtaatgggctgccctaggtcatttcgcccacccctaggacatcgtagatcgatgctttccaacatgaagaaagtcgaaaaacaagaagcagaaaaactagggcgagagagaaaatgtagaggtttgtagattgttttttcgtttgtgtgtttgttattcaatcggccatcacccctcacgtatgtaagagatagtttgacgttctggacaacgcaagtgcttcgaatcccgtaaaaaaagacccaaaccaagtcaaattcggactgtccggagggtctggatgcccagagggttccggaattatccgggctgtccaatacgggtacagagtaaaacccggatccaggcccggaggctcaggactttcagtcttcgttttggctagctctactcttggatggcaaccttaatgctcatgcatccagcccaaaatctgcctagtactaccagtgtcatatatatttggtcttctgtcgactagggcgtacttccaggagtactaagtatattcccctataattgtattctttaaaatggtacgatgattgagaccaaacagagattggagtgtcgcattagctttttgctggaaacatttcttcctttagaccactcgactagtctgcatcttaattgtattattgattagcacccatcctattcagctatatattggtaaacaagacagagacatgcagtaatttaaaaggactagaaccacgcacagaccaagtcttggtattaacttaaattaaatcgggaggcctggacatgcgtttactttcttcaagtcttcctcaatttgcaaatgtattttgaaactgcattattggctatatggagacaaatcttgggcacgaaccaatcagaccaacggtagccttatctcgtcttccccctcgacgatctcatccactaaccttagagacttggaccgttgatggctgttgcttcgtttggaatcttcttcaatttgcacacaaaccaatcagaccaacggtagccttatctcatcttcctcctctccgatctcatccattagccttggagacttcgaccgctgacggctgttgcttcgtttggaatcttgttaaatttgcacaaaaaccaatcagaccagcagtagccttatcttatctcccctctctccaatctcatacattagccttctgtgtcttggtgatgaccacaagcaagcaaagcgctccttaccttgctgggcttccacaaacacaacaatacccttgcaggacagagcaagagcttgttgtgaagccatgagcggggttggggagatgatcgcgagtgccgtggtgaagcaaatcactagcaggctaatcaaactcctcgaagatgagaccactatgcagtggaagtttaaaggcgaggtggataaaatggcagagaaaatgaaatatctggcagctgtgatgcatgatgctgatgataaagtgcgccgaggaggaagggttggagaaacagttgggctatggctcctgaaattgaaatccatctcccacgacgttgaggacctgctggacgacttggacactaagaataacgaagcaaagctcaaggtattcttttcggggaacaatcaagcttaccggtggataactaggccccatcgtttgaaggacgtggggaagagaatagaagaaataaaaaaggagggctgcgagttcaatctggatcctaaagcacaagtcgaacaaagacaagaagagacttatgcaaccatcagcgcgacaactggaatgcaagggaggagcgctgaaaaagaaaaattaataaatttgatattgcgaagccaagctaatgagcgtatctccataattccaattgttggccttggtggtatagggaagacaacattggctcggtcagtttgggcagatgagaaattcaacatcttcgacatcaaagcctgggttcatgtgtctgacaagtttgatttgcataaaattgcgagaaccatcatcagaagcgtagataccgatatcaatcttgatagcaatgatttgcattttctgcagaatattcttcgagtcaaacttgctggtaaaaagtacttgatttttttggatgatatctgggaagaagatggagataagctggacaagctgaaggacatgctacagcatggctccaacggtagcaggattataattactacacgtaaccaaaggatagtggaaacaatgagcaccggcattcttgcacatgagagggtaattctcccggtgcatgagtctgacagaatcaaattgggtgttctgccaaccgatgactgctgggaattaatgaagataagggcatttggtcctgatgatgaccagactcctttgaaacaaatcgggtatgaaattgcaagtaaatgtgggggtataccactcgtggcaaatgctttcgggaaagtaatgtcagggaataagtccatcgcagcatggcagtatataaaagataggatggttgatttgggtttgagagacgaagatcaaaaagacacattagagtctctcaagttgagttattacttcatgaagctggagttcaaaatttgtttctcgtatttggcggcatttcccaagggcttcattatagacaccaaccgtttaatccagcaatggaatgcacttggatacatttattcagaacatgatggtaaaaactgcatcgactaccttcttgggatgtcctttcttcagatttcaggaccttcttcggctagtcaaagtccagcgtgtgccaatgcatctggggagcttaccatgcatgatctggtacatgatcttgcgttagtaattactgttgatgaattccttgttttggatgctgatgctactggaccaaggacttggaaaaaagctattcactgccgacatgcagagttgatacaatacaagaatcaatccaatgttttcgagcatctgccaaaaaagcttagatccctccatattagagattcactgaaatggaaactccctccaaaggccttttctcgatgcaagtacatgcgtgtcttggacctagcatgtttagtagggcaactctctgaagggcaatcaacgtcaagcaacatgaggctgccacgttccataaacaaaatgaagctacttaggtaccttgatgccacgggattgccagtttcttcactttctaagtctttgcatatacttcaaaatatggaaactctgataatgtccaattgctcacttgaaaccttgcctgacaatgtttgtagcctccacaaacttagctatcttgatctgtccggtaatagaagccttaataagctacctacgtcgttgggcaagctctctaagctctctttcctcaacttatcagggtgttccgtcctccaaggggtgcctaaatccatttgccagttgacatgtttacaatacctagacatgtcagagtgtcgtgccgttcaaaatcttcctgataagtttggtagcctaccaaatatctcattcctaaatttgtccggttgctcaaaattaaccaaactacctgacattgttaaccttgagtctttggagcatttcgacctatcaagttgccacgagctaaaagacctcccacaagattttggtaatcttcaaaatcttgtgttcttgagcctctctgattgctacaaggtttcagtgctcccggaatcattttgccagcttattcatttgaaatacctagatctttcagattgccatggcctcagggaactcccagagtgttttggtaacctttctgagcttgactcattgaacctaataagttgttccaagctgcaagtactgcctgactcattctgtagcttgtttaagctgaggcatctcaatttgtcatactgtatgaggcttgaaaagctcccatcctcgttaggtgaccttaagcttcggttattggacatctctgctgcaactttccatgatttaccggatagcatccgtggcatgactacactcactaaacttgtggtctcgacagcacaactaggggttcataacaaggttcgtgacattcggcagcatctgaatttgccaggggagatagtacatactgtacatctgactgaaaacaaaggatgtagcagtattgtagagttagcacaattgacctgtcaaaacttgaaagtcttacaacttcagaatgtcaggcatccggaagatgcagagagagccaagttacgtgataaatcagatcttcgggttctatggctttgttggggactccagcgagaagaggatatttatgtgctggagaggcttgtacctccacgcaatcttgaacattttacgctagatgggtatatgagcaaggatttccctaactggatttctcacatctccacataccttccttatctcacctttctcaccctttgcaatttaggagcatgtgataatcttccaccatttgggcaactgccaaatttaagatgtctagagatgaattatattcctaacatcacaaaaattggcaaggaattctatggagaaggtggaacctgtaggaaattaagaagtatacaattgaagtcaatggagaatttggtggaatggtggactgcactctcaggtgagaaaaatgaagagtcactgatccctaatttgcattgtttgtcaataaggaactgccccaagttgaagttcctaccatatcccccgaggagtatgttttggcacttggataatagcagcatggtttcgccaaaagagggatttggaaagctttcttcttacgcttttccttttctcatggtagtaagaaactgcaactttgctccagacaaatgggattggcttcaacacctccccacccttgagatattttatgttgagagcatcaatggcttgagggcattgccagaggccatgcaatgtttaaactctctcacaagactaaatttgtggtcattaaaggacctggagaaactgccagaatggttgggggacctcacttctctagaatacattcacatcaaagattgtcccatggtgacatctttgcctgtaagaatgaagtgtcttgctgctcttagagaactttgcctggcggagtgcagaggcctagaagtattgccagaatggctgggacatctgacttccctacaagagttctatatcatgagttgccccaacctgacttctttgcctgaaagcatgaagtgtcttactgctctcagagtacttcggctgatggagtgcgaaggcctagaagtattgccagaatggctgggacatctcacttccctacaagagttctatatcatccgttgccccaacctgacttctttgcctaaaagcatgaagtgtcttactgctctcagagtactttggctgacggagtgcgaaggcctagaagtattcccagaatggctaggacatctcatttccctacaagagttttacatcacctgttgctccaacctgacttgtttgcctgtaagcatgaagcgtcttactgctctgagattactctggttgggccagtgtgaaggtctagacgtattgccagaatggttgggacagctcatctcccttcaagaatttaacatcgtcggttgcccgaacttgacacattttcctgaaagcacacggaaccttaccgccttgaagaaactgaatatttggggatgtccgagtcttgttgagaggtgccaaggggaggatgctgacctgatttctcatatcccggaagtcttgttagaaggaaaaactagagaattgtggtcgacttccggagatgctgaagcatcgacttccggagcatcgactgctgaagaagctggaccatcgacttcttgagatccaatgagaggaagctagctggaagtgatcctgccatatacagagactaaagcaaattaagctgaaatgcaagaggtaataatgagaaacatgtaatgtttttcttcaacgtaactggacagtgatttcatcgtaattggttatgctggtgtattctgacaatctggaactctatgtgttaagctttgtcctgaacatatctggtctttcttttcagtttagagctagtgcagcactacctattgggctggaccttcatgccaatccaagctttattggtggcagagtcggaggcggcagcggcgctatgccccacggaagggatcctgcccggaagatcactcccgatgcattctgatcctaccaacagcggcgatggtggtgcatgcccatccttgtgagctccagttgagctgtaagagtctgcttctcccggatctagttcacctgatggtttcctctcatgcatgtgaataaaataccttgtgctagcaacacaaatattccacagagaatgcattgtcctcaaattgcttgcgttgctctggctaatttcgagctgtgtcctaaaatttatctcatctccttcgtttcacatcacactctcctcaaatttcgtcctggaaagagcgaatcaagaatgacgaacaccatgttggttgcacgactgaactgctaactgttctgcatatatatacttggcactgaccggtactagtacatctcgtgccttttcaggatagagacagaccatccagcaagcagtagttttttaacctatgtaccctggtccagtttttagcataagtaaacacttgcaacaacttttacttgcactattattttcggttattatttactccaaaatacaaaaaacatgtatcacaatatcgtaaaaactcaacaacacacttggcacctccacctgttaaaaaaacgcttggcacctccggatactttgtttggtacatcatgtgaattatattttttttttcgaaaagggggaaaacctcggcctctgcatcagaaagatgcatacggccatcttataaaccaaacaaagatagtgtcaacgtggttcgaaggtcttcaaaacagaaaaagtaaaacaagctcacacagagccatagagtggctagaaaccacaactagccaagataagataccacaacgatgattatactagatgcctatcctattacatgaccgccatccaaaccggttgaatatatcccgagctaccatctcccagcggacagatccagtaaccaaatgctccctggactccgtccgagtgagtggcgaccacgaacggatccgtgccgtagttcgaaatataacctgcaaaaagtggataatgatattctgttaaagaccaaatcatttctacaagtccagatagtccacagc